SEQUENCE LISTING (20 sequences)

SEQ ID NO: 1 - DNA, 689 bp, Homo sapiens
  source 1..689: mol_type = genomic DNA
ttcccatccacacatagagcttcagattctctttctttccccagagaccctcaaatatcctctcactcacagaatggtgtctctgcctgcctcgggttggccctgtgatttattttagttcttttcccttgttttttttttttcaaactctatacacttttgttttaaaaactgtggtttctcatgagccctattatctcattgatacctctcacctctgtggtgaggggaagaaatcatattttcagatgactcgtaaagggcaaagaaaaaaacccaaaatttcaaaatttccgtttaagtctcataatcaagaaaaggagaaacacagagagagagaaaaaaaaaactatgagaacccccccccaccccgtgattatcagcgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaaggtgagtgtccctgctctcccctaccagatgtgggccccattggaggagatggcagggaggtaggcacggcgggggggtcaggggccctctggtacagtgggatgtacccagctaccgtgattccagccaggtaaggtct

SEQ ID NO: 2 - DNA, 482 bp, Homo sapiens
  source 1..482: mol_type = genomic DNA
gcttcagattctctttctttccccagagaccctcaaatatcctctcactcacagaatggtgtctctgcctgcctcgggttggccctgtgatttattttagttcttttcccttgttttttttttttcaaactctatacacttttgttttaaaaactgtggtttctcatgagccctattatctcattgatacctctcacctctgtggtgaggggaagaaatcatattttcagatgactcgtaaagggcaaagaaaaaaacccaaaatttcaaaatttccgtttaagtctcataatcaagaaaaggagaaacacagagagagagaaaaaaaaaactatgagaacccccccccaccccgtgattatcagcgcacacactcatcgaaaaaaatttggattattagaagagagaggtctgcggcttccacaccgtacagcgtggtttttcttctcggtataaaagcaaagttgtttttgatacgtgac

SEQ ID NO: 3 - DNA, 420 bp, Homo sapiens
  source 1..420: mol_type = genomic DNA
gcttcagatcccttcttctgttcaacccagcgatcctccaacgtctcacaaacacaatgctgtctctacctgcctcgggatgcctttgtgatttgacttattttccctcagttttttttttctgactctacacacttttgtttaagaaattgtggtttctcatgagccctgttatctcattgataccttttacctctgtggtgaggggaagaaatcatattttcagatgacttgtaaagggcaaagaaaaaacccaaaatttcaaaatttccgtttaagtctcataagaaaagaataaacaaagtaagagagcaaagaaaaaaaaactacaagaaccccccccccaccctgcaattatcagcacacacactcatcaaaaaaaaattggattattagaagagcgaggtctgcggcttccac

SEQ ID NO: 4 - DNA, 424 bp, Homo sapiens
  source 1..424: mol_type = genomic DNA
gcttcagattctctttctttccccagagaccctcaaatatcctctcactcacagaatggtgtctctgcctgcctcgggttggccctgtgatttattttagttcttttcccttgttttttttttttcaaactctatacacttttgttttaaaaactgtggtttctcatgagccctattatctcattgatacctctcacctctgtggtgaggggaagaaatcatattttcagatgactcgtaaagggcaaagaaaaaaacccaaaatttcaaaatttccgtttaagtctcataatcaagaaaaggagaaacacagagagagagaaaaaaaaaactatgagaacccccccccaccccgtgattatcagcgcacacactcatcgaaaaaaatttggattattagaagagagaggtctgcggcttccac

SEQ ID NO: 5 - DNA, 150 bp, Homo sapiens
  source 1..150: mol_type = genomic DNA
tttaagtcttttgcacttgaaaatgagataactgttcaccccatgttggcttccagtctcctttatggcttcattttttccatttactgcagaggtcaaaagtgtgggtatgggagccagactgtctggaacaacctagcctcaactcaa

SEQ ID NO: 6 - DNA, 148 bp, Homo sapiens
  source 1..148: mol_type = genomic DNA
aagtcctttcctacttgaaaatgagataaatgttcacctatgttggcttctagtctcttttatggcttcattttttccatttactatagaggttaagagtgtgggtactggagccagactgtctgggacaaacccagcgtcaccccaa

SEQ ID NO: 7 - DNA, 341 bp, Homo sapiens
  source 1..341: mol_type = genomic DNA
tagattactcttttcttgtggggcttctgtgtatggttttgtgttttaagtcttttgcacttgaaaatgagataactgttcaccccatgttggcttccagtctcctttatggcttcattttttccatttactgcagaggtcaaaagtgtgggtatgggagccagactgtctggaacaacctagcctcaactcaagtcatctgtgtgaattttacccaggctcttaacctctctgtacctccatttcctcgtatgtactgtgatgattataacagtacctacctcagaggatctttctgaggattatttttattaatgatggtaggtgctcagcacaaggcc

SEQ ID NO: 8 - DNA, 343 bp, Homo sapiens
  source 1..343: mol_type = genomic DNA
taggttagtctttttttctgtggcttctgtctctggttttgtgcttagaaagtcctttcctacttgaaaatgagataaatgttcacctatgttggcttctagtctcttttatggcttcattttttccatttactatagaggttaagagtgtgggtactggagccagactgtctgggacaaacccagcgtcaccccaagccctatgtgtgatttttagccaggcacttaacctctccatacctccatttcctcatatgtactgcaatggttataatagtaccttcctcaggagtctttgtttagattaaaatttttaaccacagtaaatacttagcacaaggcc

SEQ ID NO: 9 - DNA, 403 bp, Homo sapiens
  source 1..403: mol_type = genomic DNA
cagatggacgtcacctaccacatccgctagcacccacatcaccctacctgggcctatccggctacaggatagactagccacttctcggaacgaaacctgtggggtagattatctgcccccttctcttcctccttgttgccgatgaagcccaatgcatccggccgccatgacgtcaatggcagaaaaatctggccaagttcaggttgtgacaacagggcccagatgtagaccccgataggaaaacatattctatgtcccagaaacaacctccatacagcttctaagaaacagtcaaacaggaacgccccaacagacagtgcaggaagctggctggccagcccagccctccaggtccctagtaccactagacagaccatatccaattcaggtcctctttctgaga

SEQ ID NO: 10 - DNA, 383 bp, Homo sapiens
  source 1..383: mol_type = genomic DNA
cagatggacatcacctaccacatccaccagcacccatgtcaccccacctgggccaagcctgctgcaggacagggcagccagttctcggaacgaaacctgtggggtggggtatctgccctcttctcttcctccgtggtgtcgatgaagcccggcgcatccggccgccatgacgtcaatggcggaaaaatctgggcaagtcgggggctgtgacaacagggcccagatgcagaccccgatatgaaaacataatctgtgtcccagaaacatcccccattcagcttctgagaaacccagtcagaaagggacgtcccaacagacagtgcaggaagccggctgcccagcccggccctctaggtcctctacccccagacagatcatctcca

SEQ ID NO: 11 - DNA, 426 bp, Homo sapiens
  source 1..426: mol_type = genomic DNA
tgggttttgcatggtagccagatggacgtcacctaccacatccgctagcacccacatcaccctacctgggcctatccggctacaggatagactagccacttctcggaacgaaacctgtggggtagattatctgcccccttctcttcctccttgttgccgatgaagcccaatgcatccggccgccatgacgtcaatggcagaaaaatctggccaagttcaggttgtgacaacagggcccagatgtagaccccgataggaaaacatattctatgtcccagaaacaacctccatacagcttctaagaaacagtcaaacaggaacgccccaacagacagtgcaggaagctggctggccagcccagccctccaggtccctagtaccactagacagaccatatccaattcaggtcctctttctgagaatgta

SEQ ID NO: 12 - DNA, 420 bp, Homo sapiens
  source 1..420: mol_type = genomic DNA
gggcttgtcatagtggccagatggacatcacctaccacatccaccagcacccatgtcaccccacctgggccaagcctgctgcaggacagggcagccagttctcggaacgaaacctgtggggtggggtatctgccctcttctcttcctccgtggtgtcgatgaagcccggcgcatccggccgccatgacgtcaatggcggaaaaatctgggcaagtcgggggctgtgacaacagggcccagatgcagaccccgatatgaaaacataatctgtgtcccagaaacatcccccattcagcttctgagaaacccagtcagaaagggacgtcccaacagacagtgcaggaagccggctgcccagcccggccctctaggtcctctacccccagacagatcatctccatgtccctgtctgagaatgta

SEQ ID NO: 13 - DNA, 207 bp, Homo sapiens
  source 1..207: mol_type = genomic DNA
cccggggcccagaatggggtaagcagggtggggtacttgggcctataggtgtcgacctttactgtggcatgtggcgggggggggggggggggctggggcacaggaagtggtttatgggtcccaggcaagtctgacttatgcagatattgcagggccaagaaaatccccactctccaggcttcagagattcaaggctttccccacccc

SEQ ID NO: 14 - DNA, 201 bp, Homo sapiens
  source 1..201: mol_type = genomic DNA
ccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctcccc

SEQ ID NO: 15 - DNA, 234 bp, Homo sapiens
  source 1..234: mol_type = genomic DNA
gtgaggcccggggcccagaatggggtaagcagggtggggtacttgggcctataggtgtcgacctttactgtggcatgtggcgggggggggggggggggctggggcacaggaagtggtttatgggtcccaggcaagtctgacttatgcagatattgcagggccaagaaaatccccactctccaggcttcagagattcaaggctttccccacccctcccaatcctcatcccgatag

SEQ ID NO: 16 - DNA, 220 bp, Homo sapiens
  source 1..220: mol_type = genomic DNA
gtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatcc

SEQ ID NO: 17 - DNA, 447 bp, Homo sapiens
  source 1..447: mol_type = genomic DNA
tcccctgaggtccaccaccatttccccagagggctggatcacggggggtagctattcttcaacagcacttcaaatcagcagcagcacacaggccttaaaacaataataagttgaaatgtatttgctaggaaagtcaccgacctacaaagaaaaccttatcgctgatctagcagcgcacaccagcctcccctttgcaagagctgagatcaaaagataaagaagctatcaaaaagccatctgcccacttaaaataacatctcaagtcacgttgggaaccacaaacatggggccagctaccaaaacaattgtctaaatgaactacttcaatttctccttaaaaccacccatgtattttaaaagaaaaacaccctctccacccaccttggcacggcaaggttttgatttgtctgttcccttcctttcacattcttgaaaatgaccaaactt

SEQ ID NO: 18 - DNA, 444 bp, Homo sapiens
  source 1..444: mol_type = genomic DNA
agtttggtcattttaaagagtgtgaaaggcagagaacagagaaatcaaaaccttgcagggccaaggtgggtggagagggtgtttttcttttaacatacatgggcggttttaaggagaaattgaagcagcctgttcagacaattgttttggtatctggccccaggtctgtggttcctaacatgacttgtgatattattttaagtgggcagatggctttttgatagcttctttatctttcgatctcagctcttgcaaaggggaggttggtgctcattgcaagatcagcgataagggtttctttgtaggtcggtggctttcttggtgagtacatttcaacatattattgttttagaacctgtgtgctgccagtgacttgcagcactgttgaagactagccaccctttgtgacctagccctcttgggaaatggcggaggatctcaggg

SEQ ID NO: 19 - DNA, 601 bp, Homo sapiens
  source 1..601: mol_type = genomic DNA
cagtgggcctgtggccaacgattctgaagccctctacagcaggcctcccacagataagaaaagggatcccctgaggtccaccaccatttccccagagggctggatcacggggggtagctattcttcaacagcacttcaaatcagcagcagcacacaggccttaaaacaataataagttgaaatgtatttgctaggaaagtcaccgacctacaaagaaaaccttatcgctgatctagcagcgcacaccagcctcccctttgcaagagctgagatcaaaagataaagaagctatcaaaaagccatctgcccacttaaaataacatctcaagtcacgttgggaaccacaaacatggggccagctaccaaaacaattgtctaaatgaactacttcaatttctccttaaaaccacccatgtattttaaaagaaaaacaccctctccacccaccttggcacggcaaggttttgatttgtctgttcccttcctttcacattcttgaaaatgaccaaacttcagtactcaactgtcttatcttccagaaagggctcccacaactgccgatggaataagaagtgattgaaatgcaggcgattctgggggc

SEQ ID NO: 20 - DNA, 531 bp, Homo sapiens
  source 1..531: mol_type = genomic DNA
cggctgtcatgggaaccctgtctgtaagatgcgacagtttgggtaaaggagtttggtcattttaaagagtgtgaaaggcagagaacagagaaatcaaaaccttgcagggccaaggtgggtggagagggtgtttttcttttaacatacatgggcggttttaaggagaaattgaagcagcctgttcagacaattgttttggtatctggccccaggtctgtggttcctaacatgacttgtgatattattttaagtgggcagatggctttttgatagcttctttatctttcgatctcagctcttgcaaaggggaggttggtgctcattgcaagatcagcgataagggtttctttgtaggtcggtggctttcttggtgagtacatttcaacatattattgttttagaacctgtgtgctgccagtgacttgcagcactgttgaagactagccaccctttgtgacctagccctcttgggaaatggcggaggatctcagggtatatcccttacctgtgggagccctatcagagggcttc

(19) United States
(12) Patent Application Publication
(10) Pub. No.: US 2025/0134927 A1
(43) Pub. Date: May 1, 2025

(54) COMPOSITIONS AND METHODS FOR TREATING OR PREVENTING AUTOIMMUNE DISEASES

(71) Applicant: Orchard Therapeutics (Europe) Limited, London (GB)

(72) Inventors: Jonathon Simeon MARKS-BLUTH, London (GB); Bobby GASPAR, London (GB); Pervinder SAGOO, St Albans (GB); Jia L. WOLFE, Winchester, MA (US); Chiara RECCHI, London (GB)

(21) Appl. No.: 18/690,622

(22) PCT Filed: Sep. 8, 2022
(86) PCT No.: PCT/US2022/076137

§ 371 Date: Mar. 8, 2024

Related U.S. Application Data
(60) Provisional application No. 63/241,836, filed on Sep. 8, 2021.

(51) Int. Cl. A61K 35/17; A61K 40/11; A61K 40/31; C07K 14/705; C07K 14/725; C07K 16/00; C12N 5/0783; C12N 9/22; C12N 15/113; C12N 15/86

(52) U.S. Cl. CPC A61K 35/17; A61K 40/11; A61K 40/31; C07K 14/7051; C07K 14/70521; C07K 16/00; C12N 5/0636; C12N 9/22; C12N 15/113; C12N 15/86; A61K 2239/13; A61K 2239/17; A61K 2239/21; C07K 2319/02; C07K 2319/03; C12N 2310/20; C12N 2510/00; C12N 2740/15043

(57) ABSTRACT

Described herein are compositions and methods for treating a subject having or at risk of developing an autoimmune disease. Using the compositions and methods of the disclosure, a patient may be provided pluripotent hematopoietic cells that are genetically modified to express an autoantigen-binding moiety (e.g., a chimeric antigen receptor) under the control of lineage-specific transcription regulatory elements that are active in CD4+CD25+ regulatory T (Treg) cells (e.g., a Foxp3 promoter).

### SEQUENCE LISTING

The instant application contains a Sequence Listing which has been submitted electronically in XML format and is hereby incorporated by reference in its entirety. Said XML copy, created on Aug. 24, 2022, is named 51139-032WO2_Sequence_Listing_8_24_22 and is 25,567 bytes in size.

### FIELD OF THE INVENTION

The disclosure relates to methods for treating autoimmune diseases by way of regulatory T cells derived from genetically modified pluripotent hematopoietic cells, as well as compositions that may be used in such methods.

### BACKGROUND OF THE INVENTION

Regulatory T (Treg) cells are a subset of T cells that play a critical role in suppressing the immune response, thereby maintaining homeostasis and self-tolerance. Treg deficiency or dysfunction is implicated in the pathology of several autoimmune diseases, and Treg cell therapy has been investigated as a potential therapeutic paradigm for these diseases. The development of Treg cell therapies has been hindered by difficulties associated with durability, stability, feasibility, manufacturing, and dosage of Treg cells. There remains a need for improved Treg cell therapies for the treatment of autoimmune diseases.

### SUMMARY OF THE INVENTION

The present disclosure relates to compositions and methods for the treatment of autoimmune diseases. In a first aspect, the disclosure provides a method of treating or preventing an autoimmune disease in a patient (e.g., a mammalian patient, such as a human patient) in need thereof by administering to the patient a population of pluripotent cells that include a nucleic acid cassette that encodes an autoantigen-binding protein. The nucleic acid cassette may be operably linked to one or more lineage-specific transcription regulatory elements that are active in CD4+CD25+ regulatory T (Treg) cells (i.e., preferentially active in cells of the Treg lineage as compared to other cell types (e.g., other hematopoietic cells)).

In a further aspect, the disclosure provides a method of suppressing activity and/or proliferation of a population of autoreactive effector immune cells in a patient (e.g., a mammalian patient, such as a human patient) diagnosed as having an autoimmune disease, the method including the step of administering to the patient a population of pluripotent cells that include a nucleic acid cassette that encodes an autoantigen-binding protein. The nucleic acid cassette may be operably linked to one or more lineage-specific transcription regulatory elements that are active in CD4+CD25+ Treg cells (i.e., specifically active in cells of the Treg lineage and not active in other cell types (e.g., other hematopoietic cells)).

In another aspect, the disclosure provides a method of inducing apoptosis of an autoreactive effector immune cell in a patient (e.g., a mammalian patient, such as a human patient) diagnosed as having an autoimmune disease, the method including the step of administering to the patient a population of pluripotent cells that include a nucleic acid cassette that encodes an autoantigen-binding protein. The nucleic acid cassette may be operably linked to one or more lineage-specific transcription regulatory elements that are active in CD4+CD25+ Treg cells (i.e., specifically active in cells of the Treg lineage and not active in other cell types (e.g., other hematopoietic cells)).

In another aspect, the disclosure provides a method of protecting endogenous tissue from an autoimmune response in a patient (e.g., a mammalian patient, such as a human patient) diagnosed as having an autoimmune disease, the method including the step of administering to the patient a population of pluripotent cells that include a nucleic acid cassette that encodes an autoantigen-binding protein. The nucleic acid cassette may be operably linked to one or more lineage-specific transcription regulatory elements that are active in CD4+CD25+ Treg cells (i.e., specifically active in cells of the Treg lineage and not active in other cell types (e.g., other hematopoietic cells)).

In another aspect, the disclosure provides a method of reducing inflammation in a patient (e.g., a mammalian patient, such as a human patient) diagnosed as having an autoimmune disease, the method including the step of administering to the patient a population of pluripotent cells that include a nucleic acid cassette that encodes an autoantigen-binding protein. The nucleic acid cassette may be operably linked to one or more lineage-specific transcription regulatory elements that are active in CD4+CD25+ Treg cells (i.e., specifically active in cells of the Treg lineage and not active in other cell types (e.g., other hematopoietic cells)).

In some embodiments of any of the above aspects, the pluripotent cells are pluripotent hematopoietic cells (e.g., hematopoietic stem cells (HSCs) or hematopoietic progenitor cells (HPCs)). In some embodiments, the pluripotent hematopoietic cells are embryonic stem cells. In some embodiments, the pluripotent hematopoietic cells are induced pluripotent stem cells. In some embodiments, the pluripotent hematopoietic cells are lymphoid progenitor cells. In some embodiments, the pluripotent hematopoietic cells are CD34+ cells (e.g., HSCs).

In some embodiments, the population of pluripotent hematopoietic cells is administered systemically to the patient. For example, the population of pluripotent hematopoietic cells may be administered to the patient by way of intravenous injection. In some embodiments, the population of pluripotent hematopoietic cells is administered locally to the patient.

In some embodiments, the pluripotent hematopoietic cells are autologous with respect to the patient. In some embodiments, the pluripotent hematopoietic cells are allogeneic with respect to the patient (e.g., HLA-matched allogeneic cells).

In some embodiments, the pluripotent hematopoietic cells (e.g., HSCs, HPCs, embryonic stem cells, induced pluripotent stem cells, lymphoid progenitor cells and/or CD34+ cells) are transduced ex vivo with a viral vector that includes the nucleic acid cassette that encodes the autoantigen-binding protein.

In some embodiments, the pluripotent hematopoietic cells are transduced with a viral vector selected from the group consisting of a Retroviridae family virus, an adenovirus, a parvovirus, a coronavirus, a rhabdovirus, a paramyxovirus, a picornavirus, an alphavirus, a herpes virus, and a poxvirus. In some embodiments, the viral vector is a Retroviridae family viral vector. In some embodiments, the Retroviridae family viral vector is a lentiviral vector. In some embodiments, the Retroviridae family viral vector is an alpharetroviral vector or a gammaretroviral vector. In some embodiments, the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

In some embodiments, the viral vector is a pseudotyped viral vector. In some embodiments, the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. In some embodiments, the pseudotyped viral vector is a pseudotyped lentiviral vector.

In some embodiments, the pseudotyped viral vector includes an envelope protein from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the pseudotyped viral vector includes a VSV-G envelope protein.

In some embodiments, the pluripotent hematopoietic cells (e.g., HSCs, HPCs, embryonic stem cells, induced pluripotent stem cells, lymphoid progenitor cells and/or CD34+ cells) are transfected ex vivo with a polynucleotide that includes the nucleic acid cassette that encodes the autoantigen-binding protein.

In some embodiments, the pluripotent hematopoietic cells are transfected using a cationic polymer, diethylaminoethyldextran, polyethylenimine, a cationic lipid, a liposome, calcium phosphate, an activated dendrimer, and/or a magnetic bead. In some embodiments, the pluripotent hematopoietic cells are transfected by way of electroporation, Nucleofection, squeeze-poration, sonoporation, optical transfection, Magnetofection, and/or impalefection.

In some embodiments, the nucleic acid cassette is part of a transposable element. In some embodiments, the nucleic acid cassette includes a transposase recognition and cleavage element for incorporation into a deoxyribonucleic acid (DNA) molecule of a pluripotent hematopoietic cell. In some embodiments, the DNA molecule is a nuclear or mitochondrial DNA molecule and the transposase recognition and cleavage element promotes incorporation into the nuclear or mitochondrial DNA molecule.

In some embodiments, the pluripotent hematopoietic cells are obtained by delivering to the cells a nuclease that catalyzes a single-strand break or a double-strand break at a target position within the genome of the cell. In some embodiments, the nuclease is delivered to the cells in combination with a guide RNA (gRNA) that hybridizes to the target position within the genome of the cell. In some embodiments, the nuclease is a clustered regularly interspaced short palindromic repeats (CRISPR)-associated protein. For example, in some embodiments, the CRISPR-associated protein is CRISPR-associated protein 9 (Cas9) or CRISPR-associated protein 12a (Cas12a). In some embodiments, the nuclease is a transcription activator-like effector nuclease, a meganuclease, or a zinc finger nuclease.

In some embodiments, while the cells are contacted with the nuclease, the cells are additionally contacted with a template polynucleotide that includes the nucleic acid cassette that encodes the autoantigen-binding protein. In some embodiments, the template polynucleotide includes a 5′ homology arm and a 3′ homology arm having nucleic acid sequences that are sufficiently similar to the nucleic acid sequences located 5′ to the target position and 3′ to the target position, respectively, to promote homologous recombination.

In some embodiments, the nuclease, gRNA, and/or template polynucleotide are delivered to the cells by contacting the cells with a viral vector that encodes the nuclease, gRNA, and/or template polynucleotide.

In some embodiments, the viral vector that encodes the nuclease, gRNA, and/or template polynucleotide is an AAV, an adenovirus, a parvovirus, a coronavirus, a rhabdovirus, a paramyxovirus, a picornavirus, an alphavirus, a herpes virus, a poxvirus, or a Retroviridae family virus.

In some embodiments, the viral vector that encodes the nuclease, gRNA, and/or template polynucleotide is a Retroviridae family virus. In some embodiments, the Retroviridae family virus is a lentiviral vector, alpharetroviral vector, or gammaretroviral vector. In some embodiments, the Retroviridae family virus that encodes the nuclease, gRNA, and/or template polynucleotide includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

In some embodiments, the viral vector that encodes the nuclease, gRNA, and/or template polynucleotide is an integration-deficient lentiviral vector. In some embodiments, the viral vector that encodes the nuclease, gRNA, and/or template polynucleotide is an AAV selected from the group consisting of AAV1, AAV2, AAV3, AAV4, AAV5, AAV6, AAV7, AAV8, AAV9, AAV10, and AAVrh74.

In some embodiments, the one or more lineage-specific transcription regulatory elements include a Foxp3 promoter.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 1. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 1. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 1. In some embodiments, the Foxp3 promoter has the nucleic acid sequence of SEQ ID NO: 1.

SEQ ID NO: 1:
TTCCCATCCACACATAGAGCTTCAGATTCTCTTTCTTTCCCCAGAGACC
CTCAAATATCCTCTCACTCACAGAATGGTGTCTCTGCCTGCCTCGGGTT
GGCCCTGTGATTTATTTTAGTTCTTTTCCCTTGTTTTTTTTTTTTCAAA
CTCTATACACTTTTGTTTTAAAAACTGTGGTTTCTCATGAGCCCTATTA
TCTCATTGATACCTCTCACCTCTGTGGTGAGGGGAAGAAATCATATTTT
CAGATGACTCGTAAAGGGCAAAGAAAAAAACCCAAAATTTCAAAATTTC
CGTTTAAGTCTCATAATCAAGAAAAGGAGAAACACAGAGAGAGAGAAAA
AAAAAACTATGAGAACCCCCCCCCACCCCGTGATTATCAGCGCACACAC
TCATCGAAAAAAATTTGGATTATTAGAAGAGAGAGGTCTGCGGCTTCCA
CACCGTACAGCGTGGTTTTTCTTCTCGGTATAAAAGCAAAGTTGTTTTT
GATACGTGACAGTTTCCCACAAGCCAGGCTGATCCTTTTCTGTCAGTCC
ACTTCACCAAGGTGAGTGTCCCTGCTCTCCCCTACCAGATGTGGGCCCC
ATTGGAGGAGATGGCAGGGAGGTAGGCACGGGGGGGGGTCAGGGGCCCT
CTGGTACAGTGGGATGTACCCAGCTACCGTGATTCCAGCCAGGTAAGGT
CT

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 2. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 2. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 2. In some embodiments, the Foxp3 promoter has the nucleic acid sequence of SEQ ID NO: 2.

SEQ ID NO: 2:
GCTTCAGATTCTCTTTCTTTCCCCAGAGACCCTCAAATATCCTCTCACT
CACAGAATGGTGTCTCTGCCTGCCTCGGGTTGGCCCTGTGATTTATTTT
AGTTCTTTTCCCTTGTTTTTTTTTTTTCAAACTCTATACACTTTTGTTT
TAAAAACTGTGGTTTCTCATGAGCCCTATTATCTCATTGATACCTCTCA
CCTCTGTGGTGAGGGGAAGAAATCATATTTTCAGATGACTCGTAAAGGG
CAAAGAAAAAAACCCAAAATTTCAAAATTTCCGTTTAAGTCTCATAATC
AAGAAAAGGAGAAACACAGAGAGAGAGAAAAAAAAAACTATGAGAACCC
CCCCCCACCCCGTGATTATCAGCGCACACACTCATCGAAAAAAATTTGG
ATTATTAGAAGAGAGAGGTCTGCGGCTTCCACACCGTACAGCGTGGTTT
TTCTTCTCGGTATAAAAGCAAAGTTGTTTTTGATACGTGAC

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 3. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 3. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 3. In some embodiments, the Foxp3 promoter has the nucleic acid sequence of SEQ ID NO: 3.

SEQ ID NO: 3
GCTTCAGATCCCTTCTTCTGTTCAACCCAGCGATCCTCCAACGTCTCAC
AAACACAATGCTGTCTCTACCTGCCTCGGGATGCCTTTGTGATTTGACT
TATTTTCCCTCAGTTTTTTTTTTCTGACTCTACACACTTTTGTTTAAGA
AATTGTGGTTTCTCATGAGCCCTGTTATCTCATTGATACCTTTTACCTC
TGTGGTGAGGGGAAGAAATCATATTTTCAGATGACTTGTAAAGGGCAAA
GAAAAAACCCAAAATTTCAAAATTTCCGTTTAAGTCTCATAAGAAAAGA
ATAAACAAAGTAAGAGAGCAAAGAAAAAAAAACTACAAGAACCCCCCCC
CCACCCTGCAATTATCAGCACACACACTCATCAAAAAAAAATTGGATTA
TTAGAAGAGCGAGGTCTGCGGCTTCCAC

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 4. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 4. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 4. In some embodiments, the Foxp3 promoter has the nucleic acid sequence of SEQ ID NO: 4.

SEQ ID NO: 4
GCTTCAGATTCTCTTTCTTTCCCCAGAGACCCTCAAATATCCTCTCACT
CACAGAATGGTGTCTCTGCCTGCCTCGGGTTGGCCCTGTGATTTATTTT
AGTTCTTTTCCCTTGTTTTTTTTTTTTCAAACTCTATACACTTTTGTTT
TAAAAACTGTGGTTTCTCATGAGCCCTATTATCTCATTGATACCTCTCA
CCTCTGTGGTGAGGGGAAGAAATCATATTTTCAGATGACTCGTAAAGGG
CAAAGAAAAAAACCCAAAATTTCAAAATTTCCGTTTAAGTCTCATAATC
AAGAAAAGGAGAAACACAGAGAGAGAGAAAAAAAAAACTATGAGAACCC
CCCCCCACCCCGTGATTATCAGCGCACACACTCATCGAAAAAAATTTGG
ATTATTAGAAGAGAGAGGTCTGCGGCTTCCAC

In some embodiments, the Foxp3 promoter specifically binds transcription factor Nr4a and/or Foxo.

In some embodiments, the one or more lineage-specific transcription regulatory elements include a CNS1 enhancer.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 5. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 5. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 5. In some embodiments, the CNS1 enhancer has the nucleic acid sequence of SEQ ID NO: 5.

SEQ ID NO: 5
TTTAAGTCTTTTGCACTTGAAAATGAGATAACTGTTCACCCCATGTTGG
CTTCCAGTCTCCTTTATGGCTTCATTTTTTCCATTTACTGCAGAGGTCA
AAAGTGTGGGTATGGGAGCCAGACTGTCTGGAACAACCTAGCCTCAACT
CAA

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 6. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 6. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 6. In some embodiments, the CNS1 enhancer has the nucleic acid sequence of SEQ ID NO: 6.

SEQ ID NO: 6
AAGTCCTTTCCTACTTGAAAATGAGATAAATGTTCACCTATGTTGGCTT
CTAGTCTCTTTTATGGCTTCATTTTTTCCATTTACTATAGAGGTTAAGA
GTGTGGGTACTGGAGCCAGACTGTCTGGGACAAACCCAGCGTCACCCCA
A

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 7. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 7. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 7. In some embodiments, the CNS1 enhancer has the nucleic acid sequence of SEQ ID NO: 7.

SEQ ID NO: 7
TAGATTACTCTTTTCTTGTGGGGCTTCTGTGTATGGTTTTGTGTTTTAA
GTCTTTTGCACTTGAAAATGAGATAACTGTTCACCCCATGTTGGCTTCC
AGTCTCCTTTATGGCTTCATTTTTTCCATTTACTGCAGAGGTCAAAAGT
GTGGGTATGGGAGCCAGACTGTCTGGAACAACCTAGCCTCAACTCAAGT
CATCTGTGTGAATTTTACCCAGGCTCTTAACCTCTCTGTACCTCCATTT
CCTCGTATGTACTGTGATGATTATAACAGTACCTACCTCAGAGGATCTT
TCTGAGGATTATTTTTATTAATGATGGTAGGTGCTCAGCACAAGGCC

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 8. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 8. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 8. In some embodiments, the CNS1 enhancer has the nucleic acid sequence of SEQ ID NO: 8.

SEQ ID NO: 8
TAGGTTAGTCTTTTTTTCTGTGGCTTCTGTCTCTGGTTTTGTGCTTAGA
AAGTCCTTTCCTACTTGAAAATGAGATAAATGTTCACCTATGTTGGCTT
CTAGTCTCTTTTATGGCTTCATTTTTTCCATTTACTATAGAGGTTAAGA
GTGTGGGTACTGGAGCCAGACTGTCTGGGACAAACCCAGCGTCACCCCA
AGCCCTATGTGTGATTTTTAGCCAGGCACTTAACCTCTCCATACCTCCA
TTTCCTCATATGTACTGCAATGGTTATAATAGTACCTTCCTCAGGAGTC
TTTGTTTAGATTAAAATTTTTAACCACAGTAAATACTTAGCACAAGGCC

In some embodiments, the CNS1 enhancer specifically binds transcription factor AP-1, NFAT, Smad3, and/or Foxo.

In some embodiments, the one or more lineage-specific transcription regulatory elements include a CNS2 enhancer.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 9. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 9. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 9. In some embodiments, the CNS2 enhancer has the nucleic acid sequence of SEQ ID NO: 9.

SEQ ID NO: 9
CAGATGGACGTCACCTACCACATCCGCTAGCACCCACATCACCCTACCT
GGGCCTATCCGGCTACAGGATAGACTAGCCACTTCTCGGAACGAAACCT
GTGGGGTAGATTATCTGCCCCCTTCTCTTCCTCCTTGTTGCCGATGAAG
CCCAATGCATCCGGCCGCCATGACGTCAATGGCAGAAAAATCTGGCCAA
GTTCAGGTTGTGACAACAGGGCCCAGATGTAGACCCCGATAGGAAAACA
TATTCTATGTCCCAGAAACAACCTCCATACAGCTTCTAAGAAACAGTCA
AACAGGAACGCCCCAACAGACAGTGCAGGAAGCTGGCTGGCCAGCCCAG
CCCTCCAGGTCCCTAGTACCACTAGACAGACCATATCCAATTCAGGTCC
TCTTTCTGAGA

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 10. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 10. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 10. In some embodiments, the CNS2 enhancer has the nucleic acid sequence of SEQ ID NO: 10.

SEQ ID NO: 10
CAGATGGACATCACCTACCACATCCACCAGCACCCATGTCACCCCACCT
GGGCCAAGCCTGCTGCAGGACAGGGCAGCCAGTTCTCGGAACGAAACCT
GTGGGGTGGGGTATCTGCCCTCTTCTCTTCCTCCGTGGTGTCGATGAAG
CCCGGCGCATCCGGCCGCCATGACGTCAATGGCGGAAAAATCTGGGCAA
GTCGGGGGCTGTGACAACAGGGCCCAGATGCAGACCCCGATATGAAAAC
ATAATCTGTGTCCCAGAAACATCCCCCATTCAGCTTCTGAGAAACCCAG
TCAGAAAGGGACGTCCCAACAGACAGTGCAGGAAGCCGGCTGCCCAGCC
CGGCCCTCTAGGTCCTCTACCCCCAGACAGATCATCTCCA

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 11. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 11. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 11. In some embodiments, the CNS2 enhancer has the nucleic acid sequence of SEQ ID NO: 11.

SEQ ID NO: 11
TGGGTTTTGCATGGTAGCCAGATGGACGTCACCTACCACATCCGCTAGC
ACCCACATCACCCTACCTGGGCCTATCCGGCTACAGGATAGACTAGCCA
CTTCTCGGAACGAAACCTGTGGGGTAGATTATCTGCCCCCTTCTCTTCC
TCCTTGTTGCCGATGAAGCCCAATGCATCCGGCCGCCATGACGTCAATG
GCAGAAAAATCTGGCCAAGTTCAGGTTGTGACAACAGGGCCCAGATGTA
GACCCCGATAGGAAAACATATTCTATGTCCCAGAAACAACCTCCATACA
GCTTCTAAGAAACAGTCAAACAGGAACGCCCCAACAGACAGTGCAGGAA
GCTGGCTGGCCAGCCCAGCCCTCCAGGTCCCTAGTACCACTAGACAGAC
CATATCCAATTCAGGTCCTCTTTCTGAGAATGTA

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 12. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 12. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 12. In some embodiments, the CNS2 enhancer has the nucleic acid sequence of SEQ ID NO: 12.

SEQ ID NO: 12
GGGCTTGTCATAGTGGCCAGATGGACATCACCTACCACATCCACCAGCA
CCCATGTCACCCCACCTGGGCCAAGCCTGCTGCAGGACAGGGCAGCCAG
TTCTCGGAACGAAACCTGTGGGGTGGGGTATCTGCCCTCTTCTCTTCCT
CCGTGGTGTCGATGAAGCCCGGCGCATCCGGCCGCCATGACGTCAATGG
CGGAAAAATCTGGGCAAGTCGGGGGCTGTGACAACAGGGCCCAGATGCA
GACCCCGATATGAAAACATAATCTGTGTCCCAGAAACATCCCCCATTCA
GCTTCTGAGAAACCCAGTCAGAAAGGGACGTCCCAACAGACAGTGCAGG
AAGCCGGCTGCCCAGCCCGGCCCTCTAGGTCCTCTACCCCCAGACAGAT
CATCTCCATGTCCCTGTCTGAGAATGTA

In some embodiments, the CNS2 enhancer specifically binds transcription factor Runx, Foxp3, Ets-1, CREB, Stat5, NFAT, and/or c-Rel.

In some embodiments, the one or more lineage-specific transcription regulatory elements include a CNS3 enhancer.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 13. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 13. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 13. In some embodiments, the CNS3 enhancer has the nucleic acid sequence of SEQ ID NO: 13.

SEQ ID NO: 13
CCCGGGGCCCAGAATGGGGTAAGCAGGGTGGGGTACTTGGGCCTATAGG
TGTCGACCTTTACTGTGGCATGTGGCGGGGGGGGGGGGGGGGGCTGGGG
CACAGGAAGTGGTTTATGGGTCCCAGGCAAGTCTGACTTATGCAGATAT
TGCAGGGCCAAGAAAATCCCCACTCTCCAGGCTTCAGAGATTCAAGGCT
TTCCCCACCCC

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 14. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 14. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 14. In some embodiments, the CNS3 enhancer has the nucleic acid sequence of SEQ ID NO: 14.

SEQ ID NO: 14
CCCTGGGCCCAGGATGGGGCAGGCAGGGTGGGGTACCTGGACCTACAGG
TGCCGACCTTTACTGTGGCACTGGGCGGGAGGGGGGCTGGCTGGGGCAC
AGGAAGTGGTTTCTGGGTCCCAGGCAAGTCTGTGACTTATGCAGATGTT
GCAGGGCCAAGAAAATCCCCACCTGCCAGGCCTCAGAGATTGGAGGCTC
TCCCC

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 15. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 15. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 15. In some embodiments, the CNS3 enhancer has the nucleic acid sequence of SEQ ID NO: 15.

SEQ ID NO: 15
GTGAGGCCCGGGGCCCAGAATGGGGTAAGCAGGGTGGGGTACTTGGGCC
TATAGGTGTCGACCTTTACTGTGGCATGTGGCGGGGGGGGGGGGGGGGG
CTGGGGCACAGGAAGTGGTTTATGGGTCCCAGGCAAGTCTGACTTATGC
AGATATTGCAGGGCCAAGAAAATCCCCACTCTCCAGGCTTCAGAGATTC
AAGGCTTTCCCCACCCCTCCCAATCCTCATCCCGATAG

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 16. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 16. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 16. In some embodiments, the CNS3 enhancer has the nucleic acid sequence of SEQ ID NO: 16.

SEQ ID NO: 16
GTGAGGCCCTGGGCCCAGGATGGGGCAGGCAGGGTGGGGTACCTGGACC
TACAGGTGCCGACCTTTACTGTGGCACTGGGCGGGAGGGGGGCTGGCTG
GGGCACAGGAAGTGGTTTCTGGGTCCCAGGCAAGTCTGTGACTTATGCA
GATGTTGCAGGGCCAAGAAAATCCCCACCTGCCAGGCCTCAGAGATTGG
AGGCTCTCCCCGACCTCCCAATCC

In some embodiments, the CNS3 enhancer specifically binds transcription factor Foxo and/or c-Rel.

In some embodiments, the one or more lineage-specific transcription regulatory elements include a CNS0 enhancer.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 17. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 17. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 17. In some embodiments, the CNS0 enhancer has the nucleic acid sequence of SEQ ID NO: 17.

SEQ ID NO: 17
TCCCCTGAGGTCCACCACCATTTCCCCAGAGGGCTGGATCACGGGGGGT
AGCTATTCTTCAACAGCACTTCAAATCAGCAGCAGCACACAGGCCTTAA
AACAATAATAAGTTGAAATGTATTTGCTAGGAAAGTCACCGACCTACAA
AGAAAACCTTATCGCTGATCTAGCAGCGCACACCAGCCTCCCCTTTGCA
AGAGCTGAGATCAAAAGATAAAGAAGCTATCAAAAAGCCATCTGCCCAC
TTAAAATAACATCTCAAGTCACGTTGGGAACCACAAACATGGGGCCAGC
TACCAAAACAATTGTCTAAATGAACTACTTCAATTTCTCCTTAAAACCA
CCCATGTATTTTAAAAGAAAAACACCCTCTCCACCCACCTTGGCACGGC
AAGGTTTTGATTTGTCTGTTCCCTTCCTTTCACATTCTTGAAAATGACC
AAACTT

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 18. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 18. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 18. In some embodiments, the CNS0 enhancer has the nucleic acid sequence of SEQ ID NO: 18.

SEQ ID NO: 18
AGTTTGGTCATTTTAAAGAGTGTGAAAGGCAGAGAACAGAGAAATCAAA
ACCTTGCAGGGCCAAGGTGGGTGGAGAGGGTGTTTTTCTTTTAACATAC
ATGGGGGGTTTTAAGGAGAAATTGAAGCAGCCTGTTCAGACAATTGTTT
TGGTATCTGGCCCCAGGTCTGTGGTTCCTAACATGACTTGTGATATTAT
TTTAAGTGGGCAGATGGCTTTTTGATAGCTTCTTTATCTTTCGATCTCA
GCTCTTGCAAAGGGGAGGTTGGTGCTCATTGCAAGATCAGCGATAAGGG
TTTCTTTGTAGGTCGGTGGCTTTCTTGGTGAGTACATTTCAACATATTA
TTGTTTTAGAACCTGTGTGCTGCCAGTGACTTGCAGCACTGTTGAAGAC
TAGCCACCCTTTGTGACCTAGCCCTCTTGGGAAATGGCGGAGGATCTCA
GGG

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 19. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 19. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 19. In some embodiments, the CNS0 enhancer has the nucleic acid sequence of SEQ ID NO: 19.

SEQ ID NO: 19
CAGTGGGCCTGTGGCCAACGATTCTGAAGCCCTCTACAGCAGGCCTCCC
ACAGATAAGAAAAGGGATCCCCTGAGGTCCACCACCATTTCCCCAGAGG
GCTGGATCACGGGGGGTAGCTATTCTTCAACAGCACTTCAAATCAGCAG
CAGCACACAGGCCTTAAAACAATAATAAGTTGAAATGTATTTGCTAGGA
AAGTCACCGACCTACAAAGAAAACCTTATCGCTGATCTAGCAGCGCACA
CCAGCCTCCCCTTTGCAAGAGCTGAGATCAAAAGATAAAGAAGCTATCA
AAAAGCCATCTGCCCACTTAAAATAACATCTCAAGTCACGTTGGGAACC
ACAAACATGGGGCCAGCTACCAAAACAATTGTCTAAATGAACTACTTCA
ATTTCTCCTTAAAACCACCCATGTATTTTAAAAGAAAAACACCCTCTCC
ACCCACCTTGGCACGGCAAGGTTTTGATTTGTCTGTTCCCTTCCTTTCA
CATTCTTGAAAATGACCAAACTTCAGTACTCAACTGTCTTATCTTCCAG
AAAGGGCTCCCACAACTGCCGATGGAATAAGAAGTGATTGAAATGCAGG
CGATTCTGGGGGC

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 20. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 20. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 20. In some embodiments, the CNS0 enhancer has the nucleic acid sequence of SEQ ID NO: 20.

SEQ ID NO: 20
CGGCTGTCATGGGAACCCTGTCTGTAAGATGCGACAGTTTGGGTAAAGG
AGTTTGGTCATTTTAAAGAGTGTGAAAGGCAGAGAACAGAGAAATCAAA
ACCTTGCAGGGCCAAGGTGGGTGGAGAGGGTGTTTTTCTTTTAACATAC
ATGGGCGGTTTTAAGGAGAAATTGAAGCAGCCTGTTCAGACAATTGTTT
TGGTATCTGGCCCCAGGTCTGTGGTTCCTAACATGACTTGTGATATTAT
TTTAAGTGGGCAGATGGCTTTTTGATAGCTTCTTTATCTTTCGATCTCA
GCTCTTGCAAAGGGGAGGTTGGTGCTCATTGCAAGATCAGCGATAAGGG
TTTCTTTGTAGGTCGGTGGCTTTCTTGGTGAGTACATTTCAACATATTA
TTGTTTTAGAACCTGTGTGCTGCCAGTGACTTGCAGCACTGTTGAAGAC
TAGCCACCCTTTGTGACCTAGCCCTCTTGGGAAATGGCGGAGGATCTCA
GGGTATATCCCTTACCTGTGGGAGCCCTATCAGAGGGCTTC

In some embodiments, the CNS0 enhancer specifically binds transcription factor Satb1 and/or Stat5.

In some embodiments, the nucleic acid cassette is operably linked to a riboswitch. In some embodiments, binding of a ligand to the riboswitch induces expression of the nucleic acid cassette. In some embodiments, binding of a ligand to the riboswitch represses expression of the nucleic acid cassette.

In some embodiments, the autoantigen-binding protein is a single-chain polypeptide. In some embodiments, the autoantigen-binding protein is a chimeric antigen receptor (CAR).

In some embodiments, the chimeric antigen receptor includes an antigen recognition domain, a hinge domain, a transmembrane domain, and one or more intracellular signaling domains.

In some embodiments, the one or more intracellular signaling domains include one or more primary intracellular signaling domains and optionally one or more costimulatory intracellular signaling domains.

In some embodiments, the antigen recognition domain is a single-chain antibody fragment (e.g., a single-chain Fv molecule (scFv)).

In some embodiments, the hinge domain is a CD28, CD8, IgG1/IgG4, CD4, CD7, or IgD hinge domain.

In some embodiments, the hinge domain is a CD28 hinge domain.

In some embodiments, the transmembrane domain includes a CD28, CD3 zeta, CD8, FcRIγ, CD4, CD7, OX40, or MHC (H2-Kb) transmembrane domain.

In some embodiments, the transmembrane domain includes a CD28 transmembrane domain.

In some embodiments, the one or more primary intracellular signaling domains are selected from the group consisting of a CD3 zeta, FcR gamma, FcR beta, CD3 gamma, CD3 delta, CD3 epsilon, CD5, CD22, CD79a, CD79b, CD278 (ICOS), CD66d, DAP10, and a DAP12 intracellular signaling domain.

In some embodiments, at least one of the one or more primary intracellular signaling domains is a CD3 zeta intracellular signaling domain.

In some embodiments, the one or more costimulatory intracellular signaling domains are selected from the group consisting of a CD27, CD28, 4-1 BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, CD83, CDS, ICAM-1, LFA-1 (CD11a/CD18), an MHC class I molecule, BTLA, and a Toll ligand receptor intracellular signaling domain.

In some embodiments, at least one of the one or more co-stimulatory intracellular signaling domains is a CD28 intracellular signaling domain.

In some embodiments, the chimeric antigen receptor includes an N-terminal leader sequence. In some embodiments, the antigen recognition domain includes an N-terminal leader sequence. In some embodiments, the N-terminal leader sequence of the antigen recognition domain is cleaved from the antigen recognition domain during cellular processing and localization of the chimeric antigen receptor to the cellular membrane.

In some embodiments, the autoantigen-binding protein is a multi-chain protein. In some embodiments, the autoantigen-binding protein is a full-length antibody, a dual-variable immunoglobulin domain, a diabody, a triabody, a nanobody, an antibody-like protein scaffold, a Fab fragment, or a F(ab′)₂ molecule.

In some embodiments, the autoimmune disease is type 1 diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barré, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Ménière's Disease, Mixed Connective Tissue Disease, Multiple Sclerosis, Myasthenia Gravis, Neuromyelitis Optica, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, or Wegener's Granulomatosis.

In some embodiments, the autoantigen is myelin oligodendrocyte glycoprotein, aquaporin 4, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double-stranded DNA, single-stranded DNA, transferrin, fetuin, factor II, factor VII, fibrin, fibrinogen, C1, C1q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

In some embodiments, the autoimmune disease is multiple sclerosis and the autoantigen is myelin oligodendrocyte glycoprotein.

In some embodiments, the autoimmune disease is type 1 diabetes and the autoantigen is insulin, GAD-65, IA-2, or ZnT8.

In some embodiments, the autoimmune disease is rheumatoid arthritis and the autoantigen is collagen II, the Fc portion of immunoglobin, citrullinated peptides, carbamylated peptides, or HSP65.

In some embodiments, the autoimmune disease is myasthenia gravis and the autoantigen is AChR, MuSK, or LRP4.

In some embodiments, the autoimmune disease is lupus and the autoantigen is histone II A.

In some embodiments, the autoimmune disease is hypothyroidism and the autoantigen is a protein expressed in the thyroid gland.

In some embodiments, the autoimmune disease is Graves' disease and the autoantigen is the thyrotrophin receptor.

In some embodiments, the autoimmune disease is pemphigus vulgaris and the autoantigen is double-stranded DNA.

In some embodiments, the autoimmune disease is psoriasis and the autoantigen is double-stranded DNA.

In some embodiments, the autoimmune disease is neuromyelitis optica and the autoantigen is aquaporin 4.

In some embodiments, prior to administering the population of pluripotent hematopoietic cells to the patient, a population of precursor cells is isolated from the patient or a donor, and the precursor cells are expanded and genetically modified ex vivo to yield the population of cells being administered to the patient. In some embodiments, the precursor cells are CD34+ HSCs, and the precursor cells are expanded without substantial loss of HSC functional potential. In some embodiments, prior to isolation of the precursor cells from the patient or donor, the patient or donor is administered one or more pluripotent hematopoietic cell mobilization agents.

In some embodiments, prior to administering the population of pluripotent hematopoietic cells to the patient, a population of endogenous pluripotent hematopoietic cells is ablated in the patient by administration of one or more conditioning agents to the patient.

In some embodiments, the method includes ablating a population of endogenous pluripotent hematopoietic cells in the patient by administering to the patient one or more conditioning agents prior to administering the population of pluripotent hematopoietic cells to the patient.

In some embodiments, the one or more conditioning agents are non-myeloablative conditioning agents. In some embodiments, the one or more conditioning agents deplete a population of CD34+ cells in the patient. In some embodiments, the depleted CD34+ cells are lymphoid progenitor cells. In some embodiments, the one or more conditioning agents include an antibody or antigen-binding fragment thereof. In some embodiments, the antibody or antigen-binding fragment thereof binds to CD117, HLA-DR, CD34, CD90, CD45, or CD133. In some embodiments, the antibody or antigen-binding fragment thereof binds to CD117. In some embodiments, the antibody or antigen-binding fragment thereof is conjugated to a cytotoxin.

In some embodiments, upon administration of the population of pluripotent hematopoietic cells to the patient, the administered cells, or progeny thereof, differentiate into CD4+CD25+ Treg cells.

In some embodiments, the patient is a mammal and the cells are mammalian cells. In some embodiments, the mammal is a human and the cells are human cells.

In another aspect, the disclosure provides a pharmaceutical composition that includes (i) a population of pluripotent cells (e.g., pluripotent hematopoietic cells) that include a nucleic acid cassette that encodes an autoantigen-binding protein. The nucleic acid cassette may be operably linked to one or more lineage-specific transcription regulatory elements that are active in CD4+CD25+ Treg cells (i.e., specifically active in cells of the Treg lineage and not active in other cell types (e.g., other hematopoietic cells)), and (ii) one or more pharmaceutically acceptable excipients, carriers, or diluents.

In some embodiments of any of the above aspects, the pluripotent cells are pluripotent hematopoietic cells (e.g., HSCs or HPCs). In some embodiments, the pluripotent hematopoietic cells are embryonic stem cells. In some embodiments, the pluripotent hematopoietic cells are induced pluripotent stem cells. In some embodiments, the pluripotent hematopoietic cells are lymphoid progenitor cells. In some embodiments, the pluripotent hematopoietic cells are CD34+ cells (e.g., HSCs).

In some embodiments, the pluripotent hematopoietic cells are transduced ex vivo with a viral vector that includes the nucleic acid cassette that encodes the autoantigen-binding protein.

In some embodiments, the viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus. In some embodiments, the viral vector is a Retroviridae family viral vector. In some embodiments, the Retroviridae family viral vector is a lentiviral vector. In some embodiments, the Retroviridae family viral vector is an alpharetroviral vector or a gammaretroviral vector.

In some embodiments, the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

In some embodiments, the viral vector is a pseudotyped viral vector. In some embodiments, the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. In some embodiments, the pseudotyped viral vector is a pseudotyped lentiviral vector.

In some embodiments, the pseudotyped viral vector includes an envelope protein from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the pseudotyped viral vector includes a VSV-G envelope protein.

In some embodiments, the pluripotent hematopoietic cells are transfected ex vivo with a polynucleotide that includes the nucleic acid cassette that encodes the autoantigen-binding protein.

In some embodiments, the pluripotent hematopoietic cells are transfected using a cationic polymer, diethylaminoethyldextran, polyethylenimine, a cationic lipid, a liposome, calcium phosphate, an activated dendrimer, and/or a magnetic bead. In some embodiments, the pluripotent hematopoietic cells are transfected by way of electroporation, Nucleofection, squeeze-poration, sonoporation, optical transfection, Magnetofection, and/or impalefection.

In some embodiments, the nucleic acid cassette is part of a transposable element. In some embodiments, the nucleic acid cassette includes a transposase recognition and cleavage element for incorporation into a deoxyribonucleic acid (DNA) molecule of a pluripotent hematopoietic cell. In some embodiments, the DNA molecule is a nuclear or mitochondrial DNA molecule and the transposase recognition and cleavage element promotes incorporation into the nuclear or mitochondrial DNA molecule.

In some embodiments, the pluripotent hematopoietic cells are obtained by delivering to the cells a nuclease that catalyzes a single-strand break or a double-strand break at a target position within the genome of the cell. In some embodiments, the nuclease is delivered to the cells in combination with a guide RNA (gRNA) that hybridizes to the target position within the genome of the cell. In some embodiments, the nuclease is a clustered regularly interspaced short palindromic repeats (CRISPR)-associated protein. In some embodiments, the CRISPR-associated protein is CRISPR-associated protein 9 (Cas9) or CRISPR-associated protein 12a (Cas12a). In some embodiments, the nuclease is a transcription activator-like effector nuclease, a meganuclease, or a zinc finger nuclease.

In some embodiments, while the cells are contacted with the nuclease, the cells are additionally contacted with a template polynucleotide that includes the nucleic acid cassette that encodes the autoantigen-binding protein. In some embodiments, the template polynucleotide includes a 5′ homology arm and a 3′ homology arm having nucleic acid sequences that are sufficiently similar to the nucleic acid sequences located 5′ to the target position and 3′ to the target position, respectively, to promote homologous recombination.

In some embodiments, the nuclease, gRNA, and/or template polynucleotide are delivered to the cells by contacting the cells with a viral vector that encodes the nuclease, gRNA, and/or template polynucleotide.

In some embodiments, the viral vector that encodes the nuclease, gRNA, and/or template polynucleotide is an AAV, an adenovirus, a parvovirus, a coronavirus, a rhabdovirus, a paramyxovirus, a picornavirus, an alphavirus, a herpes virus, a poxvirus, or a Retroviridae family virus.

In some embodiments, the viral vector that encodes the nuclease, gRNA, and/or template polynucleotide is a Retroviridae family virus. In some embodiments, the Retroviridae family virus is a lentiviral vector, alpharetroviral vector, or gammaretroviral vector. In some embodiments, the Retroviridae family virus that encodes the nuclease, gRNA, and/or template polynucleotide includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3′-self inactivating LTR.

In some embodiments, the viral vector that encodes the nuclease, gRNA, and/or template polynucleotide is an integration-deficient lentiviral vector. In some embodiments, the viral vector that encodes the nuclease, gRNA, and/or template polynucleotide is an AAV selected from the group consisting of AAV1, AAV2, AAV3, AAV4, AAV5, AAV6, AAV7, AAV8, AAV9, AAV10, and AAVrh74.

In some embodiments, the one or more lineage-specific transcription regulatory elements include a Foxp3 promoter.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 1. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 1. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 1. In some embodiments, the Foxp3 promoter has the nucleic acid sequence of SEQ ID NO: 1.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 2. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 2. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 2. In some embodiments, the Foxp3 promoter has the nucleic acid sequence of SEQ ID NO: 2.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 3. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 3. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 3. In some embodiments, the Foxp3 promoter has the nucleic acid sequence of SEQ ID NO: 3.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 4. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 4. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 4. In some embodiments, the Foxp3 promoter has the nucleic acid sequence of SEQ ID NO: 4.

In some embodiments, the Foxp3 promoter specifically binds transcription factor Nr4a and/or Foxo.

In some embodiments, the one or more lineage-specific transcription regulatory elements include a CNS1 enhancer.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 5. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 5. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 5. In some embodiments, the CNS1 enhancer has the nucleic acid sequence of SEQ ID NO: 5.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 6. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 6. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 6. In some embodiments, the CNS1 enhancer has the nucleic acid sequence of SEQ ID NO: 6.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 7. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 7. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 7. In some embodiments, the CNS1 enhancer has the nucleic acid sequence of SEQ ID NO: 7.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 8. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 8. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 8. In some embodiments, the CNS1 enhancer has the nucleic acid sequence of SEQ ID NO: 8.

In some embodiments, the CNS1 enhancer specifically binds transcription factor AP-1, NFAT, Smad3, and/or Foxo.

In some embodiments, the one or more lineage-specific transcription regulatory elements include a CNS2 enhancer.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 9. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 9. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 9. In some embodiments, the CNS2 enhancer has the nucleic acid sequence of SEQ ID NO: 9.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 10. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 10. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 10. In some embodiments, the CNS2 enhancer has the nucleic acid sequence of SEQ ID NO: 10.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 11. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 11. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 11. In some embodiments, the CNS2 enhancer has the nucleic acid sequence of SEQ ID NO: 11.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 12. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 12. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 12. In some embodiments, the CNS2 enhancer has the nucleic acid sequence of SEQ ID NO: 12.

In some embodiments, the CNS2 enhancer specifically binds transcription factor Runx, Foxp3, Ets-1, CREB, Stat5, NFAT, and/or c-Rel.

In some embodiments, the one or more lineage-specific transcription regulatory elements include a CNS3 enhancer.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 13. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 13. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 13. In some embodiments, the CNS3 enhancer has the nucleic acid sequence of SEQ ID NO: 13.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 14. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 14. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 14. In some embodiments, the CNS3 enhancer has the nucleic acid sequence of SEQ ID NO: 14.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 15. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 15. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 15. In some embodiments, the CNS3 enhancer has the nucleic acid sequence of SEQ ID NO: 15.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 16. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 16. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 16. In some embodiments, the CNS3 enhancer has the nucleic acid sequence of SEQ ID NO: 16.

In some embodiments, the CNS3 enhancer specifically binds transcription factor Foxo and/or c-Rel.

In some embodiments, the one or more lineage-specific transcription regulatory elements include a CNS0 enhancer.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 17. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 17. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 17. In some embodiments, the CNS0 enhancer has the nucleic acid sequence of SEQ ID NO: 17.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 18. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 18. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 18. In some embodiments, the CNS0 enhancer has the nucleic acid sequence of SEQ ID NO: 18.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 19. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 19. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 19. In some embodiments, the CNS0 enhancer has the nucleic acid sequence of SEQ ID NO: 19.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 20. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 20. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 20. In some embodiments, the CNS0 enhancer has the nucleic acid sequence of SEQ ID NO: 20.

In some embodiments, the CNS0 enhancer specifically binds transcription factor Satb1 and/or Stat5.

In some embodiments, the nucleic acid cassette is operably linked to a riboswitch. In some embodiments, binding of a ligand to the riboswitch induces expression of the nucleic acid cassette.

In some embodiments, the autoantigen-binding protein is a single-chain polypeptide. In some embodiments, the autoantigen-binding protein is a chimeric antigen receptor.

In some embodiments, the chimeric antigen receptor includes an antigen recognition domain, a hinge domain, a transmembrane domain, and one or more intracellular signaling domains.

In some embodiments, the one or more intracellular signaling domains include one or more primary intracellular signaling domains and optionally one or more costimulatory intracellular signaling domains.

In some embodiments, the antigen recognition domain is a single-chain antibody fragment (e.g., a single-chain Fv molecule (scFv)).

In some embodiments, the hinge domain is a CD28, CD8, IgG1/IgG4, CD4, CD7, or IgD hinge domain.

In some embodiments, the hinge domain is a CD28 hinge domain.

In some embodiments, the transmembrane domain includes a CD28, CD3 zeta, CD8, FcRIγ, CD4, CD7, OX40, or MHC (H2-Kb) transmembrane domain.

In some embodiments, the transmembrane domain includes a CD28 transmembrane domain.

In some embodiments, the one or more primary intracellular signaling domains are selected from the group consisting of a CD3 zeta, FcR gamma, FcR beta, CD3 gamma, CD3 delta, CD3 epsilon, CD5, CD22, CD79a, CD79b, CD278 (ICOS), CD66d, DAP10, and a DAP12 intracellular signaling domain.

In some embodiments, at least one of the one or more primary intracellular signaling domains is a CD3 zeta intracellular signaling domain.

In some embodiments, the one or more costimulatory intracellular signaling domains are selected from the group consisting of a CD27, CD28, 4-1 BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, CD83, CDS, ICAM-1, LFA-1 (CD11a/CD18), an MHC class I molecule, BTLA, and a Toll ligand receptor intracellular signaling domain.

In some embodiments, at least one of the one or more co-stimulatory intracellular signaling domains is a CD28 intracellular signaling domain.

In some embodiments, the chimeric antigen receptor includes an N-terminal leader sequence. In some embodiments, the antigen recognition domain includes an N-terminal leader sequence. In some embodiments, the N-terminal leader sequence of the antigen recognition domain is cleaved from the antigen recognition domain during cellular processing and localization of the chimeric antigen receptor to the cellular membrane.

In some embodiments, the autoantigen-binding protein is a multi-chain protein. In some embodiments, the autoantigen-binding protein is a full-length antibody, a dual-variable immunoglobulin domain, a diabody, a triabody, an antibody-like protein scaffold, a Fab fragment, or a F(ab′)₂ molecule.

In some embodiments, the autoantigen is myelin oligodendrocyte glycoprotein, aquaporin 4, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double stranded DNA, single stranded DNA, transferrin, fetuin, factor II, factor VII, fibrin, fibrinogen, C1, C1q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

In another aspect, the disclosure provides a kit including a pharmaceutical composition as described herein. The kit may further include a package insert instructing a user of the kit to administer the pharmaceutical composition to a human patient having an autoimmune disease. The package insert may instruct a user of the kit to perform a method as described herein.

In another aspect, the disclosure provides a nucleic acid cassette encoding an autoantigen-binding protein. The nucleic acid cassette may be operably linked to one or more lineage-specific transcription regulatory elements that are active in CD4+CD25+ Treg cells (i.e., specifically active in cells of the Treg lineage and not active in other cell types (e.g., other hematopoietic cells)).

In some embodiments, the one or more lineage-specific transcription regulatory elements include a Foxp3 promoter.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 1. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 1. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 1. In some embodiments, the Foxp3 promoter has the nucleic acid sequence of SEQ ID NO: 1.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 2. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 2. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 2. In some embodiments, the Foxp3 promoter has the nucleic acid sequence of SEQ ID NO: 2.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 3. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 3. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 3. In some embodiments, the Foxp3 promoter has the nucleic acid sequence of SEQ ID NO: 3.

In some embodiments, the Foxp3 promoter has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 4. In some embodiments, the Foxp3 promoter has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 4. In some embodiments, the Foxp3 promoter has a nucleic acid sequence that is at least 95% identical (e.g., 95%, 96%, 97%, 98%, 99%, or 100% identical) to the nucleic acid sequence of SEQ ID NO: 4. In some embodiments, the Foxp3 promoter has the nucleic acid sequence of SEQ ID NO: 4.

In some embodiments, the Foxp3 promoter specifically binds transcription factor Nr4a and/or Foxo.

In some embodiments, the one or more lineage-specific transcription regulatory elements include a CNS1 enhancer.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 5. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 5. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 5. In some embodiments, the CNS1 enhancer has the nucleic acid sequence of SEQ ID NO: 5.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 6. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 6. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 6. In some embodiments, the CNS1 enhancer has the nucleic acid sequence of SEQ ID NO: 6.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 7. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 7. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 7. In some embodiments, the CNS1 enhancer has the nucleic acid sequence of SEQ ID NO: 7.

In some embodiments, the CNS1 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 8. In some embodiments, the CNS1 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 8. In some embodiments, the CNS1 enhancer has a nucleic acid sequence that is at least 95% identical (e.g., 95%, 96%, 97%, 98%, 99%, or 100% identical) to the nucleic acid sequence of SEQ ID NO: 8. In some embodiments, the CNS1 enhancer has the nucleic acid sequence of SEQ ID NO: 8.

In some embodiments, the CNS1 enhancer specifically binds transcription factor AP-1, NFAT, Smad3, and/or Foxo.

In some embodiments, the one or more lineage-specific transcription regulatory elements include a CNS2 enhancer.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 9. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 9. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 9. In some embodiments, the CNS2 enhancer has the nucleic acid sequence of SEQ ID NO: 9.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 10. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 10. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 10. In some embodiments, the CNS2 enhancer has the nucleic acid sequence of SEQ ID NO: 10.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 11. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 11. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 11. In some embodiments, the CNS2 enhancer has the nucleic acid sequence of SEQ ID NO: 11.

In some embodiments, the CNS2 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 12. In some embodiments, the CNS2 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 12. In some embodiments, the CNS2 enhancer has a nucleic acid sequence that is at least 95% identical (e.g., 95%, 96%, 97%, 98%, 99%, or 100% identical) to the nucleic acid sequence of SEQ ID NO: 12. In some embodiments, the CNS2 enhancer has the nucleic acid sequence of SEQ ID NO: 12.

In some embodiments, the CNS2 enhancer specifically binds transcription factor Runx, Foxp3, Ets-1, CREB, Stat5, NFAT, and/or c-Rel.

In some embodiments, the one or more lineage-specific transcription regulatory elements include a CNS3 enhancer.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 13. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 13. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 13. In some embodiments, the CNS3 enhancer has the nucleic acid sequence of SEQ ID NO: 13.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 14. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 14. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 14. In some embodiments, the CNS3 enhancer has the nucleic acid sequence of SEQ ID NO: 14.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 15. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 15. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 15. In some embodiments, the CNS3 enhancer has the nucleic acid sequence of SEQ ID NO: 15.

In some embodiments, the CNS3 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 16. In some embodiments, the CNS3 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 16. In some embodiments, the CNS3 enhancer has a nucleic acid sequence that is at least 95% identical (e.g., 95%, 96%, 97%, 98%, 99%, or 100% identical) to the nucleic acid sequence of SEQ ID NO: 16. In some embodiments, the CNS3 enhancer has the nucleic acid sequence of SEQ ID NO: 16.

In some embodiments, the CNS3 enhancer specifically binds transcription factor Foxo and/or c-Rel.

In some embodiments, the one or more lineage-specific transcription regulatory elements include a CNS0 enhancer.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 17. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 17. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 17. In some embodiments, the CNS0 enhancer has the nucleic acid sequence of SEQ ID NO: 17.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 18. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 18. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 18. In some embodiments, the CNS0 enhancer has the nucleic acid sequence of SEQ ID NO: 18.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 19. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 19. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 19. In some embodiments, the CNS0 enhancer has the nucleic acid sequence of SEQ ID NO: 19.

In some embodiments, the CNS0 enhancer has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 20. In some embodiments, the CNS0 enhancer has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 20. In some embodiments, the CNS0 enhancer has a nucleic acid sequence that is at least 95% identical (e.g., 95%, 96%, 97%, 98%, 99%, or 100% identical) to the nucleic acid sequence of SEQ ID NO: 20. In some embodiments, the CNS0 enhancer has the nucleic acid sequence of SEQ ID NO: 20.

In some embodiments, the CNS0 enhancer specifically binds transcription factor Satb1 and/or Stat5.

In some embodiments, the nucleic acid cassette is operably linked to a riboswitch. In some embodiments, binding of a ligand to the riboswitch induces expression of the nucleic acid cassette.

In some embodiments, the autoantigen-binding protein is a single-chain polypeptide. In some embodiments, the autoantigen-binding protein is a chimeric antigen receptor.

In some embodiments, the chimeric antigen receptor includes an antigen recognition domain, a hinge domain, a transmembrane domain, and one or more intracellular signaling domains.

In some embodiments, the one or more intracellular signaling domains include one or more primary intracellular signaling domains and optionally one or more costimulatory intracellular signaling domains.

In some embodiments, the antigen recognition domain is a single-chain antibody fragment (e.g., a single-chain Fv molecule (scFv)).

In some embodiments, the hinge domain is a CD28, CD8, IgG1/IgG4, CD4, CD7, or IgD hinge domain.

In some embodiments, the hinge domain is a CD28 hinge domain.

In some embodiments, the transmembrane domain includes a CD28, CD3 zeta, CD8, FcRIγ, CD4, CD7, OX40, or MHC (H2-Kb) transmembrane domain.

In some embodiments, the transmembrane domain includes a CD28 transmembrane domain.

In some embodiments, the one or more primary intracellular signaling domains are selected from the group consisting of a CD3 zeta, FcR gamma, FcR beta, CD3 gamma, CD3 delta, CD3 epsilon, CD5, CD22, CD79a, CD79b, CD278 (ICOS), CD66d, DAP10, and a DAP12 intracellular signaling domain.

In some embodiments, at least one of the one or more primary intracellular signaling domains is a CD3 zeta intracellular signaling domain.

In some embodiments, the one or more costimulatory intracellular signaling domains are selected from the group consisting of a CD27, CD28, 4-1 BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, CD83, CDS, ICAM-1, LFA-1 (CD11a/CD18), an MHC class I molecule, BTLA, and a Toll ligand receptor intracellular signaling domain.

In some embodiments, at least one of the one or more co-stimulatory intracellular signaling domains is a CD28 intracellular signaling domain.

In some embodiments, the chimeric antigen receptor includes an N-terminal leader sequence. In some embodiments, the antigen recognition domain includes an N-terminal leader sequence. In some embodiments, the N-terminal leader sequence of the antigen recognition domain is cleaved from the antigen recognition domain during cellular processing and localization of the chimeric antigen receptor to the cellular membrane.

In some embodiments, the autoantigen-binding protein is a multi-chain protein. In some embodiments, the autoantigen-binding protein is a full-length antibody, a dual-variable immunoglobulin domain, a diabody, a triabody, an antibody-like protein scaffold, a Fab fragment, or a F(ab′)₂ molecule.

In some embodiments, the autoantigen is myelin oligodendrocyte glycoprotein, aquaporin 4, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double stranded DNA, single stranded DNA, transferrin, fetuin, factor II, factor VII, fibrin, fibrinogen, C1, C1q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

In another aspect, the present disclosure provides a viral vector that includes a nucleic acid cassette as described herein.

In some embodiments, the viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus. In some embodiments, the viral vector is a Retroviridae family viral vector. In some embodiments, the Retroviridae family viral vector is a lentiviral vector. In some embodiments, the Retroviridae family viral vector is an alpharetroviral vector or a gammaretroviral vector.

In some embodiments, the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

In some embodiments, the viral vector is a pseudotyped viral vector. In some embodiments, the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. In some embodiments, the pseudotyped viral vector is a pseudotyped lentiviral vector.

In some embodiments, the pseudotyped viral vector includes an envelope protein from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the pseudotyped viral vector includes a VSV-G envelope protein.

### BRIEF DESCRIPTION OF THE DRAWINGS

FIGS. 1A and 1B are schematics of lentiviral vector constructs designed to allow expression of chimeric antigen receptors (CAR) under the control of a constitutive promoter for PoC studies. FIG. 1A is a schematic showing basic components of lentiviral construct design. Single chain variable fragments (scFv) were generated by linking heavy and light chain sequences from antibodies with known antigen specificity. A His-tag was introduced to facilitate detection of CARs. Second generation CAR signaling domains were chosen for compatibility with regulatory T cell function. For PoC studies, an scFv (FIG. 1B) with specificity for an irrelevant antigen (Ag) was selected to allow optimisation of in vitro assays and to test the safety and function of CAR biology in vivo. RRE (Rev response element); cPPT (central polypurine tract); EFS (elongation factor 1a short binding sequence); VL (Variable light chain; VH (Variable heavy chain); Woodchuck hepatitis virus post transcriptional regulatory element (WPRE).

FIGS. 2A-2C are a series of graphs demonstrating the expression of an antigen-specific CAR in a human T cell line. Jurkat T cells were transduced with a lentiviral vector (MOI5) to express an antigen-specific CAR (aAg-CAR). FIG. 2A is a set of graphs showing CAR expression, after 72 hours, as assessed by flow cytometry (FC) by incubating cells with 50,000 pg/ml biotinylated CAR ligand (whole protein) before staining with a streptavidin-PE conjugate. FC plots are gated on live Jurkat T cells, depicting untransduced cells (negative control) and transduced cells. A titration of CAR ligand was used to assess receptor expression, quantified as mean fluorescence intensity (MFI). FIG. 2B is a set of graphs showing an MOI titration used to generate a library of Jurkat T cells expressing different levels of Ag-specific CAR. Transgene vector copy number (VCN) (left graph) was measured by ddPCR while % CAR cells was quantified as outlined in (a). FIG. 2C is a graph showing increasing CAR expression with increasing VCN was confirmed by assessing CAR expression by FC, quantified as MFI as a measure of the MOI used.

FIGS. 3A and 3B are graphs demonstrating confirmation of antigen-specific CAR function in vitro in a human T cell line. Transduced Jurkat T cells expressing different levels of aAg-CAR (transduction efficiencies shown in FIGS. 2A-2C) were treated with increasing amounts of CAR ligand in vitro for 24 hrs. FIG. 3A is a set of graphs showing CAR function as assessed by FC analysis of expressed T cell activation markers, CD69 (left graph) and CD25 (right graph), quantified as mean fluorescence intensity (MFI). FIG. 3B is a graph showing the results of an experiment in which supernatants from cultured Jurkat T cells were collected and assessed for IL-2 production by enzyme-linked immunosorbent assay (ELISA). Data points represent mean+/−SEM (n=3).

FIGS. 4A-4D are graphs showing that transduced primary murine T cells express a functional antigen-specific CAR. Purified, CD4⁺CD25⁻ naïve splenic T cells, were activated in vitro using CD3/CD28 microbeads before addition of lentiviral vectors (MOI10) for expression of aAg-CAR. FIG. 4A is a set of graphs showing that, after 72 hours, expression of aAg-CAR was confirmed by FC analysis. Plots are gated on live, CD4⁺ T cells. Untransduced cells were used as negative controls. FIG. 4B shows the % of transduced cells quantified as % of live, CD4⁺ T cells (n=4). FIGS. 4C and 40 show the results of experiments in which transduced CD4⁺CD25⁻ T cells were treated with increasing concentrations of CAR ligand in vitro for 48 hrs. T cell activation was assessed by measuring CD0069 (right graph) and CD25 (left graph) expression by FC, quantified as MFI (FIG. 4C). Supernatants from cultured cells were assessed in parallel for IL-2 secretion. Data points represent mean+/−SEM (n=4) (FIG. 4D).

FIGS. 5A and 5B are graphs showing the transduced primary murine regulatory T cells secrete the immunosuppressive cytokine, IL-10, following activation of CAR in vitro. Purified, CD4⁺CD25⁺ Tregs, were activated in vitro using CD3/CD28 microbeads before lentiviral transduction (MOI10) for expression aAg-CAR. FIG. 5A is a graph showing that, after 72 hrs, expression of aAg-CAR was confirmed by FC analysis. Plot gated on live, CD4⁺ T cells. FIG. 5B is a graph showing the results of an experiment in which transduced CD4⁺CD25⁺ Tregs were cultured for 48 hrs in media alone or 10 μg CAR ligand. Supernatants were collected and IL-10 secretion quantified. Bars represent mean+/−SEM with individual data points shown (n=4). Statistical significance assessed by unpaired T test: **p=0.0019

FIGS. 6A-6C show that transplantation of transduced murine bone marrow HSC leads to generation of regulatory T cells with preferential FoxP3 promoter directed transgene expression within reconstituted immune compartments. Lineage− BM cells were isolated and transduced with lentiviral constructs designed to express green fluorescent protein (GFP) under the control of a Treg (Foxp3) promoter. 10 weeks post-transplantation, expression of GFP was assessed within the reconstituted immune compartment. FIG. 6A is a schematic showing the Treg promoter design. Conserved non-coding sequence (CNS) domains 1,2 and 3, Foxp3 promoter and 3′UTR sequence elements within the construct are designed to enhance transgene expression within the Treg compartment, while limiting transgene expression within other immune subsets. Promoter activity assessed by expression of GFP. FIG. 6B is a representative FC plot depicting GFP expression profile in CD4⁺ CD25⁺ regulatory T cells derived from the spleen of transplanted animals. FIG. 6C shows the activity of Foxp3-promoter assessed in immune cells indicated by FC. GFP expression was quantified as MFI. Individual data points represent biological replicates with bars representing mean+/−SEM (n=4). BM (bone marrow); DP (double positive); SP (single positive); MLNs (mesenteric lymph nodes); pLNs (peripheral lymph nodes).

FIGS. 7A-7C show that transplantation of transduced murine bone marrow HSC leads to generation of CAR expressing regulatory T cells in vivo. Lineage− BM cells were isolated and transduced with lentiviral constructs to express an antigen-specific CAR (CAR+) or an irrelevant transgene (CAR-) under the control of a Treg (Foxp3) promoter. 10 weeks post-transplantation, CAR expression was assessed throughout the immune compartment. Changes in Treg development and function in bone marrow chimeric mice were measured ex vivo. FIG. 7A is a schematic showing components of Treg promoter design. Promoter activity assessed by expression of antigen-specific CAR. FIG. 7B is a representative FC plot depicting CAR expression profile in CD4⁺ CD25⁺ regulatory T cells derived from spleen of transplanted animals. FIG. 7C is a set of graphs showing that comparable number and phenotype of splenic regulatory T cells expressing a CAR (CAR+) or irrelevant transgene (CAR−) are detected, assessed by ex vivo FC analysis. Total number of regulatory cells per spleen quantified (left graph). Expression levels of key regulatory T cell genes including the transcription factor Foxp3 and surface marker CD25 are quantified as MFI (middle and right graph), respectively. Individual data points represent biological replicates with bars representing mean+/−SEM (CAR−n=4; CAR+n=6). Statistical differences were assessed by unpaired T cell test with no significant differences detected.

FIGS. 8A and 8B show that transduced murine bone marrow HSC derived Tregs expressing CAR have comparable immunosuppressive activity to Tregs expressing an irrelevant transgene. Lineage− BM cells were isolated and transduced with lentiviral constructs to express an antigen-specific CAR (CAR+) or irrelevant transgene (CAR−) under the control of a Treg (Foxp3) promoter. 10 weeks post transplantation, regulatory T cells were isolated from peripheral immune organs and assessed in vitro for changes in immune function. FIG. 8A shows the results of an experiment in which CAR expressing Tregs were assessed for immunosuppressive capacity by culturing Tregs with cell tracer violet labelled effector T cells. Effector T cells were stimulated with CD3/CD28 microbeads for 96 hrs in the presence of control CAR− or Ag-CAR+ Tregs. Representative histograms depict cell tracer dye profiles for experimental conditions indicated. FIG. 8B shows the results of an experiment in which proliferative responses were quantified by dilution of Cell Tracer dye. Data represented as percentage of cell tracer labelled cells that have undergone division, “% Proliferation”. Individual data points represent biological replicates with bars representing mean+/−SEM (CAR− n=4; CAR+n=6). Statistical significance was assessed by paired T test for each comparable ratio of cells with no significant differences detected.

FIGS. 9A-9D show that transduced murine bone marrow HSC derived Tregs can be activated by antigen-specific CAR stimulation, and demonstrate enhanced immunosuppressive potential. Lineage-BM cells were isolated and transduced with lentiviral constructs to express an antigen-specific CAR (CAR+) under the control of a Treg specific (Foxp3) promoter or an irrelevant transgene (control CAR−). 10 weeks post transplantation, regulatory T cells were isolated from peripheral immune organs and cultured in vitro with CAR ligand for 48 hrs to assess activation. FIG. 9A shows representative histograms depict changes in CD25 expression levels following stimulation with 10 μg CAR ligand. CD25 levels are quantified in FIG. 9B on control CAR− and CAR+ expressing regulatory T cells as MFI. FIGS. 9C and 9D show the results of an experiment in which control (black circles) and CAR expressing Tregs (open squares) were exposed to 10 μg CAR ligand in the absence (FIG. 9C) or presence of CD3/CD28 microbeads (FIG. 9D) for 48 hrs. Supernatants were collected and IL10 secretion determined by ELISA. Statistical significance assessed by paired T test with p values shown.

### DEFINITIONS

As used herein, the term “pluripotent cell” refers to a cell that possesses the ability to develop into more than one differentiated cell type. For example, a pluripotent cell may be a pluripotent hematopoietic cell that possesses the ability to develop into more than one differentiated cell type of the hematopoietic lineage, such as granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent hematopoietic cells are ESCs, iPSCs, lymphoid progenitor cells, and CD34+ cells.

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34-. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC can be CD34+, CD38−, CD45RA−, CD90+, CD49F+, and lin− (negative for mature lineage markers including CO2, CD3, CD4, CD7, CD8, CD10, CD11B, CD19, CD20, CD56, CD235A). In mice, bone marrow LT-HSC can be CD34−, SCA-1+, C-kit+, CD135−, Slamf1/CD150+, CD48−, and lin− (negative for mature lineage markers including Ter119, CD11b, Gr1, CD3, CD4, CD8, B220, IL-7ra), whereas ST-HSC can be CD34+, SCA-1+, C-kit+, CD135−, Slamf1/CD150+, and lin− (negative for mature lineage markers including Ter119, CD11b, Gr1, CD3, CD4, CD8, B220, IL-7ra). In addition, ST-HSC are less quiescent (i.e., more active) and more proliferative than L T-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, the terms “hematopoietic progenitor cells” and “HPCs” refer to immature blood cells that have the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of hematopoietic progenitor cells include lymphoid progenitor cells and myeloid progenitor cells.

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability to propagate indefinitely under suitable in vitro culture conditions. ES cells are described, for example, in Thomson et al., Science 282:1145 (1998), the disclosure of which is incorporated herein by reference as it pertains to the structure and functionality of embryonic stem cells.

As used herein, the terms “induced pluripotent stem cell,” “iPS cell,” and “iPSC” refer to a pluripotent stem cell that can be derived directly from a differentiated somatic cell. Human PS cells can be generated by introducing specific sets of reprogramming factors into a non-pluripotent cell that can include, for example, Oct3/4, Sox family transcription factors (e.g., Sox1, Sox2, Sox3, Sox15), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human PS cells can also be generated, for example, by the use of miRNAs, small molecules that mimic the actions of transcription factors, or lineage specifiers. Human PS cells are characterized by their ability to differentiate into any cell of the three vertebrate germ layers, e.g., the endoderm, the ectoderm, or the mesoderm. Human PS cells are also characterized by their ability propagate indefinitely under suitable in vitro culture conditions. Human PS cells are described, for example, in Takahashi and Yamanaka, Cell 126:663 (2006), the disclosure of which is incorporated herein by reference as it pertains to the structure and functionality of PS cells.

As used herein, the term “autologous” refers to cells, tissues, nucleic acid molecules, or other substances obtained or derived from an individual's own cells, tissues, nucleic acid molecules, or the like. For example, in the context of a population of cells (e.g., a population of pluripotent cells) expressing one or more proteins described herein, autologous cells include those that are obtained from the patient undergoing therapy that are then transduced or transfected with a vector that directs the expression of one or more proteins of interest.

As used herein, the term “allogeneic” refers to cells, tissues, nucleic acid molecules, or other substances obtained or derived from a different subject of the same species. For example, in the context of a population of cells (e.g., a population of pluripotent cells) expressing one or more proteins described herein, allogeneic cells include those that are (i) obtained from a subject that is not undergoing therapy and are then (ii) transduced or transfected with a vector that directs the expression of one or more desired proteins. The phrase “directs expression” refers to the inclusion of one or more polynucleotides encoding the one or more proteins to be expressed. The polynucleotide may contain additional sequence motifs that enhances expression of the protein of interest.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and, are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the term “functional potential” as it pertains to a pluripotent cell, such as a hematopoietic stem cell, refers to the functional properties of stem cells which include: 1) multi-potency (which refers to the ability to differentiate into multiple different blood lineages including, but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells); 2) self-renewal (which refers to the ability of stem cells to give rise to daughter cells that have equivalent potential as the mother cell, and further that this ability can repeatedly occur throughout the lifetime of an individual without exhaustion); and 3) the ability of stem cells or progeny thereof to be reintroduced into a transplant recipient whereupon they home to the stem cell niche and re-establish productive and sustained cell growth and differentiation.

As used herein, the terms “ablate,” “ablating,” “ablation,” “condition,” “conditioning,” and the like refer to the depletion of one or more cells in a population of cells in vivo or ex vivo. In some embodiments of the present disclosure, it may be desirable to ablate endogenous cells within a patient (e.g., a patient undergoing treatment for a disease described herein) before administering a therapeutic composition, such as a therapeutic population of cells, to the patient. This can be beneficial, for example, in order to provide newly-administered cells with an environment within which the cells may engraft. Ablation of a population of endogenous cells can be performed in a manner that selectively targets a specific cell type, for example, using antibodies or antibody-drug conjugates that bind to an antigen expressed on the target cell and subsequently engender the killing of the target cell. Additionally or alternatively, ablation may be performed in a non-specific manner using cytotoxins that do not localize to a particular cell type, but are instead capable of exerting their cytotoxic effects on a variety of different cells. Examples of ablation include depletion of at least 5% of cells (e.g., at least 5%, 10%, 15%, 20%, 25%, 30%, 35%, 40%, 45%, 50%, or more) in a population of cells in vivo or in vitro. Quantifying cell counts within a sample of cells can be performed using a variety of cell-counting techniques, such as through the use of a counting chamber, a Coulter counter, flow cytometry, or other cell-counting methods known in the art.

Exemplary agents that can be used to “ablate” a population of cells in a patient (i.e., to “condition” a patient for treatment) in accordance with the compositions and methods of the disclosure include alkylating agents, such as nitrogen mustards (e.g., bendamustine, chlorambucil, cyclophosphamide, ifosfamide, mechlorethamine, or melphalan), nitrosoureas (e.g., carmustine, lomustine, or streptozocin), alkyl sulfonates (e.g., busulfan), triazines (e.g., dacarbazine ortemozolomide), or ethylenimines (e.g., altretamine or thiotepa). In some embodiments, the one or more conditioning agents are non-myeloablative conditioning agents that selectively target and ablate a specific population of endogenous pluripotent cells, such as a population of endogenous CD34+ HSCs or HPCs. For example, the one or more conditioning agents may include cytarabine, antithymocyte globulin, fludarabine, or idarubicin.

As used herein, the terms “condition” and “conditioning” refer to processes by which a subject is prepared for receipt of a transplant containing a population of cells (e.g., a population of pluripotent cells, such as CD34+ cells). Such procedures promote the engraftment of a cell transplant, for example, by selectively depleting endogenous cells (e.g., endogenous CD34+ cells, among others) thereby creating a vacancy which is in turn filled by the exogenous cell transplant. According to the methods described herein, a subject may be conditioned for cell transplant procedure by administration to the subject of one or more agents capable of ablating endogenous cells (e.g., CD34+ cells, among others), radiation therapy, or a combination thereof. Conditioning regimens useful in conjunction with the compositions and methods of the disclosure may be myeloablative or non-myeloablative. Other cell-ablating agents and methods well known in the art (e.g., antibodies and antibody-drug conjugates) may also be used.

As used herein, the term “myeloablative” or “myeloablation” refers to a conditioning regiment that substantially impairs or destroys the hematopoietic system, typically by exposure to a cytotoxic agent or radiation. Myeloablation encompasses complete myeloablation brought on by high doses of cytotoxic agent or total body irradiation that destroys the hematopoietic system.

As used herein, the term “non-myeloablative” or “myelosuppressive” refers to a conditioning regiment that does not eliminate substantially all hematopoietic cells of host origin.

As used herein in the context of hematopoietic stem and/or progenitor cells, the term “mobilization” refers to release of such cells from a stem cell niche where the cells typically reside (e.g., the bone marrow) into peripheral circulation. “Mobilization agents” are agents that are capable of inducing the release of hematopoietic stem and/or progenitor cells from a stem cell niche into peripheral circulation.

As used herein, the term “expansion agent” refers to a substance capable of promoting the proliferation of a given cell type ex vivo. Accordingly, a “hematopoietic stem cell expansion agent” or an “HSC expansion agent” refers to a substance capable of promoting the proliferation of a population of hematopoietic stem cells ex vivo. Hematopoietic stem cell expansion agents include those that effectuate the proliferation of a population of hematopoietic stem cells such that the cells retain hematopoietic stem cell functional potential. Exemplary hematopoietic stem cell expansion agents that may be used in conjunction with the compositions and methods of the disclosure include, without limitation, aryl hydrocarbon receptor antagonists, such as those described in U.S. Pat. Nos. 8,927,281 and 9,580,426, the disclosures of each of which are incorporated herein by reference in their entirety, and, in particular, compound SR1. Additional hematopoietic stem cell expansion agents that may be used in conjunction with the compositions and methods of the disclosure include compound UM-171 and other compounds described in U.S. Pat. No. 9,409,906, the disclosure of which is incorporated herein by reference in its entirety. Hematopoietic stem cell expansion agents further include structural and/or stereoisomeric variants of compound UM-171, such as the compounds described in US 2017/0037047, the disclosure of which is incorporated herein by reference in its entirety. Additional hematopoietic stem cell expansion agents suitable for use in the instant disclosure include histone deacetylase (HDAC) inhibitors, such as trichostatin A, trapoxin, trapoxin A, chlamydocin, sodium butyrate, dimethyl sulfoxide, suberanilohydroxamic acid, m-carboxycinnamic acid bishydroxamide, HC-toxin, Cyl-2, WF-3161, depudecin, and radicicol, among others described, for example, in WO 2000/023567, the disclosure of which is incorporated herein by reference.

As used herein, the term “T cell” refers to a type of lymphocyte that plays a central role in cell-mediated immunity. T cells can be distinguished from other lymphocytes, such as B cells and NK cells, by the presence of a T cell receptor (TCR) on the cell surface. The T cell receptor confers antigen-specificity to the T cell by recognizing antigens that are associated with a self-molecule encoded by genes within the major histocompatibility complex (MHC). The antigen may be displayed together with MHC molecules on the surface of antigen presenting cells (APCs), virus infected cells, etc. There are several subsets of T cells, each having a distinct function (e.g., effector T cells, regulatory T cells, T helper cells, cytotoxic T cells, memory T cells, natural killer T (NKT) cells, mucosal associated invariant T cells (MAITs), and gamma delta T cells (γδ T cells)).

As used herein, the term “regulatory T cells” or “Treg cells” refers to a subpopulation of immunosuppressive T cells that modulate the immune system, maintain tolerance to self-antigens, and prevent autoimmune diseases. For example, Treg cells have the ability to suppress the proliferation and/or effector function of other T cell populations. Treg cells can be distinguished based on their unique surface protein presentation. For example, a Treg cell may be a T cell expressing CD4, CD25, FOXP3, and/or CD17 biomarkers. Treg cells execute their immunosuppressive effects, for example, through IL-2/IL-2 receptor-dependent mechanisms and by production of inhibitory cytokines (e.g., IL-10, IL-35 and TGF-β).

As used herein, the term “autoreactive effector cell” or “autoreactive effector immune cell” refers to a cell that is involved in the promotion of an immune effector response (e.g., promotion of an immune response to a target) and that recognizes an autoantigen. Examples of autoreactive effector immune cells include B cells, T cells, and natural killer (NK) cells.

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “autoantigen-binding protein” refers to a protein (e.g., a single-chain protein or a protein comprised of a plurality of polypeptide subunits) that specifically binds an antigen that is expressed endogenously in a subject (e.g., a mammalian subject, such as a human subject).

Examples of autoantigen-binding proteins are single-chain proteins, such as chimeric antigen receptors and single-chain antibody fragments, that specifically bind an antigen that is expressed endogenously in a subject having an autoimmune disease. Additional examples of autoantigen-binding proteins are multi-chain proteins, such as T cell receptors and full-length antibodies, that specifically bind an antigen that is expressed endogenously in a subject having an autoimmune disease.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, primatized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rIgG, and scFv fragments. Moreover, unless otherwise indicated, the term “monoclonal antibody” (mAb) is meant to include both intact molecules, as well as antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316 (1983); incorporated herein by reference).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment that includes two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426 (1988), and Huston et al., Proc. Natl. Acad. Sci. USA 85:5879-5883 (1988)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may include modifications in these hybrid hypervariable positions. The variable domains of native heavy and light chains each include four framework regions that primarily adopt a p-sheet configuration, connected by three CDRs, which form loops that connect, and in some cases form part of, the p-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al., unless otherwise indicated.

As used herein, the term “variable region CDR” includes amino acids in a CDR or complementarity determining region as identified using sequence or structure-based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616, 1977 and Kabat, et al., Sequences of Proteins of Immunological Interest, Fifth Edition, U.S. Department of Health and Human Services, NIH Publication No. 91-3242, 1991; by Chothia et al., (J. Mol. Biol. 196:901-917, 1987), and by MacCallum et al., (J. Mol. Biol. 262:732-745, 1996) where the definitions include overlapping or subsets of amino acid residues when compared against each other. The term “CDR” may be, for example, a CDR as defined by Kabat based on sequence comparisons.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “hinge region,” in the context of antibodies or antigen-binding fragments thereof, refers to the domain of an antibody or antigen-binding fragment thereof (e.g., an IgG2 antibody or antigen-binding fragment thereof) located between the antigen-binding portion(s) of the antibody or antigen-binding fragment thereof, such as the Fab region of the antibody or antigen-binding fragment thereof, and the portion of the antibody or antigen-binding fragment thereof that dictates the isotype of the antibody or antigen-binding fragment thereof, such as the Fc region of the antibody or antigen-binding fragment thereof. For example, in the context of a monoclonal antibody, the hinge region is the polypeptide situated approximately in the center of each heavy chain, connecting the CH1 domain to the CH2 and CH3 domains. The hinge region of an antibody or antigen-binding fragment thereof may provide a chemical linkage between chains of the antibody or antigen-binding fragment thereof. For instance, in a monoclonal antibody, the cysteine residues within the hinge region form inter-chain disulfide bonds, thereby providing explicit covalent bonds between heavy chains. As used herein, antibody hinge regions are numbered according to the numbering system of Kabat et al, Sequences of Proteins of Immunological Interest (National Institute of Health, Bethesda, Md. 1987), the disclosure of which is incorporated herein by reference.

As used herein, the term “bispecific antibodies” refers to antibodies (e.g., monoclonal, often human or humanized antibodies) that have binding specificities for at least two different antigens. For example, one of the binding specificities can be directed towards an autoantigen (e.g., myelin oligodendrocyte glycoprotein), and the other can be for any other antigen, e.g., for a cell-surface protein, receptor, receptor subunit, tissue-specific antigen, virally derived protein, virally encoded envelope protein, bacterially derived protein, or bacterial surface protein, etc.

As used herein, the term “chimeric” antibody refers to an antibody having variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, or bison, among others). Methods for producing chimeric antibodies are known in the art. See, e.g., Morrison, Science. 229(4719):1202-7 (1985); Oi et al. BioTechniques. 4:214-221 (1986); Gillies et al. J. Immunol. Methods. 125:191-202 (1985); U.S. Pat. Nos. 5,807,715; 4,816,567; and 4,816,397; incorporated herein by reference.

As used herein, the term “diabodies” refers to bivalent antibodies that include two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabodies” refers to trivalent antibodies that include three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structure, peptides configured in this way typically trimerize so as to position the V_{H} and V_{L} domains of neighboring peptide chains spatially proximal to one another to permit proper folding (see Holliger et al., Proc. Natl. Acad. Sci. USA 90:6444-48, 1993; incorporated herein by reference).

As used herein, a “dual variable domain immunoglobulin” (“DVD-Ig”) refers to an antibody that combines the target-binding variable domains of two monoclonal antibodies via linkers to create a tetravalent, dual-targeting single agent. (Gu et al., Meth. Enzymol., 502:25-41, 2012; incorporated by reference herein). Suitable linkers for use in the light chains of the DVDs described herein include those identified on Table 2.1 on page 30 of Gu et al.

As used herein, the term “human antibody” refers to an antibody in which substantially every part of the protein (e.g., CDR, framework, CL, CH domains (e.g., C_{H}1, C_{H}2, C_{H}3), hinge, (VL, VH)) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can include a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will include substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also include at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

As used herein, the term “primatized antibody” refers to an antibody that includes framework regions from primate-derived antibodies and other regions, such as CDRs and/or constant regions, from antibodies of a non-primate source. Methods for producing primatized antibodies are known in the art. See e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “monoclonal antibody” refers to an antibody that is derived from a single clone, including any eukaryotic, prokaryotic, or phage clone, and not the method by which it is produced.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of an scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of an scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

As used herein, the term “chimeric antigen receptor” (“CAR”) refers to a recombinant polypeptide containing one or more antigen recognition regions (e.g., one or more CDRs) that recognize, and specifically bind to, a given antigen (e.g., an autoantigen). CARs, as described herein, generally contain at least an extracellular antigen recognition domain, a hinge domain, a transmembrane domain, and a cytoplasmic signaling domain (also referred to herein as “an intracellular signaling domain”) that includes a functional signaling domain derived from a stimulatory molecule as defined herein. The stimulatory molecule may be the zeta chain associated with the T cell receptor complex. In some embodiments, the intracellular signaling domain further contains one or more functional signaling domains derived from at least one costimulatory molecule, as described below. The costimulatory molecule may contain, for example, 4-1 BB (i.e., CD137), CD27, and/or CD28. In some embodiments, the CAR contains a chimeric fusion protein having an extracellular antigen recognition domain, a hinge domain, a transmembrane domain, and a cytoplasmic signaling domain that includes a functional signaling domain derived from a stimulatory molecule. The CAR may contain, for example, a chimeric fusion protein having an extracellular antigen recognition domain, a hinge domain, a transmembrane domain, and a cytoplasmic signaling domain that includes a functional signaling domain derived from a co-stimulatory molecule and a functional signaling domain derived from a stimulatory molecule. In some embodiments, a CAR contains a chimeric fusion protein having an extracellular antigen recognition domain, a hinge domain, a transmembrane domain, and an intracellular signaling domain that includes two functional signaling domains derived from one or more co-stimulatory molecule(s) and a functional signaling domain derived from a stimulatory molecule. In some embodiments, the CAR contains a chimeric fusion protein having an extracellular antigen recognition domain, a hinge domain, a transmembrane domain, and an intracellular signaling domain that includes at least two functional signaling domains derived from one or more co-stimulatory molecule(s) and a functional signaling domain derived from a stimulatory molecule. A CAR may contain a leader sequence at the amino-terminus of the CAR fusion protein. In some embodiments, a CAR further contains a leader sequence at the N-terminus of the extracellular antigen recognition domain, which may be cleaved from the antigen recognition domain, e.g., (an scFv) during cellular processing and localization of the CAR to the cellular membrane. For the avoidance of doubt, as used herein, the terms “intracellular domain” and “cytoplasmic domain” are used interchangeably.

The term “signaling domain” refers to the functional portion of a protein which acts by transmitting information within the cell to regulate cellular activity via defined signaling pathways by generating second messengers or functioning as effectors by responding to such messengers. A CAR described herein may contain an antibody or antibody fragment thereof, which may exist in a variety of forms. For example, the antigen recognition domain may be expressed as part of a contiguous polypeptide chain including, for example, a single domain antibody fragment (sdAb), a single chain antibody (e.g., an scFv), and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “hinge domain,” in the context of CARs, refers to an extracellular portion of a CAR that plays a role in positioning the antigen recognition domain away from the T cell surface to enable proper cell/cell contact, antigen binding, and activation. A CAR generally includes one or more hinge domains between the antigen recognition domain and the transmembrane domain. Examples of hinge domains include those derived from CD28, CD8 (e.g., CD8a), IgG1/IgG4 (hinge-Fc portion), CD4, CD7, and IgD.

As used herein, the term “transmembrane domain” refers to a portion of a CAR that fuses the extracellular antigen recognition domain and intracellular signaling domain and anchors the CAR to the plasma membrane of the T cell. Examples of transmembrane domains include those derived from CD28, CD3 zeta, CD8 (e.g., CD8α), FcRIγ, CD4, CD7, OX40, and MHC (H2-Kb).

A “stimulatory molecule,” as the term is used herein, refers to a molecule expressed by a T cell that provides the primary cytoplasmic signaling sequence(s) that regulates primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM-containing primary cytoplasmic signaling sequence that may be used in conjunction with the compositions and methods of the disclosure include, but are not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d. In an exemplary CAR molecule of the disclosure, the intracellular signaling domain in any one or more CAR molecules of the disclosure includes an intracellular signaling sequence, e.g., a primary signaling sequence of CD3-zeta. In a specific CAR of the disclosure, the primary signaling sequence of CD3-zeta is the human sequence, or the equivalent residues from a non-human species, e.g., mouse, rodent, monkey, ape and the like.

An “intracellular signaling domain,” as the term is used herein, refers to an intracellular portion of a molecule. The intracellular signaling domain may generate a signal that promotes an immunosuppressive function of the CAR-containing cell, e.g., a CAR Treg cell. An example of an immunosuppressive function, e.g., in a Treg cell, includes suppression of activity and/or proliferation of an autoreactive effector immune cell.

In some embodiments, the intracellular signaling domain can include a primary intracellular signaling domain. Exemplary primary intracellular signaling domains include those derived from the molecules responsible for primary stimulation, or antigen dependent simulation. In an embodiment, the intracellular signaling domain can include a costimulatory intracellular domain. Exemplary costimulatory intracellular signaling domains include those derived from molecules responsible for costimulatory signals, or antigen independent stimulation. For example, in the case of a CAR Treg, a primary intracellular signaling domain can include a cytoplasmic sequence of a T cell receptor, and a costimulatory intracellular signaling domain can include a cytoplasmic sequence from a co-receptor or costimulatory molecule.

A primary intracellular signaling domain can include a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD66d, DAP10 and DAP12.

As used herein “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect, the cytoplasmic domain of zeta includes residues 52 through 164 of GenBank Acc. No. BAG36664.1 or the equivalent residues from a non-human species, e.g., mouse, rodent, monkey, ape and the like, that are functional orthologs thereof.

A “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to, an MHC class I molecule, BTLA and a Toll ligand receptor, as well as OX40, CD27, CD28, CDS, ICAM-1, LFA-1 (CD11a/CD18) and 4-1BB (CD137).

A costimulatory intracellular signaling domain can be derived from the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can include the entire intracellular portion, or the entire native intracellular signaling domain, of the molecule from which it is derived, or a functional fragment thereof.

As used herein, the term “autoimmune disease” refers to a group of diseases resulting from one's own immune system incorrectly attacking one's own tissue. Non-limiting examples of autoimmune disorders include type 1 diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barré, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Ménière's Disease, Mixed Connective Tissue Disease, Multiple Sclerosis, Myasthenia Gravis, Neuromyelitis Optica, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis.

As used herein, the term “inflammation” refers to a signal-mediated response to cellular insult by infectious agents (e.g., pathogens), toxins, tumor cells, irritants and stress. While acute inflammation is important to the defense and protection of body from harmful stimuli (e.g., pathogens, damaged cells, cancer/tumor cells, stress, or irritants), chronic and inappropriately high inflammation can cause tissue destruction (e.g., in autoimmunity, inflammatory diseases, neurodegenerative diseases, or cardiovascular disease). Inflammation represents the consequence of capillary dilation with accumulation of fluid (edema) and the recruitment of leukocytes. For the purpose of use herein, increase or decrease in inflammation is assessed by increase or decrease of leukocyte recruitment, and/or increase or decrease of immune cell activity (e.g., one or more of T cell polarization; T cell activation; dendritic cell activation; neutrophil activation; eosinophil activation; basophil activation; T cell proliferation; B cell proliferation; monocyte proliferation; macrophage proliferation; dendritic cell proliferation; NK cell proliferation; ILC proliferation, mast cell proliferation; neutrophil proliferation; eosinophil proliferation; basophil proliferation; cytotoxic T cell activation; circulating monocytes; peripheral blood hematopoietic stem cells; macrophage polarization; macrophage phagocytosis; macrophage ADCP, neutrophil phagocytosis; monocyte phagocytosis; mast cell phagocytosis; B cell phagocytosis; eosinophil phagocytosis; dendritic cell phagocytosis; macrophage activation; antigen presentation (e.g., dendritic cell, macrophage, and B cell antigen presentation); antigen presenting cell migration (e.g., dendritic cell, macrophage, and B cell migration); lymph node immune cell homing and cell egress (e.g., lymph node homing and egress of T cells, B cells, dendritic cells, or macrophages); NK cell activation; NK cell ADCC, mast cell degranulation; NK cell degranulation; ILC activation, ILC ADCC, ILC degranulation, cytotoxic T cell degranulation; neutrophil degranulation; eosinophil degranulation; basophil degranulation; neutrophil recruitment; eosinophil recruitment; NKT cell activation; B cell activation; regulatory T cell differentiation; dendritic cell maturation; development of HEVs; or development of ectopic or tertiary lymphoid organs (TLOs)). The compositions and methods of the present disclosure may be administered to reduce inflammation in a subject diagnosed as having an autoimmune disease or a subject that does not suffer from an autoimmune disease.

As used herein, the term “leukocyte recruitment” refers to the movement or migration of leukocytes out of the circulatory system and towards the site of tissue damage, infection, injury, or stress. Leukocyte recruitment from the bloodstream to the inflammatory foci within the tissue is fundamental to mounting a successful inflammatory response and forms an essential part of the innate immune response, as evidenced by the recurrent infections and poor survival rate of patients suffering from leukocyte adhesion deficiencies, a class of conditions in which neutrophil trafficking is compromised. Monocytes also use this process in the absence of infection or tissue damage during their development into macrophages. Leukocyte recruitment occurs mainly in post-capillary venules, where molecules that regulate leukocyte trafficking are preferentially expressed. During the process of leukocyte recruitment, leukocytes adhere to the vascular endothelium, and subsequently leave the circulation by transendothelial migration driven by chemoattractants (e.g., chemokines), a process known as diapedesis.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a subject can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the subject. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “nucleic acid cassette” refers to a recombinant nucleic acid (e.g., DNA or cDNA) encoding a gene product (e.g., a gene product described herein). The gene product may be an RNA, peptide, or protein. In addition to the coding region for the gene product, the nucleic acid cassette may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Mantel et al., J. Immunol. 176(6):3593-602 (2006); Lee et al., Exp. Mol. Med. 50(3):e456 (2018); Kim et al., J. Exp. Med. 204(7):1543-51 (2007); Zheng et al., Nature. 463(7282):808-12 (2010); Tone et al., Nat. Immunol. 9(2):194-202 (2008); Dikiy et al., Immunity. 54(5):931-946 (2021); Kawakami et al., Immunity. 54(5):947-961 (2021); and Goeddel, Gene Expression Technology: Methods in Enzymology 185 (Academic Press, San Diego, CA, 1990), the disclosures of which are incorporated by reference in their entirety.

As used herein, the term “lineage-specific” means selective for a particular cell type over another cell type. For example, the term “linage-specific transcription regulatory element” refers to a nucleic acid that controls, at least in part, the transcription of a gene that is found in a particular cell type. Examples of lineage-specific transcription regulatory elements include the Foxp3 promoter, CNS1 enhancer, CNS2 enhancer, CNS3 enhancer, and CNS0 enhancer that control the transcription of the Foxp3 gene, which is a distinct feature of Treg cells.

As used herein, the term “promoter” refers to a recognition site on DNA that is bound by an RNA polymerase. The polymerase drives transcription of the nucleic acid cassette. Exemplary promoters suitable for use with the compositions and methods described herein are described, for example, in Mantel et al., J. Immunol. 176(6):3593-602 (2006); Lee et al., Exp. Mol. Med. 50(3):e456 (2018); Kim et al., J. Exp. Med. 204(7):1543-51 (2007); and Zheng et al., Nature. 463(7282):808-12 (2010). Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of nucleic acid cassettes, vectors, and target cell types.

As used herein, the term “enhancer” refers to a type of regulatory element that can increase the efficiency of transcription regardless of the distance or orientation of the enhancer relative to the transcription start site. Accordingly, enhancers can be placed upstream or downstream of the transcription start site or at a considerable distance from the promoter. Enhancers may also overlap physically and functionally with promoters. A number of polynucleotides that include promoter sequences (e.g., Foxp3 promoter sequences) also contain enhancer sequences (e.g., CNS1 enhancer sequences).

As used herein, the term “Foxp3 promoter” refers to a promoter that turns on transcription of the Foxp3 gene in Treg cells. An exemplary human Foxp3 promoter includes, for example, the nucleic acid set forth in in SEQ ID NO: 1, which is described in Mantel et al., J. Immunol. 176(6):3593-602 (2006). Another example of a human Foxp3 promoter includes the nucleic acid set forth in SEQ ID NO: 2, which is described in Kim et al., J. Exp. Med. 204(7):1543-51 (2007). An exemplary murine Foxp3 promoter includes, for example, the nucleic acid set forth in SEQ ID NO: 3, which is described in Zheng et al., Nature. 463(7282):808-12 (2010). By way of alignment of SEQ ID NO: 3 to the human genome, a further example of a human Foxp3 promoter includes the nucleic acid set forth in SEQ ID NO: 4. Additional examples of Foxp3 promoter nucleic acids include nucleic acids having at least 70% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

As used herein, the term “CNS0 enhancer” refers to an enhancer that increases the transcriptional efficiency of the Foxp3 gene in Treg cells. For example, CNS0 enhancers that may be used in conjunction with the compositions and methods of the disclosure include those that recruit transcription factors Satb1 and/or Stat5. An exemplary murine CNS0 enhancer includes, for example, the nucleic acid set forth in SEQ ID NO: 17, as described in Kawakami et al., Immunity. 54(5):947-961 (2021). By way of alignment of SEQ ID NO: 17 to the human genome, an exemplary human CNS0 enhancer includes, for example, the nucleic acid set forth in SEQ ID NO: 18. Another example of a murine CNS0 enhancer includes the nucleic acid set forth in SEQ ID NO: 19, which is described in Dikiy et al., Immunity. 54(5):931-946 (2021). By way of alignment of SEQ ID NO: 19 to the human genome, a further example of a human CNS0 enhancer includes the nucleic acid set forth in SEQ ID NO: 20. Additional examples of CNS0 enhancer nucleic acids include nucleic acids having at least 70% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

As used herein, the term “CNS1 enhancer” refers to an enhancer that increases the transcriptional efficiency of the Foxp3 gene in Treg cells. For example, CNS1 enhancers that may be used in conjunction with the compositions and methods of the disclosure include those that recruit transcription factors AP-1, NFAT, Smad3, and/or Foxo (e.g., Foxo1 and Foxo3). CNS1 enhancers are thought to contribute to peripheral induction of Treg cells and mucosal immune tolerance. An exemplary human CNS1 enhancer contains, for example, from nucleic acids −500 to +100, with respect to the Foxp3 transcription start site of the human Foxp3 locus, as described in Kim et al., J. Exp. Med. 204(7):1543-51 (2007). An exemplary murine CNS1 enhancer includes, for example, the nucleic acid set forth in SEQ ID NO: 5, which is described in Tone et al., Nat. Immunol. 9(2):194-202 (2008). By way of alignment of SEQ ID NO: 5 to the human genome, an additional example of a human CNS1 enhancer includes the nucleic acid set forth in SEQ ID NO: 6. Another example of a murine CNS1 enhancer includes the nucleic acid set forth in SEQ ID NO: 7, which is described in Zheng et al., Nature. 463(7282):808-12 (2010). By way of alignment of SEQ ID NO: 7 to the human genome, a further example of a human CNS1 enhancer includes the nucleic acid set forth in SEQ ID NO: 8. Additional examples of CNS1 enhancer nucleic acids include nucleic acids having at least 70% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

As used herein, the term “CNS2 enhancer” refers to an enhancer that increases the transcriptional efficiency of the Foxp3 gene in Treg cells. For example, CNS2 enhancers that may be used in conjunction with the compositions and methods of the disclosure include those that recruit transcription factors Runx, Foxp3, Ets-1, CREB, Stat5, NFAT, and/or c-Rel. CNS2 enhancers are highly demethylated in functional Treg cells and are thought to be responsible for the stability of Foxp3 expression in response to T cell receptor stimulation and during Treg cell proliferation. An exemplary human CNS2 enhancer contains, for example, from nucleic acids +2,022 to +2,721, with respect to the Foxp3 transcription start site of the human Foxp3 locus, as described in Kim et al., J. Exp. Med. 204(7):1543-51 (2007). An exemplary murine CNS2 enhancer includes, for example, the nucleic acid set forth in SEQ ID NO: 9, which is described in Kawakami et al., Immunity. 54(5):947-961 (2021). By way of alignment of SEQ ID NO: 9 to the human genome, an additional example of a human CNS2 enhancer includes the nucleic acid set forth in SEQ ID NO: 10. Another example of a murine CNS2 enhancer includes the nucleic acid set forth in SEQ ID NO: 11, which is described in Zheng et al., Nature. 463(7282):808-12 (2010). By way of alignment of SEQ ID NO: 11 to the human genome, a further example of a human CNS2 enhancer includes the nucleic acid set forth in SEQ ID NO: 12. Additional examples of CNS2 enhancer nucleic acids include nucleic acids having at least 70% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

As used herein, the term “CNS3 enhancer” refers an enhancer that increases the transcriptional efficiency of the Foxp3 gene in Treg cells. For example, CNS3 enhancers that may be used in conjunction with the compositions and methods of the disclosure include those that recruit transcription factors Foxo (e.g., Foxo1 and Foxo3) and/or c-Rel. CNS3 enhancers are thought to play a role in thresholding TCR stimuli required for Foxp3 expression and to be important for peripheral and thymic Treg cell generation. An exemplary human CNS3 enhancer contains, for example, from nucleic acids +4,301 to +4,500 with respect to the Foxp3 transcription start site of the human Foxp3 locus, as described in Kim et al., J. Exp. Med. 204(7):1543-51 (2007). An exemplary murine CNS3 enhancer includes, for example, the nucleic acid set forth in SEQ ID NO: 13, which is described in Kawakami et al., Immunity. 54(5):947-961 (2021). By way of alignment of SEQ ID NO: 13 to the human genome, an additional example of a human CNS3 enhancer includes the nucleic acid set forth in SEQ ID NO: 14. Another example of a murine CNS3 enhancer includes the nucleic acid set forth in SEQ ID NO: 15, which is described in Zheng et al., Nature. 463(7282):808-12 (2010). By way of alignment of SEQ ID NO: 15 to the human genome, a further example of a human CNS3 enhancer includes the nucleic acid set forth in SEQ ID NO: 16. Additional examples of CNS3 enhancer nucleic acids include nucleic acids having at least 70% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

As used herein, the term “regulatory sequence” includes promoters, enhancers and other expression control elements (e.g., polyadenylation signals) that control the transcription or translation of the gene(s). Such regulatory sequences are described, for example, in Perdew et al., Regulation of Gene Expression (Humana Press, New York, NY, (2014)); incorporated herein by reference.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “inhibitor” refers to an agent (e.g., a small molecule, peptide fragment, protein, antibody, or antigen-binding fragment thereof) that binds to, and/or otherwise suppresses the activity of, a target molecule.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the terms “transduction” and “transduce” refer to a method of introducing a viral vector construct or a part thereof into a cell and subsequent expression of a nucleic acid cassette encoded by the vector construct or part thereof in the cell.

As used herein, the term “poloxamer” refers to a non-ionic triblock copolymer composed of a central hydrophobic chain of polyoxypropylene flanked by two hydrophilic chains of polyoxyethylene.

Poloxamers are also known by the trade name of “Pluronics” or “Synperonics” (BASF). The block copolymer can be represented by the following formula: HO(C₂H₄O)ₓ(C₃H₆O)_{y}(C₂H₄O)_{z}H. The lengths of the polymer blocks can be customized. As a result, many different poloxamers exist. Poloxamers suitable for use in conjunction with the compositions and methods of the present disclosure include those having an average molecular weight of at least about 10,000 g/mol, at least about 11,400 g/mol, at least about 12,600 g/mol, at least about 13,000 g/mol, at least about 14,600 g/mol, or at least about 15,000 g/mol. Since the synthesis of block copolymers is associated with a natural degree of variation from one batch to another, the numerical values recited above (and those used herein to characterize a given poloxamer) may not be precisely achievable upon synthesis, and the average value will differ to a certain extent. Thus, the term “poloxamer” as used herein can be used interchangeably with the term “poloxamers” (representing an entity of several poloxamers, also referred to as mixture of poloxamers) if not explicitly stated otherwise. The term “average” in relation to the number of monomer units or molecular weight of (a) poloxamer(s) as used herein is a consequence of the technical inability to produce poloxamers all having the identical composition and thus the identical molecular weight. Poloxamers produced according to state-of-the-art methods will be present as a mixture of poloxamers each showing a variability as regards their molecular weight, but the mixture as a whole averaging the molecular weight specified herein. BASF and Sigma Aldrich are suitable sources of poloxamers for use in conjunction with the compositions and methods of the disclosure.

As used herein, for example, in the context of a protein kinase C (PKC) inhibitor, such as staurosporine, the term “variant” refers to an agent containing one or more modifications relative to a reference agent and that (i) retains a functional property of the reference agent (e.g., the ability to inhibit PKC activity) and/or (ii) is converted within a cell (e.g., a cell of a type described herein, such as a CD34+ cell) into the reference agent. In the context of small molecule PKC inhibitors, such as staurosporine, structural variants of a reference compound include those that differ from the reference compound by the inclusion and/or location of one or more substituents, as well as variants that are isomers of a reference compound, such as structural isomers (e.g., regioisomers) or stereoisomers (e.g., enantiomers or diastereomers), as well as prodrugs of a reference compound. In the context of an interfering RNA molecule, a variant may contain one or more nucleic acid substitutions relative to a parent interfering RNA molecule.

As used herein, an agent that inhibits histone deacetylation refers to a substance or composition (e.g., a small molecule, protein, interfering RNA, messenger RNA, or other natural or synthetic compound, or a composition such as a virus or other material composed of multiple substances) capable of attenuating or preventing the activity of histone deacetylase, more particularly its enzymatic activity either via direct interaction or via indirect means such as by causing a reduction in the quantity of a histone deacetylase produced in a cell or by inhibition of the interaction between a histone deacetylase and an acetylated histone substrate. Inhibiting histone deacetylase enzymatic activity means reducing the ability of a histone deacetylase to catalyze the removal of an acetyl group from a histone residue (e.g., a mono-, di-, or tri-methylated lysine residue; a monomethylated arginine residue, or a symmetric/asymmetric dimethylated arginine residue, within a histone protein). Preferably, such inhibition is specific, such that the agent that inhibits histone deacetylation reduces the ability of a histone deacetylase to remove an acetyl group from a histone residue at a concentration that is lower than the concentration of the inhibitor that is required to produce another, unrelated biological effect.

As used herein, the terms “histone deacetylase” and “HDAC” refer to any one of a family of enzymes that catalyze the removal of acetyl groups from the F-amino groups of lysine residues at the N-terminus of a histone. Unless otherwise indicated by context, the term “histone” is meant to refer to any histone protein, including HI, H2A, H2B, H3, H4, and H5, from any species. Human HDAC proteins or gene products, include, but are not limited to, HDAC-1, HDAC-2, HDAC-3, HDAC-4, HDAC-5, HDAC-6, HDAC-7, HDAC-8, HDAC-9, HDAC-10, and HDAC-11.

As used herein, a compound that “activates prostaglandin E receptor signaling” or the like refers to a compound having the ability to increase signal transduction activity of a prostaglandin E receptor in a prostaglandin E receptor-expressing cell that is contacted with the specified compound as compared to prostaglandin E receptor signal transduction activity in a prostaglandin E receptor-expressing cell that is not contacted with the specified compound. Assays that can be used to measure prostaglandin E receptor signal transduction are described, e.g., in WO 2010/108028, the disclosure of which is incorporated herein by reference as it pertains to methods of assessing prostaglandin E receptor signaling.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026, incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Vectors that can be used for the expression of a protein or proteins described herein include plasmids that contain regulatory sequences, such as promoter and enhancer regions, which direct gene transcription. Additionally, useful vectors for expression of a protein or proteins described herein may contain polynucleotide sequences that enhance the rate of translation of the corresponding gene or genes or improve the stability or nuclear export of the mRNA that results from gene transcription. Examples of such sequence elements are 5′ and 3′ untranslated regions, an IRES, and a polyadenylation signal site in order to direct efficient transcription of a gene or genes carried on an expression vector. Expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin, among others.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “subject” and “patient” are used interchangeably and refer to an organism (e.g., a mammal, such as a human) that is at risk of developing or has been diagnosed as having, and/or is undergoing treatment for, a disease, such as an autoimmune disease as described herein.

As used herein, the terms “administering,” “administration,” and the like refer to directly giving a patient a therapeutic agent (e.g., a population of cells, such as a population of pluripotent cells (e.g., embryonic stem cells, induced pluripotent stem cells, or CD34+ cells)) by any effective route. Exemplary routes of administration are described herein and include systemic administration routes, such as intravenous injection, among others.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to or at risk of developing the condition or disorder, as well as those in which the condition or disorder is to be prevented.

As used herein, the term “pharmaceutical composition” refers to a composition containing a therapeutic agent (e.g., a population of cells, such as a population of pluripotent hematopoietic cells (e.g., embryonic stem cells, induced pluripotent stem cells, lymphoid progenitor cells, or CD34+ cells)) that may be administered to a subject, such as a mammal, e.g., a human, in order to prevent, treat or control a particular disease or condition affecting the mammal, such as an autoimmune disease as described herein.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject. The term sample can also relate to a prepared or processed samples, such as a mRNA- or cDNA-containing sample.

As used herein, the term “about” refers to a quantity that varies by as much as 30% (e.g., 25%, 20%, 15%, 10%, 9%, 8%, 7%, 6%, 5%, 4%, 3%, 2% or 1%) relative to a reference quantity.

As used herein, the term “alkyl” refers to monovalent, optionally branched alkyl groups, such as those having from 1 to 6 carbon atoms, or more. This term is exemplified by groups such as methyl, ethyl, n-propyl, isopropyl, n-butyl, isobutyl, tert-butyl, n-hexyl and the like.

As used herein, the term “lower alkyl” refers to alkyl groups having from 1 to 6 carbon atoms.

As used herein, the term “aryl” refers to an unsaturated aromatic carbocyclic group of from 6 to 14 carbon atoms having a single ring (e.g., phenyl) or multiple condensed rings (e.g., naphthyl).

Preferred aryl include phenyl, naphthyl, phenanthrenyl and the like.

As used herein, the terms “aralkyl” and “aryl alkyl” are used interchangeably and refer to an alkyl group containing an aryl moiety. Similarly, the terms “aryl lower alkyl” and the like refer to lower alkyl groups containing an aryl moiety.

As used herein, the term “alkyl aryl” refers to alkyl groups having an aryl substituent, including benzyl, phenethyl and the like.

As used herein, the term “heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, I,3,4-oxadiazolyl, I,3,4-triazinyl, 1,2,3-triazinyl, benzofuryl, [2,3-dihydrojbenzofuryl, isobenzofuryl, benzothienyl, benzotriazolyl, isobenzothienyl, indolyl, isoindolyl, 3H-indolyl, benzimidazolyl, imidazo[I,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “alkyl heteroaryl” refers to alkyl groups having a heteroaryl substituent, including 2-furylmethyl, 2-thienylmethyl, 2-(1H-indol-3-yl)ethyl and the like.

As used herein, the term “lower alkenyl” refers to alkenyl groups preferably having from 2 to 6 carbon atoms and having at least 1 or 2 sites of alkenyl unsaturation. Exemplary alkenyl groups are ethenyl (—CH═CH₂), n-2-propenyl (allyl, —CH₂CH═CH₂) and the like.

As used herein, the term “alkenyl aryl” refers to alkenyl groups having an aryl substituent, including 2-phenylvinyl and the like.

As used herein, the term “alkenyl heteroaryl” refers to alkenyl groups having a heteroaryl substituent, including 2-(3-pyridinyl)vinyl and the like.

As used herein, the term “lower alkynyl” refers to alkynyl groups preferably having from 2 to 6 carbon atoms and having at least 1-2 sites of alkynyl unsaturation, preferred alkynyl groups include ethynyl (—C═CH), propargyl (—CH₂C═CH), and the like.

As used herein, the term “alkynyl aryl” refers to alkynyl groups having an aryl substituent, including phenylethynyl and the like.

As used herein, the term “alkynyl heteroaryl” refers to alkynyl groups having a heteroaryl substituent, including 2-thienylethynyl and the like.

As used herein, the term “cycloalkyl” refers to a monocyclic cycloalkyl group having from 3 to 8 carbon atoms, such as cyclopropyl, cyclobutyl, cyclopentyl, cyclohexyl, cycloheptyl, cyclooctyl, and the like.

As used herein, the term “lower cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

As used herein, the term “heterocycloalkyl” refers to a cycloalkyl group in which one or more ring carbon atoms are replaced with a heteroatom, such as a nitrogen atom, an oxygen atom, a sulfur atom, and the like. Exemplary heterocycloalkyl groups are pyrrolidinyl, piperidinyl, oxopiperidinyl, morpholinyl, piperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the term “alkyl cycloalkyl” refers to alkyl groups having a cycloalkyl substituent, including cyclohexylmethyl, cyclopentylpropyl, and the like.

As used herein, the term “alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

As used herein, the term “carboxy” refers to the group —C(O)OH.

As used herein, the term “alkyl carboxy” refers to C₁-C₅-alkyl groups having a carboxy substituent, including 2-carboxyethyl and the like.

As used herein, the term “acyl” refers to the group —C(O)R, wherein R may be, for example, C₁-C₆-alkyl, aryl, heteroaryl, C₁-C₆-alkyl aryl, or C₁-C₆-alkyl heteroaryl, among other substituents.

As used herein, the term “acyloxy” refers to the group —OC(O)R, wherein R may be, for example, C₁-C₆-alkyl, aryl, heteroaryl, C₁-C₆-alkyl aryl, or C₁-C₆-alkyl heteroaryl, among other substituents.

As used herein, the term “alkoxy” refers to the group —O—R, wherein R is, for example, an optionally substituted alkyl group, such as an optionally substituted C₁-C₆-alkyl, aryl, heteroaryl, C₁-C₆-alkyl aryl, or C₁-C₆-alkyl heteroaryl, among other substituents. Exemplary alkoxy groups include by way of example, methoxy, ethoxy, phenoxy, and the like.

As used herein, the term “alkoxycarbonyl” refers to the group —C(O)OR, wherein R is, for example, hydrogen, C1-C₆-alkyl, aryl, heteroaryl, C₁-C₆-alkyl aryl, or C1-C₆-alkyl heteroaryl, among other possible substituents.

As used herein, the term “alkyl alkoxycarbonyl” refers to alkyl groups having an alkoxycarbonyl substituent, including 2-(benzyloxycarbonyl)ethyl and the like.

As used herein, the term “aminocarbonyl” refers to the group —C(O)NRR′, wherein each of R and R′ may independently be, for example, hydrogen, C1-C₆-alkyl, aryl, heteroaryl, C₁-C₆-alkyl aryl, or C₁-C₆-alkyl heteroaryl, among other substituents.

As used herein, the term “alkyl aminocarbonyl” refers to alkyl groups having an aminocarbonyl substituent, including 2-(dimethylaminocarbonyl)ethyl and the like.

As used herein, the term “acylamino” refers to the group —NRC(O)R′, wherein each of R and R′ may independently be, for example, hydrogen, C1-C₆-alkyl, aryl, heteroaryl, C₁-C₆-alkyl aryl, or C₁-C₆-alkyl heteroaryl, among other substituents.

As used herein, the term “alkyl acylamino” refers to alkyl groups having an acylamino substituent, including 2-(propionylamino)ethyl and the like.

As used herein, the term “ureido” refers to the group —NRC(O)NR′R″, wherein each of R, R′, and R″ may independently be, for example, hydrogen, C₁-C₆-alkyl, aryl, heteroaryl, C₁-C₆-alkyl aryl, C₁-C₆-alkyl heteroaryl, cycloalkyl, or heterocycloalkyl, among other substituents. Exemplary ureido groups further include moieties in which R′ and R″, together with the nitrogen atom to which they are attached, form a 3-8-membered heterocycloalkyl ring.

As used herein, the term “alkyl ureido” refers to alkyl groups having an ureido substituent, including 2-(N′-methylureido)ethyl and the like.

As used herein, the term “amino” refers to the group —NRR′, wherein each of R and R′ may independently be, for example, hydrogen, C₁-C₆-alkyl, aryl, heteroaryl, C₁-C₆-alkyl aryl, C₁-C₆-alkyl heteroaryl, cycloalkyl, or heterocycloalkyl, among other substituents. Exemplary amino groups further include moieties in which R and R′, together with the nitrogen atom to which they are attached, can form a 3-8-membered heterocycloalkyl ring.

As used herein, the term “alkyl amino” refers to alkyl groups having an amino substituent, including 2-(1-pyrrolidinyl)ethyl and the like.

As used herein, the term “ammonium” refers to a positively charged group —N⁺RR′R″, wherein each of R, R′, and R″ may independently be, for example, C₁-C₆-alkyl, C₁-C₆-alkyl aryl, C₁-C₆-alkyl heteroaryl, cycloalkyl, or heterocycloalkyl, among other substituents. Exemplary ammonium groups further include moieties in which R and R′, together with the nitrogen atom to which they are attached, form a 3-8-membered heterocycloalkyl ring.

As used herein, the term “halogen” refers to fluoro, chloro, bromo and iodo atoms.

As used herein, the term “sulfonyloxy” refers to a group —OSO₂—R wherein R is selected from hydrogen, C₁-C₆-alkyl, C₁-C₆-alkyl substituted with halogens, e.g., an —OSO₂—CF₃ group, aryl, heteroaryl, C₁-C₆-alkyl aryl, and C₁-C₆-alkyl heteroaryl.

As used herein, the term “alkyl sulfonyloxy” refers to alkyl groups having a sulfonyloxy substituent, including 2-(methylsulfonyloxy)ethyl and the like.

As used herein, the term “sulfonyl” refers to group “—SO₂—R” wherein R is selected from hydrogen, aryl, heteroaryl, C₁-C₆-alkyl, C₁-C₆-alkyl substituted with halogens, e.g., an —SO₂—CF₃ group, C₁-C₆-alkyl aryl or C₁-C₆-alkyl heteroaryl.

As used herein, the term “alkyl sulfonyl” refers to alkyl groups having a sulfonyl substituent, including 2-(methylsulfonyl)ethyl and the like.

As used herein, the term “sulfinyl” refers to a group “—S(O)—R” wherein R is selected from hydrogen, C₁-C₆-alkyl, C₁-C₆-alkyl substituted with halogens, e.g., a —SO—CF₃ group, aryl, heteroaryl, C₁-C₆-alkyl aryl or C₁-C₆-alkyl heteroaryl.

As used herein, the term “alkyl sulfinyl” refers to C₁-C₅-alkyl groups having a sulfinyl substituent, including 2-(methylsulfinyl)ethyl and the like.

As used herein, the term “sulfanyl” refers to groups —S—R, wherein R is, for example, alkyl, aryl, heteroaryl, C₁-C₆-alkyl aryl, or C₁-C₆-alkyl heteroaryl, among other substituents. Exemplary sulfanyl groups are methylsulfanyl, ethylsulfanyl, and the like.

As used herein, the term “alkyl sulfanyl” refers to alkyl groups having a sulfanyl substituent, including 2-(ethylsulfanyl)ethyl and the like.

As used herein, the term “sulfonylamino” refers to a group —NRSO₂—R′, wherein each of R and R′ may independently be hydrogen, C₁-C₆-alkyl, aryl, heteroaryl, C₁-C₆-alkyl aryl, or C₁-C₆-alkyl heteroaryl, among other substituents.

As used herein, the term “alkyl sulfonylamino” refers to alkyl groups having a sulfonylamino substituent, including 2-(ethylsulfonylamino)ethyl and the like.

Unless otherwise constrained by the definition of the individual substituent, the above set out groups, like “alkyl”, “alkenyl”, “alkynyl”, “aryl” and “heteroaryl” etc. groups can optionally be substituted, for example, with one or more substituents, as valency permits, such as a substituent selected from alkyl (e.g., C₁-C₆-alkyl), alkenyl (e.g., C₂-C₆-alkenyl), alkynyl (e.g., C₂-C₆-alkynyl), cycloalkyl, heterocycloalkyl, alkyl aryl (e.g., C₁-C₆-alkyl aryl), alkyl heteroaryl (e.g., C₁-C₆-alkyl heteroaryl, alkyl cycloalkyl (e.g., C₁-C₆-alkyl cycloalkyl), alkyl heterocycloalkyl (e.g., C₁-C₆-alkyl heterocycloalkyl), amino, ammonium, acyl, acyloxy, acylamino, aminocarbonyl, alkoxycarbonyl, ureido, aryl, heteroaryl, sulfinyl, sulfonyl, alkoxy, sulfanyl, halogen, carboxy, trihalomethyl, cyano, hydroxy, mercapto, nitro, and the like. In some embodiments, the substitution is one in which neighboring substituents have undergone ring closure, such as situations in which vicinal functional substituents are involved, thus forming, e.g., lactams, lactones, cyclic anhydrides, acetals, thioacetals, and aminals, among others.

As used herein, the term “optionally fused” refers to a cyclic chemical group that may be fused with a ring system, such as cycloalkyl, heterocycloalkyl, aryl, or heteroaryl. Exemplary ring systems that may be fused to an optionally fused chemical group include, e.g., indolyl, isoindolyl, benzofuranyl, isobenzofuranyl, benzothiophenyl, benzoxazolyl, benzothiazolyl, benzoisoxazolyl, benzoisothiazolyl, indazolyl, benzimidazolyl, quinolinyl, isoquinolinyl, phthalazinyl, quinoxalinyl, quinazolinyl, cinnolinyl, indolizinyl, naphthyridinyl, pteridinyl, indanyl, naphtyl, 1,2,3,4-tetrahydronaphthyl, indolinyl, isoindolinyl, 2,3,4,5-tetrahydrobenzo[b]oxepinyl, 6,7,8,9-tetrahydro-5H-benzocycloheptenyl, chromanyl, and the like.

As used herein, the term “pharmaceutically acceptable salt” refers to a salt, such as a salt of a compound described herein, that retains the desired biological activity of the non-ionized parent compound from which the salt is formed. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. The compounds can also be administered as pharmaceutically acceptable quaternary salts, such as quaternary ammonium salts of the formula —NR,R′,R″ ⁺Z—, wherein each of R, R′, and R″ may independently be, for example, hydrogen, alkyl, benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, or the like, and Z is a counterion, such as chloride, bromide, iodide, —O-alkyl, toluenesulfonate, methyl sulfonate, sulfonate, phosphate, carboxylate (such as benzoate, succinate, acetate, glycolate, maleate, malate, fumarate, citrate, tartrate, ascorbate, cinnamoate, mandeloate, and diphenylacetate), or the like.

The structural compositions described herein also include the tautomers, geometrical isomers (e.g., E/Z isomers and cis/trans isomers), enantiomers, diastereomers, and racemic forms, as well as pharmaceutically acceptable salts thereof. Such salts include, e.g., acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

As used herein, chemical structural formulas that do not depict the stereochemical configuration of a compound having one or more stereocenters will be interpreted as encompassing any one of the stereoisomers of the indicated compound, or a mixture of one or more such stereoisomers (e.g., any one of the enantiomers or diastereomers of the indicated compound, or a mixture of the enantiomers (e.g., a racemic mixture) or a mixture of the diastereomers). As used herein, chemical structural formulas that do specifically depict the stereochemical configuration of a compound having one or more stereocenters will be interpreted as referring to the substantially pure form of the particular stereoisomer shown. “Substantially pure” forms refer to compounds having a purity of greater than 85%, such as a purity of from 85% to 99%, 85% to 99.9%, 85% to 99.99%, or 85% to 100%, such as a purity of 85%, 86%, 87%, 88%, 89%, 90%, 91%, 92%, 93%, 94%, 95%, 96%, 97%, 98%, 99%, 99.9%, 99.99%, 99.999%, or 100%, as assessed, for example, using chromatography and nuclear magnetic resonance techniques known in the art.

### DETAILED DESCRIPTION

The present disclosure provides compositions and methods for treating autoimmune diseases, such as type 1 diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barré, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Ménière's Disease, Mixed Connective Tissue Disease, Multiple Sclerosis, Myasthenia Gravis, Neuromyelitis Optica, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis, among others. In accordance with the compositions and methods of the disclosure, a patient (e.g., a human patient) may be administered a population of pluripotent cells (e.g., pluripotent hematopoietic cells) that include a nucleic acid cassette that encodes an autoantigen-binding protein. The nucleic acid cassette may be operably linked to one or more lineage-specific transcription regulatory elements that are active in CD4+CD25+ regulatory T (Treg) cells so as to treat or prevent an autoimmune disease, such as one or more of the foregoing conditions. In the context of therapeutic treatment, the pluripotent hematopoietic cells may be administered to the patient to alleviate one or more symptoms of the disease and/or to remedy an underlying molecular pathology associated with the disease, such as to suppress activity and/or proliferation of a population of autoreactive effector immune cells, induce apoptosis of an autoreactive effector immune cell, protect endogenous tissue from an autoimmune response, or reduce inflammation.

The compositions and methods of the disclosure provide significant advantages relative to current methods of treating autoimmune diseases using Treg cell therapies. To date, human polyclonal Treg cells have been used in clinical trials to treat autoimmune diseases. However, polyclonal Treg cell therapy has proven to be ineffective due to a lack of in vivo expansion and persistence of Treg cells as well as a lack of specificity of Treg cells to target tissues. To overcome the lack of specificity of polyclonal Treg cells, Treg cells have been genetically engineered to express receptors, including chimeric antigen receptors or antigen-specific T cell receptors, that can recognize a particular antigen. Despite these advances, one of the hindrances that has been associated with the use of current Treg cell therapies to treat autoimmune diseases is the durability of Treg cells administered directly to a patient. Current research suggests that the cells may last only 3-5 years in vivo, which is of particular concern in the context of chronic autoimmune diseases. The compositions and methods of the disclosure improve upon the existing paradigms for using genetically engineered antigen-specific Treg cells to treat autoimmune diseases by combining the specific suppressive potential of Treg cells with the proven durability of hematopoietic stem cell gene therapy. In particular, the compositions and methods of the disclosure provide pluripotent hematopoietic cells that are genetically engineered to include an antigen-binding protein that, upon differentiation of the hematopoietic cells in vivo, is preferentially expressed in Treg cells. While pluripotent hematopoietic cells can differentiate into mature blood cells of diverse lineages, the antigen-binding protein is specifically expressed in Treg cells due to the expression of lineage-specific transcription regulatory elements (e.g., a Foxp3 promoter) that are preferentially active in CD4+CD25+ regulatory Treg cells.

The compositions and methods of the present disclosure may also impart improved stability to Treg cells by providing tissue-specific regulation of autoantigen binding-protein expression. Expression of the autoantigen-binding protein is, therefore, responsive to the Treg cell phenotype. In contrast, antigen-specific Treg cells administered directly to a patient may lose lineage-specific transcription regulatory elements that are active in Treg cells and become effector T cells. Thus, direct administration of Treg cells to a patient suffering from an autoimmune disease is associated with a risk of further activating an immune response rather than suppressing an immune response.

The compositions and methods of the present disclosure may also provide advantages with respect to manufacturing and feasibility. Direct Treg cell therapy requires multi-parameter cell sorting for large quantities of cells, as there is currently no single marker of Treg cells, leading to significant manufacturing challenges. Additionally, patients with autoimmune diseases have fewer and poorly functioning Treg cells, leading to significant manufacturing challenges for autologous Treg cell therapies.

Hematopoietic stem cells, as provided by the compositions and methods of the present disclosure, have a clear manufacturing protocol and good manufacturing practice.

Furthermore, while hematopoietic stem cell dosages are well-established, effective Treg cell dosages remain unclear and will likely be different for each disease. Long term efficacy of Treg cells administered directly to a patient may also require multiple doses and consequently multiple conditioning regimens.

### Methods of Treating Autoimmune Diseases

Autoimmune diseases are the result of an inappropriate attack of one's own immune system on one's own tissue. These diseases are mediated by T- and B-lymphocytes that incorrectly exhibit reactivity against self-antigens. Regulatory T (Treg) cells have evolved in order to inhibit the activity of immune cells that are cross-reactive with “self” major histocompatability complex (MHC) proteins and other benign antigens, thereby modulating the immune system, maintaining tolerance to self-antigens, and preventing autoimmune diseases. Treg cells represent a heterogeneous class of T-cells that can be distinguished based on their unique surface protein presentation. The most well-understood populations of Treg cells include CD4+, CD25+, FoxP3+ Treg cells and CD17+ Treg cells. The precise mechanisms by which Treg cells mediate suppression of autoreactive effector immune cells (e.g., effector T cells, B cells, and NK cells) is the subject of ongoing investigations, though Treg suppressive function is thought to occur via contact-dependent cell-to-cell crosstalk mechanisms and via the secretion of inhibitory cytokines, such as IL-10, IL-35, and TGF-β. It has also been shown that certain classes of Treg cells inhibit production of the proliferation-inducing cytokine IL-2 in target T-cells and may additionally sequester IL-2 from autoreactive cells by virtue of the affinity of CD25 (a subdomain of the IL-2 receptor) for IL-2. Moreover, it has been shown that CD4+, CD25+, FoxP3+ Treg cells are also present in B-cell-rich areas and are capable of directly suppressing immunoglobulin production independent of their ability to attenuate T_{H}2-cell activity.

Although Treg cell therapy has been investigated as a potential therapeutic paradigm for autoimmune diseases, one problem with Treg cell therapies is that Treg cells are prone to losing their phenotype (e.g., CD25+ phenotype). Therefore, Treg cells can lose their suppressive functions and convert to autoreactive effector immune cells (e.g., effector T cells), resulting in the activation of an immune response and the worsening of an autoimmune disease.

The compositions and methods of the disclosure offer a solution to this problem by providing pluripotent cells, such as pluripotent hematopoietic cells (e.g., HSCs), that can differentiate into diverse cells of the hematopoietic lineage for the treatment of autoimmune diseases. For example, pluripotent hematopoietic cells may differentiate into granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). The pluripotent hematopoietic cells described herein include a nucleic acid cassette encoding an autoantigen-binding protein that provides localization to target tissues. Although pluripotent hematopoietic cells can differentiate into multiple cell types of the hematopoietic lineage, expression of the autoantigen-binding protein is restricted to cells that differentiate into Treg cells. Treg-specific expression of the autoantigen-binding protein is achieved by placing the nucleic acid cassette encoding the autoantigen-binding protein under the control of transcription regulatory elements that are preferentially active in CD4+CD25+ Treg cells. The autoantigen-binding protein can direct Treg cells to autoantigens present at sites of autoimmunity, thereby focusing Treg suppressor functions at these sites to treat an autoimmune disease.

The advantage of delivering pluripotent hematopoietic cells (e.g., HSCs) to a patient (e.g., a human patient suffering from an autoimmune disease) that are upstream of differentiated Treg cells is that HSC-derived Treg cells will cease to express the autoantigen-binding protein if the Treg cells are converted to autoreactive effector immune cells (e.g., effector T cells) due to CD4+CD25+ Treg-specific transcription regulatory elements that control the expression of the autoantigen-binding protein. In contrast, Treg cells that express an autoantigen-binding protein and that are delivered directly to a patient (e.g., a human patient suffering from an autoimmune disease) could lose their phenotype and convert to autoreactive effector immune cells that continue to express the autoantigen-binding protein. Autoreactive effector immune cells (e.g., effector T cells) expressing an autoantigen-binding protein would be directed to sites of autoimmunity, leading to the activation of an immune response and the worsening of the autoimmune disease. Therefore, the compositions and methods of the present disclosure provide significant advantages for the treatment of autoimmune diseases.

Exemplary autoimmune diseases that can be treated using the compositions and methods of the present disclosure include type 1 diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barré, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Ménière's Disease, Mixed Connective Tissue Disease, Multiple Sclerosis, Myasthenia Gravis, Neuromyelitis Optica, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis, among others.

### Methods of Treating Multiple Sclerosis

Multiple sclerosis (MS) is an autoimmune demyelinating disease in which the insulating covers of nerve cells in the brain and spinal cord are damaged. This damage disrupts the ability of parts of the nervous system to communicate and results in persistent neurological damage. Patients with MS can exhibit a wide range of symptoms including, for example, numbness or tingling, weakness, dizziness, tremor, lack of coordination, unsteady gait, vision problems, pain, and fatigue.

Using the compositions and methods of the disclosure, a patient, such as a human patient suffering from MS, may be administered a population of pluripotent cells, such as pluripotent hematopoietic cells (e.g., HSCs), that include a nucleic acid cassette that encodes a protein (e.g., a chimeric antigen receptor) that binds myelin oligodendrocyte glycoprotein and that is operably linked to one or more lineage-specific transcription regulatory elements that are active in CD4+CD25+ Treg cells. The pluripotent hematopoietic cells may ameliorate one or more symptoms of the disease, slow or halt progression of the disease, and/or treat one or more underlying physiological causes of the disease.

### Methods of Treating Type 1 Diabetes

Diabetes is a severe autoimmune disease that is characterized by insulin deficiency that prevents normal regulation of blood glucose levels. Insulin is a peptide hormone produced by p cells within the islets of Langerhans of the pancreas (P-islet cells). Insulin promotes glucose utilization, protein synthesis, formation and storage of neutral lipids, and is the primary source of energy for brain and muscle tissue. Type 1 diabetes is caused by an autoimmune reaction that results in destruction of the p-islet cells of the pancreas, which eliminates or reduces insulin production and eventually results in hyperglycemia and ketoacidosis. Examples of symptoms of Type 1 diabetes include increased thirst, frequent urination, extreme hunger, weight loss, fatigue, and blurred vision. The chronic hyperglycemia of type 1 diabetes is also associated with significant and often devastating long-term complications in the eyes, kidneys, nerves, and blood vessels.

Using the compositions and methods of the disclosure, a patient, such as a human patient suffering from type 1 diabetes, may be administered a population of pluripotent cells, such as pluripotent hematopoietic cells (e.g., HSCs), that include a nucleic acid cassette that encodes a protein (e.g., a chimeric antigen receptor) that binds insulin, GAD-65, IA-2, or ZnT8, and that is operably linked to one or more lineage-specific transcription regulatory elements that are active in CD4+CD25+ Treg cells. The pluripotent hematopoietic cells may ameliorate one or more symptoms of the disease, slow or halt progression of the disease, and/or treat one or more underlying physiological causes of the disease.

### Methods of Treating Rheumatoid Arthritis

Rheumatoid arthritis is an autoimmune disease in which the synovial membranes lining the joints become inflamed. Over time, the inflammation may destroy the joint tissues, leading to disability. Examples of symptoms of rheumatoid arthritis include inflammation, fatigue, weakness, and painful, swollen, and/or tender joints.

Using the compositions and methods of the disclosure, a patient, such as a human patient suffering from rheumatoid arthritis, may be administered a population of pluripotent cells, such as pluripotent hematopoietic cells (e.g., HSCs), that include a nucleic acid cassette that encodes a protein (e.g., a chimeric antigen receptor) that binds collagen II, the Fc portion of immunoglobin, citrullinated peptides, carbamylated peptides, or HSP65, and that is operably linked to one or more lineage-specific transcription regulatory elements that are active in CD4+CD25+ Treg cells. The pluripotent hematopoietic cells may ameliorate one or more symptoms of the disease, slow or halt progression of the disease, and/or treat one or more underlying physiological causes of the disease.

### Cells for Lineage-Specific Expression of an Autoantigen-Binding Protein

Cells that may be used in conjunction with the compositions and methods described herein include cells that are capable of undergoing further differentiation. For example, one type of cell that can be used in conjunction with the compositions and methods described herein is a pluripotent cell, which possesses the ability to develop into more than one differentiated cell type. An example of a pluripotent cell includes a pluripotent hematopoietic cell, which has the ability to develop into more than one differentiated cell type of the hematopoietic lineage. Pluripotent hematopoietic cells that may be used in conjunction with the compositions and methods described herein include, for example, HSCs, HPCs, ESCs, iPSCs, lymphoid progenitor cells, and CD34+ cells. HSCs are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells).

One advantage of using pluripotent hematopoietic cells (e.g., HSCs) in conjunction with the compositions and methods described herein is that these cells have the ability to differentiate into Treg cells. While pluripotent hematopoietic cells can also differentiate into blood cells of lineages that are distinct from Treg cells, using the compositions and methods described herein, an autoantigen-binding protein may be preferentially expressed in cells that differentiate into Treg cells. The compositions and methods of the disclosure may achieve excellent specificity of the autoantigen-binding protein in Treg cells by controlling expression of the autoantigen-binding protein with lineage-specific regulatory elements that are preferentially active in CD4+CD25+ Treg cells.

### Lineage-Specific Transcription Regulatory Elements

Expression of the Foxp3 transcription factor is a distinctive feature of Treg cells and is responsible for much of the immunosuppressive phenotype displayed by these cells. Regulation of Foxp3 expression by transcription regulatory elements (e.g., a Foxp3 promoter, a CNS1 enhancer, a CNS2 enhancer, a CNS3 enhancer, and/or a CNS0 enhancer) is important to maintain homeostasis of Treg-cell-meditated immune responses. The compositions and methods of the disclosure utilize Treg-specific transcription regulatory elements, such as Foxp3 transcription regulatory elements, to drive the expression of a nucleic acid cassette encoding an autoantigen binding-protein, as described herein, specifically in Treg cells.

Transcription regulatory elements that may be used in conjunction with the compositions and methods described herein may contain various portions operably linked to one another. For example, transcription regulatory elements described herein may contain a Foxp3 promoter, or a functional portion thereof. The Foxp3 promoter turns on transcription of the Foxp3 gene in Treg cells. Transcription factors may bind to a Foxp3 promoter region, as described herein, and transactivate the Foxp3 gene. Examples of transcription factors that bind to a Foxp3 promoter region include Foxo transcription factor family members (e.g., Foxo1 and Foxo3) and Nr4a nuclear receptor family members (e.g., Nr4a1 (Nur77), Nr4a2, and Nr4a3), as described in Lee et al., Exp. Mol. Med. 50(3):e456 (2018). An exemplary regulatory element containing a human Foxp3 promoter region contains, for example, from nucleic acids −511 to +176, with respect to the Foxp3 transcription start site of the human Foxp3 locus, as set forth in SEQ ID NO: 1 and as described in Mantel et al., J. Immunol. 176(6):3593-602 (2006). Another example of a regulatory element containing a human Foxp3 promoter region is set forth in SEQ ID NO: 2, as described in Kim et al., J. Exp. Med. 204(7):1543-51 (2007). An exemplary regulatory element containing a murine Foxp3 promoter region is set forth, for example, in SEQ ID NO: 3, as described in Zheng et al., Nature. 463(7282):808-12 (2010). By way of alignment of SEQ ID NO: 3 to the human genome, a further example of a regulatory element containing a human Foxp3 promoter region is set forth in SEQ ID NO: 4. Additional nucleic acid regulatory elements useful in conjunction with the compositions and methods described herein include nucleic acid molecules that have at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

The mechanism underlying Treg-specific expression of Foxp3 may involve other cis-regulatory elements, such as conserved non-coding sequences (CNSs).

Additionally or alternatively, transcription regulatory elements described herein may contain a CNS1 enhancer, or a functional portion thereof. CNS1 contains the transforming growth factor-P (TGF-p) response element, which contributes to peripheral induction of Treg cells and mucosal immune tolerance. Deletion of CNS1 has been shown to markedly reduce the Treg cell population in gut-associated lymphoid tissue. Transcription factors may bind to a CNS1 enhancer region, as described herein, and transactivate the Foxp3 gene. Examples of transcription factors that bind to a CNS1 enhancer region include AP-1, NFAT, Smad3, and Foxo (e.g., Foxo1 and Foxo3) transcription factors, as described in Lee et al., Exp. Mol. Med. 50(3):e456 (2018). An exemplary regulatory element containing a human CNS1 enhancer region contains, for example, from nucleic acids −500 to +100, with respect to the Foxp3 transcription start site of the human Foxp3 locus, as described in Kim et al., J. Exp. Med. 204(7):1543-51 (2007). An exemplary regulatory element containing a murine CNS1 enhancer region is set forth, for example, in SEQ ID NO: 5, as described in Tone et al., Nat. Immunol. 9(2):194-202 (2008). By way of alignment of SEQ ID NO: 5 to the human genome, an additional example of a regulatory element containing a human CNS1 enhancer region is set forth in SEQ ID NO: 6. Another example of a regulatory element containing a murine CNS1 enhancer region is set forth in SEQ ID NO: 7, as described in Zheng et al., Nature. 463(7282):808-12 (2010). By way of alignment of SEQ ID NO: 7 to the human genome, a further example of a regulatory element containing a human CNS1 enhancer region is set forth in SEQ ID NO: 8. Additional nucleic acid regulatory elements useful in conjunction with the compositions and methods described herein include nucleic acid molecules that have at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

Additionally or alternatively, transcription regulatory elements described herein may contain a CNS2 enhancer, or a functional portion thereof. CNS2 contains CpG islands that are highly demethylated only in functional Treg cells. Demethylation of CNS2 is considered to be the most definitive marker of commitment to the Treg cell lineage. CNS2 is responsible for the stability of Foxp3 expression in response to T cell receptor stimulation and during Treg cell proliferation. Transcription factors may bind to a CNS2 enhancer region, as described herein, and transactivate the Foxp3 gene. Examples of transcription factors that bind to a CNS2 enhancer region include Runx, Foxp3, Ets-1, CREB, Stat5, NFAT, and c-Rel, as described in Lee et al., Exp. Mol. Med. 50(3):e456 (2018). An exemplary regulatory element containing a human CNS2 enhancer region contains, for example, from nucleic acids +2,022 to +2,721, with respect to the Foxp3 transcription start site of the human Foxp3 locus, as described in Kim et al., J. Exp. Med. 204(7):1543-51 (2007). An exemplary regulatory element containing a murine CNS2 enhancer region is set forth, for example, in SEQ ID NO: 9, as described in Kawakami et al., Immunity. 54(5):947-961 (2021). By way of alignment of SEQ ID NO: 9 to the human genome, an additional example of a regulatory element containing a human CNS2 enhancer region is set forth in SEQ ID NO: 10. Another example of a regulatory element containing a murine CNS2 enhancer region is set forth in SEQ ID NO: 11, as described in Zheng et al., Nature. 463(7282):808-12 (2010). By way of alignment of SEQ ID NO: 11 to the human genome, a further example of a regulatory element containing a human CNS2 enhancer region is set forth in SEQ ID NO: 12. Additional nucleic acid regulatory elements useful in conjunction with the compositions and methods described herein include nucleic acid molecules that have at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

Additionally or alternatively, transcription regulatory elements described herein may contain a CNS3 enhancer region, or a functional portion thereof. CNS3 plays a role in thresholding TCR stimuli required for Foxp3 expression and is important for peripheral and thymic Treg cell generation. Transcription factors may bind to a CNS3 enhancer region, as described herein, and transactivate the Foxp3 gene. Examples of transcription factors that bind to a CNS3 enhancer region include Foxo (e.g., Foxo1 and Foxo3) and c-Rel, as described in Lee et al., Exp. Mol. Med. 50(3):e456 (2018). An exemplary regulatory element containing a human CNS3 enhancer region contains, for example, from nucleic acids +4,301 to +4,500, with respect to the Foxp3 transcription start site of the human Foxp3 locus, as described in Kim et al., J. Exp. Med. 204(7):1543-51 (2007). An exemplary regulatory element containing a murine CNS3 enhancer region is set forth, for example, in SEQ ID NO: 13, as described in Kawakami et al., Immunity. 54(5):947-961 (2021). By way of alignment of SEQ ID NO: 13 to the human genome, an additional example of a regulatory element containing a human CNS3 enhancer region is set forth in SEQ ID NO: 14. Another example of a regulatory element containing a murine CNS3 enhancer region is set forth in SEQ ID NO: 15, as described in Zheng et al., Nature. 463(7282):808-12 (2010). By way of alignment of SEQ ID NO: 15 to the human genome, a further example of a regulatory element containing a human CNS3 enhancer region is set forth in SEQ ID NO: 16. Additional nucleic acid regulatory elements useful in conjunction with the compositions and methods described herein include nucleic acid molecules that have at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

Additionally or alternatively, transcription regulatory elements described herein may contain a CNS0 enhancer, or a functional portion thereof. CNS0 is a Treg-cell specific enhancer. Transcription factors may bind to a CNS0 enhancer region and transactivate the Foxp3 gene. Examples of transcription factors that bind to a CNS0 enhancer region include Satb1 and Stat5, as described in Lee et al., Exp. Mol. Med. 50(3):e456 (2018) and Kawakami et al., Immunity. 54(5):947-961 (2021). Satb1, a chromatin organizer, was found to bind CNS0 and act as a pioneer factor to activate Treg cell-specific enhancers of the Foxp3 gene and other Treg cell-related genes such as Ctla4 and Il2ra at the early stages of thymic Treg cell differentiation. Satb1 allows other transcription factors to bind to regulatory elements by binding to closed chromatin and modifying the epigenetic status of the Foxp3 locus to a poised state. An exemplary regulatory element containing a murine CNS0 enhancer region is set forth, for example, in SEQ ID NO: 17, as described in Kawakami et al., Immunity. 54(5):947-961 (2021). By way of alignment of SEQ ID NO: 17 to the human genome, an exemplary regulatory element containing a human CNS0 enhancer region is set forth, for example, in SEQ ID NO: 18. Another example of a regulatory element containing a murine CNS0 enhancer region is set forth in SEQ ID NO: 19, as described in Dikiy et al., Immunity. 54(5):931-946 (2021). By way of alignment of SEQ ID NO: 19 to the human genome, a further example of a regulatory element containing a human CNS0 enhancer region is set forth in SEQ ID NO: 20. Additional nucleic acid regulatory elements useful in conjunction with the compositions and methods described herein include nucleic acid molecules that have at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or greater, sequence identity) with respect to the above nucleic acid sequences.

### Additional Methods of Transcriptional Regulation

Additional transcription regulatory elements may be used in conjunction with the compositions and methods of the disclosure to modulate expression of a nucleic acid cassette encoding an autoantigen-binding protein, as described herein. For example, nucleic acid cassette expression may be controlled at the transcriptional level by operably linked regulatory sequence elements, such as DNA binding domains, that promote or prevent expression of the nucleic acid cassette upon binding of a chimeric transcription factor, containing a DNA-binding domain and a drug-binding domain, in the presence of small molecule activators or drug induction agents. Examples of drug-inducible systems are described in Tristán-Manzano et al., Front. Immunol. 11:2044 (2020), which is incorporated herein by reference.

Engineered riboswitches may also be used in conjunction with the compositions and methods of the disclosure to control transcription of nucleic acid cassettes described herein. These regulatory elements can bind metabolites or metal ions as ligands and regulate mRNA expression by forming alternative structures in response to ligand binding. Using the compositions and methods of the disclosure, exogenous agents, such as ligands, may induce transcription of a nucleic acid cassette that is operably linked to a riboswitch. Exemplary riboswitches are described in Strobel et al. ACS Synth. Biol. 9(6):1292-1305 (2020), which is incorporated herein by reference. Examples of inductor ligands include tetracycline, tetracycline derivatives, rapamycin, theophylline, and guanine. Additional examples of inductor ligands are described in Tickner et al. Pharmaceuticals. 14(6):554 (2021), which is incorporated herein by reference.

Suicide gene safety switches may be used in conjunction with the compositions and methods of the disclosure to control persistence and survival of genetically modified cells, such as pluripotent hematopoietic cells that include a nucleic acid cassette as described herein. For example, nucleic acid cassettes may be operatively linked to suicide gene safety switches, such as the inducible Caspase 9 system (iCasp9) or herpes-simplex-thymidine-kinase (HSV-TK), for selective clearance of transduced genetically modified cells (e.g., pluripotent hematopoietic cells transduced with a lentiviral vector that include a nucleic acid cassette). The induction of iCasp9 depends on the administration of small molecules, such as the dimerizer drug AP1903. Dimerization results in rapid induction of apoptosis in transduced cells, and chimeric proteins composed of a drug binding domain linked in frame with components of the apoptotic pathway can allow for conditional dimerization and apoptosis of the transduced cells after administration of a non-therapeutic small molecule dimerizer. Nucleoside analogues, such as ganciclovir, in combination with HSV-TK can also be used to induce apoptosis. Exemplary suicide gene safety switches are described in Jones et al. Front. Pharmacol. 5:254 (2014), the disclosure of which is incorporated by reference.

Additionally, inhibitory RNA (RNAi) sequences may be used in conjunction with the compositions and methods of the disclosure to regulate transcription of a nucleic acid cassette in Treg cells. For example, RNAi may be used to target microRNAs, such as microRNA-17 (miR-17). miR-17 has been shown to diminish Treg cell suppressive activity by targeting Foxp3 co-regulators, such as Eos, as described in Yang et al. Immunity. 45(1):83-93. (2016), which is incorporated herein by reference. Targeting miR-17 would optimize the suppressive function of genetically modified Treg cells, and limit potential pro-inflammatory or pathogenic cellular activity.

### Autoantigen-Binding Proteins

Treg cells derived from pluripotent cells (e.g., pluripotent hematopoietic cells), as described herein, may express autoantigen-binding proteins that allow the cells to bind to tissue-specific autoantigens and to traffic to sites of autoimmunity, specifically focusing Treg suppressor functions at diseased sites. Examples of autoantigen-binding proteins useful in conjunction with the compositions and methods of the disclosure include single-chain proteins (e.g., chimeric antigen receptors and single-chain antibody fragments) and multi-chain proteins (e.g., T cell receptors, full-length antibodies, dual-variable immunoglobulin domains, diabodies, triabodies, antibody-like protein scaffolds, Fab fragments, and F(ab′)₂ molecules) that specifically bind an antigen that is expressed endogenously in a subject.

Autoantigen-binding proteins, as described herein, may bind to autoantigens such as myelin oligodendrocyte glycoprotein, aquaporin 4, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double-stranded DNA, single-stranded DNA, transferrin, fetuin, factor II, factor VII, fibrin, fibrinogen, C1, C1q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, and proteins expressed in the thyroid gland. Additional examples of autoantigens are described in Quintana et al., J. Autoimmun. 17(3):191-7 (2001) and Riedhammer et al., Front Immunol. 6:322 (2015), the disclosures of which are incorporated herein by reference in their entirety.

### Antibodies

Antibodies that may be used in conjunction with the compositions and methods of the disclosure include any protein or peptide-containing molecule that includes at least a portion of an immunoglobulin molecule, such as, but not limited, to at least one complementarity determining region (CDR) of a heavy or light chain or a ligand-binding portion thereof, a heavy chain or light chain variable region, a heavy chain or light chain constant region, or any portion thereof, that is capable of specifically binding to an antigen that is expressed endogenously in a subject (e.g., a human subject). For instance, two or more portions of an immunoglobulin molecule may be covalently bound to one another, e.g., via an amide bond, a thioether bond, a carbon-carbon bond, a disulfide bridge, or by a linker, such as a linker described herein or known in the art.

Exemplary antibodies that may be used in conjunction with the compositions and methods of the disclosure include polyclonal, monoclonal, genetically engineered, and otherwise modified forms of antibodies, such as chimeric antibodies, human antibodies, humanized antibodies, primatized antibodies, and heteroconjugate antibodies (e.g., bi- tri- and quad-specific antibodies, diabodies, triabodies, and tetrabodies), and antigen-binding fragments of antibodies.

Chimeric antibodies that may be used in conjunction with the compositions and methods described herein may have variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, or bison, among others). Methods for producing chimeric antibodies are known in the art. See, e.g., Morrison, Science. 229(4719):1202-7 (1985); Oi et al. BioTechniques. 4:214-221 (1986); Gillies et al. J. Immunol. Methods. 125:191-202 (1985); U.S. Pat. Nos. 5,807,715; 4,816,567; and 4,816,397.

Human antibodies that may be used in conjunction with the compositions and methods described herein include antibodies in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can include a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598.

Humanized antibodies that may be used in conjunction with the compositions and methods described herein include forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will include substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also include at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

Exemplary antigen-binding fragments of antibodies that may be used in conjunction with the compositions and methods of the disclosure include, for example, a Fab′, F(ab′)₂, Fab, Fv, rIgG, scFv, SMIP, diabody, a triabody, an affibody, a nanobody, an aptamer, or a domain antibody. These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

Single-chain Fv (scFv) molecules that may be used in conjunction with the compositions and methods described herein include antibodies in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of an scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of an scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

Antibody-like protein scaffolds may also be used in conjunction with the compositions and methods of the disclosure, such as the tenth fibronectin type III domain (¹⁰Fn3), which contains BC, DE, and FG structural loops similar in structure and solvent accessibility to antibody CDRs.

### Chimeric Antigen Receptors

CAR Treg cells can be produced by engineering a precursor cell, such as a pluripotent cell (e.g., a pluripotent hematopoietic cell). The engineered pluripotent hematopoietic cells, as described herein, can differentiate into cells that express a CAR that is specific for a target antigen, such as an autoantigen, if the differentiated cell is a Treg cell. The control of CAR expression by lineage-specific transcription regulatory elements that are active in CD4+CD25+ Treg cells (e.g., a Foxp3 promoter) allows for Treg-specific expression of CARs.

Structurally, CARs may contain an extracellular antigen recognition domain, a hinge domain, a transmembrane domain, and an intracellular signaling domain. The antigen recognition domain may contain an antibody or antibody fragment thereof that confers specificity for a target cell by recognizing, and specifically binding to, a given antigen (e.g., an autoantigen). Examples of antigen recognition domains that may be used in conjunction with the methods described herein include single domain antibody fragments (sdAb), single chain antibodies (e.g., an scFv), and humanized antibodies. The hinge domain positions the antigen recognition domain away from the T cell surface to enable proper cell/cell contact, antigen binding, and activation. Exemplary hinge domains for use in conjunction with the methods described herein include those derived from CD8 (e.g., CD8α), CD28, IgG1/IgG4 (hinge-Fc portion), CD4, CD7, and IgD. The transmembrane domain fuses the extracellular antigen recognition domain and the intracellular signaling domain and anchors the CAR to the plasma membrane of the T cell. Exemplary transmembrane domains for use in conjunction with the methods described herein include those derived from CD3 alpha, CD3 beta, CD3 epsilon, CD3 zeta, CD4, CD5, CD8 (e.g., CD8α), CD9, CD16, CD22, CD27, CD28, CD33, CD37, CD45, CD64, CD80, CD86, CD134, CD137, CD152, CD154, PD-1, CD4, FcRIγ, CD7, OX40, and MHC (H2-Kb). The intracellular signaling domain may generate a signal that promotes an immunosuppressive function of the CAR-containing Treg cell and contain a primary intracellular signaling domain and optionally one or more costimulatory intracellular signaling domains. Exemplary primary intracellular signaling domains include those derived from the molecules responsible for primary stimulation, or antigen dependent simulation. For example, a primary intracellular signaling domain may be derived from CD3 zeta, FcR gamma, FcR beta, CD3 gamma, CD3 delta, CD3 epsilon, CD5, CD22, CD79a, CD79b, CD278 (ICOS), CD66d, DAP10, and DAP12. Exemplary costimulatory intracellular signaling domains include those derived from molecules responsible for costimulatory signals, or antigen independent stimulation. For example, a costimulatory intracellular signaling domain may be derived from CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, CD83, CDS, ICAM-1, LFA-1 (CD11a/CD18), an MHC class I molecule, BTLA, or a Toll ligand receptor.

Pluripotent hematopoietic cells can be genetically modified to express an antigen receptor on Treg cells that specifically binds to a particular autoantigen by any of a variety of genome editing techniques described herein or known in the art. Exemplary techniques for modifying a pluripotent hematopoietic cell genome so as to incorporate a gene encoding a chimeric antigen receptor include the CRISPR/Cas, zinc finger nuclease, TALEN, and ARCUS™ platforms.

### Methods of Viral Transduction

### Transduction Using a Poloxamer

Poloxamers may be used in conjunction with the compositions and methods of the disclosure to enhance transduction efficiency. Poloxamers that may be used include those having an average molar mass of polyoxypropylene subunits of greater than 2,050 g/mol (e.g., an average molar mass of polyoxypropylene subunits of about 2,055 g/mol, 2,060 g/mol, 2,075 g/mol, 2,080 g/mol, 2,085 g/mol, 2, 090 g/mol, 2,095 g/mol, 2,100 g/mol, 2,200 g/mol, 2,300 g/mol, 2,400 g/mol, 2,500 g/mol, 2,600 g/mol, 2,700 g/mol, 2,800 g/mol, 2,900 g/mol, 3,000 g/mol, 3,100 g/mol, 3,200 g/mol, 3,300 g/mol, 3,400 g/mol, 3,500 g/mol, 3,600 g/mol, 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of greater than 2,250 g/mol (e.g., an average molar mass of polyoxypropylene subunits of about 2,300 g/mol, 2,400 g/mol, 2,500 g/mol, 2,600 g/mol, 2,700 g/mol, 2,800 g/mol, 2,900 g/mol, 3,000 g/mol, 3,100 g/mol, 3,200 g/mol, 3,300 g/mol, 3,400 g/mol, 3,500 g/mol, 3,600 g/mol, 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of greater than 2,750 g/mol (e.g., an average molar mass of polyoxypropylene subunits of about 2,800 g/mol, 2,900 g/mol, 3,000 g/mol, 3,100 g/mol, 3,200 g/mol, 3,300 g/mol, 3,400 g/mol, 3,500 g/mol, 3,600 g/mol, 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of greater than 3,250 g/mol (e.g., an average molar mass of polyoxypropylene subunits of about 3,300 g/mol, 3,400 g/mol, 3,500 g/mol, 3,600 g/mol, 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of greater than 3,625 g/mol (e.g., an average molar mass of polyoxypropylene subunits of about 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of from about 2,050 g/mol to about 4,000 g/mol (e.g., about 2,050 g/mol, 2,055 g/mol, 2,060 g/mol, 2,065 g/mol, 2,070 g/mol, 2,075 g/mol, 2,080 g/mol, 2,085 g/mol, 2,090 g/mol, 2,095 g/mol, 2,100 g/mol, 2,105 g/mol, 2,110 g/mol, 2,115 g/mol, 2,120 g/mol, 2,125 g/mol, 2,130 g/mol, 2,135 g/mol, 2,140 g/mol, 2,145 g/mol, 2,150 g/mol, 2,155 g/mol, 2,160 g/mol, 2,165 g/mol, 2,170 g/mol, 2,175 g/mol, 2,180 g/mol, 2,185 g/mol, 2,190 g/mol, 2,195 g/mol, 2,200 g/mol, 2,205 g/mol, 2,210 g/mol, 2,215 g/mol, 2,220 g/mol, 2,225 g/mol, 2,230 g/mol, 2,235 g/mol, 2,240 g/mol, 2,245 g/mol, 2,250 g/mol, 2,255 g/mol, 2,260 g/mol, 2,265 g/mol, 2,270 g/mol, 2,275 g/mol, 2,280 g/mol, 2,285 g/mol, 2,290 g/mol, 2,295 g/mol, 2,300 g/mol, 2,305 g/mol, 2,310 g/mol, 2,315 g/mol, 2,320 g/mol, 2,325 g/mol, 2,330 g/mol, 2,335 g/mol, 2,340 g/mol, 2,345 g/mol, 2,350 g/mol, 2,355 g/mol, 2,360 g/mol, 2,365 g/mol, 2,370 g/mol, 2,375 g/mol, 2,380 g/mol, 2,385 g/mol, 2,390 g/mol, 2,395 g/mol, 2,400 g/mol, 2,405 g/mol, 2,410 g/mol, 2,415 g/mol, 2,420 g/mol, 2,425 g/mol, 2,430 g/mol, 2,435 g/mol, 2,440 g/mol, 2,445 g/mol, 2,450 g/mol, 2,455 g/mol, 2,460 g/mol, 2,465 g/mol, 2,470 g/mol, 2,475 g/mol, 2,480 g/mol, 2,485 g/mol, 2,490 g/mol, 2,495 g/mol, 2,500 g/mol, 2,505 g/mol, 2,510 g/mol, 2,515 g/mol, 2,520 g/mol, 2,525 g/mol, 2,530 g/mol, 2,535 g/mol, 2,540 g/mol, 2,545 g/mol, 2,550 g/mol, 2,555 g/mol, 2,560 g/mol, 2,565 g/mol, 2,570 g/mol, 2,575 g/mol, 2,580 g/mol, 2,585 g/mol, 2,590 g/mol, 2,595 g/mol, 2,600 g/mol, 2,605 g/mol, 2,610 g/mol, 2,615 g/mol, 2,620 g/mol, 2,625 g/mol, 2,630 g/mol, 2,635 g/mol, 2,640 g/mol, 2,645 g/mol, 2,650 g/mol, 2,655 g/mol, 2,660 g/mol, 2,665 g/mol, 2,670 g/mol, 2,675 g/mol, 2,680 g/mol, 2,685 g/mol, 2,690 g/mol, 2,695 g/mol, 2,700 g/mol, 2,705 g/mol, 2,710 g/mol, 2,715 g/mol, 2,720 g/mol, 2,725 g/mol, 2,730 g/mol, 2,735 g/mol, 2,740 g/mol, 2,745 g/mol, 2,750 g/mol, 2,755 g/mol, 2,760 g/mol, 2,765 g/mol, 2,770 g/mol, 2,775 g/mol, 2,780 g/mol, 2,785 g/mol, 2,790 g/mol, 2,795 g/mol, 2,800 g/mol, 2,805 g/mol, 2,810 g/mol, 2,815 g/mol, 2,820 g/mol, 2,825 g/mol, 2,830 g/mol, 2,835 g/mol, 2,840 g/mol, 2,845 g/mol, 2,850 g/mol, 2,855 g/mol, 2,860 g/mol, 2,865 g/mol, 2,870 g/mol, 2,875 g/mol, 2,880 g/mol, 2,885 g/mol, 2,890 g/mol, 2,895 g/mol, 2,900 g/mol, 2,905 g/mol, 2,910 g/mol, 2,915 g/mol, 2,920 g/mol, 2,925 g/mol, 2,930 g/mol, 2,935 g/mol, 2,940 g/mol, 2,945 g/mol, 2,950 g/mol, 2,955 g/mol, 2,960 g/mol, 2,965 g/mol, 2,970 g/mol, 2,975 g/mol, 2,980 g/mol, 2,985 g/mol, 2,990 g/mol, 2,995 g/mol, 3,000 g/mol, 3,005 g/mol, 3,010 g/mol, 3,015 g/mol, 3,020 g/mol, 3,025 g/mol, 3,030 g/mol, 3,035 g/mol, 3,040 g/mol, 3,045 g/mol, 3,050 g/mol, 3,055 g/mol, 3,060 g/mol, 3,065 g/mol, 3,070 g/mol, 3,075 g/mol, 3,080 g/mol, 3,085 g/mol, 3,090 g/mol, 3,095 g/mol, 3,100 g/mol, 3,105 g/mol, 3,110 g/mol, 3,115 g/mol, 3,120 g/mol, 3,125 g/mol, 3,130 g/mol, 3,135 g/mol, 3,140 g/mol, 3,145 g/mol, 3,150 g/mol, 3,155 g/mol, 3,160 g/mol, 3,165 g/mol, 3,170 g/mol, 3,175 g/mol, 3,180 g/mol, 3,185 g/mol, 3,190 g/mol, 3,195 g/mol, 3,200 g/mol, 3,205 g/mol, 3,210 g/mol, 3,215 g/mol, 3,220 g/mol, 3,225 g/mol, 3,230 g/mol, 3,235 g/mol, 3,240 g/mol, 3,245 g/mol, 3,250 g/mol, 3,255 g/mol, 3,260 g/mol, 3,265 g/mol, 3,270 g/mol, 3,275 g/mol, 3,280 g/mol, 3,285 g/mol, 3,290 g/mol, 3,295 g/mol, 3,300 g/mol, 3,305 g/mol, 3,310 g/mol, 3,315 g/mol, 3,320 g/mol, 3,325 g/mol, 3,330 g/mol, 3,335 g/mol, 3,340 g/mol, 3,345 g/mol, 3,350 g/mol, 3,355 g/mol, 3,360 g/mol, 3,365 g/mol, 3,370 g/mol, 3,375 g/mol, 3,380 g/mol, 3,385 g/mol, 3,390 g/mol, 3,395 g/mol, 3,400 g/mol, 3,405 g/mol, 3,410 g/mol, 3,415 g/mol, 3,420 g/mol, 3,425 g/mol, 3,430 g/mol, 3,435 g/mol, 3,440 g/mol, 3,445 g/mol, 3,450 g/mol, 3,455 g/mol, 3,460 g/mol, 3,465 g/mol, 3,470 g/mol, 3,475 g/mol, 3,480 g/mol, 3,485 g/mol, 3,490 g/mol, 3,495 g/mol, 3,500 g/mol, 3,505 g/mol, 3,510 g/mol, 3,515 g/mol, 3,520 g/mol, 3,525 g/mol, 3,530 g/mol, 3,535 g/mol, 3,540 g/mol, 3,545 g/mol, 3,550 g/mol, 3,555 g/mol, 3,560 g/mol, 3,565 g/mol, 3,570 g/mol, 3,575 g/mol, 3,580 g/mol, 3,585 g/mol, 3,590 g/mol, 3,595 g/mol, 3,600 g/mol, 3,605 g/mol, 3,610 g/mol, 3,615 g/mol, 3,620 g/mol, 3,625 g/mol, 3,630 g/mol, 3,635 g/mol, 3,640 g/mol, 3,645 g/mol, 3,650 g/mol, 3,655 g/mol, 3,660 g/mol, 3,665 g/mol, 3,670 g/mol, 3,675 g/mol, 3,680 g/mol, 3,685 g/mol, 3,690 g/mol, 3,695 g/mol, 3,700 g/mol, 3,705 g/mol, 3,710 g/mol, 3,715 g/mol, 3,720 g/mol, 3,725 g/mol, 3,730 g/mol, 3,735 g/mol, 3,740 g/mol, 3,745 g/mol, 3,750 g/mol, 3,755 g/mol, 3,760 g/mol, 3,765 g/mol, 3,770 g/mol, 3,775 g/mol, 3,780 g/mol, 3,785 g/mol, 3,790 g/mol, 3,795 g/mol, 3,800 g/mol, 3,805 g/mol, 3,810 g/mol, 3,815 g/mol, 3,820 g/mol, 3,825 g/mol, 3,830 g/mol, 3,835 g/mol, 3,840 g/mol, 3,845 g/mol, 3,850 g/mol, 3,855 g/mol, 3,860 g/mol, 3,865 g/mol, 3,870 g/mol, 3,875 g/mol, 3,880 g/mol, 3,885 g/mol, 3,890 g/mol, 3,895 g/mol, 3,900 g/mol, 3,905 g/mol, 3,910 g/mol, 3,915 g/mol, 3,920 g/mol, 3,925 g/mol, 3,930 g/mol, 3,935 g/mol, 3,940 g/mol, 3,945 g/mol, 3,950 g/mol, 3,955 g/mol, 3,960 g/mol, 3,965 g/mol, 3,970 g/mol, 3,975 g/mol, 3,980 g/mol, 3,985 g/mol, 3,990 g/mol, 3,995 g/mol, or 4,000 g/mol).

In some embodiments, the poloxamer has an average molar mass of polyoxypropylene subunits of from about 2,750 g/mol to about 4,000 g/mol (e.g., about 2,750 g/mol, 2,755 g/mol, 2,760 g/mol, 2,765 g/mol, 2,770 g/mol, 2,775 g/mol, 2,780 g/mol, 2,785 g/mol, 2,790 g/mol, 2,795 g/mol, 2,800 g/mol, 2,805 g/mol, 2,810 g/mol, 2,815 g/mol, 2,820 g/mol, 2,825 g/mol, 2,830 g/mol, 2,835 g/mol, 2,840 g/mol, 2,845 g/mol, 2,850 g/mol, 2,855 g/mol, 2,860 g/mol, 2,865 g/mol, 2,870 g/mol, 2,875 g/mol, 2,880 g/mol, 2,885 g/mol, 2,890 g/mol, 2,895 g/mol, 2,900 g/mol, 2,905 g/mol, 2,910 g/mol, 2,915 g/mol, 2,920 g/mol, 2,925 g/mol, 2,930 g/mol, 2,935 g/mol, 2,940 g/mol, 2,945 g/mol, 2,950 g/mol, 2,955 g/mol, 2,960 g/mol, 2,965 g/mol, 2,970 g/mol, 2,975 g/mol, 2,980 g/mol, 2,985 g/mol, 2,990 g/mol, 2,995 g/mol, 3,000 g/mol, 3,005 g/mol, 3,010 g/mol, 3,015 g/mol, 3,020 g/mol, 3,025 g/mol, 3,030 g/mol, 3,035 g/mol, 3,040 g/mol, 3,045 g/mol, 3,050 g/mol, 3,055 g/mol, 3,060 g/mol, 3,065 g/mol, 3,070 g/mol, 3,075 g/mol, 3,080 g/mol, 3,085 g/mol, 3,090 g/mol, 3,095 g/mol, 3,100 g/mol, 3,105 g/mol, 3,110 g/mol, 3,115 g/mol, 3,120 g/mol, 3,125 g/mol, 3,130 g/mol, 3,135 g/mol, 3,140 g/mol, 3,145 g/mol, 3,150 g/mol, 3,155 g/mol, 3,160 g/mol, 3,165 g/mol, 3,170 g/mol, 3,175 g/mol, 3,180 g/mol, 3,185 g/mol, 3,190 g/mol, 3,195 g/mol, 3,200 g/mol, 3,205 g/mol, 3,210 g/mol, 3,215 g/mol, 3,220 g/mol, 3,225 g/mol, 3,230 g/mol, 3,235 g/mol, 3,240 g/mol, 3,245 g/mol, 3,250 g/mol, 3,255 g/mol, 3,260 g/mol, 3,265 g/mol, 3,270 g/mol, 3,275 g/mol, 3,280 g/mol, 3,285 g/mol, 3,290 g/mol, 3,295 g/mol, 3,300 g/mol, 3,305 g/mol, 3,310 g/mol, 3,315 g/mol, 3,320 g/mol, 3,325 g/mol, 3,330 g/mol, 3,335 g/mol, 3,340 g/mol, 3,345 g/mol, 3,350 g/mol, 3,355 g/mol, 3,360 g/mol, 3,365 g/mol, 3,370 g/mol, 3,375 g/mol, 3,380 g/mol, 3,385 g/mol, 3,390 g/mol, 3,395 g/mol, 3,400 g/mol, 3,405 g/mol, 3,410 g/mol, 3,415 g/mol, 3,420 g/mol, 3,425 g/mol, 3,430 g/mol, 3,435 g/mol, 3,440 g/mol, 3,445 g/mol, 3,450 g/mol, 3,455 g/mol, 3,460 g/mol, 3,465 g/mol, 3,470 g/mol, 3,475 g/mol, 3,480 g/mol, 3,485 g/mol, 3,490 g/mol, 3,495 g/mol, 3,500 g/mol, 3,505 g/mol, 3,510 g/mol, 3,515 g/mol, 3,520 g/mol, 3,525 g/mol, 3,530 g/mol, 3,535 g/mol, 3,540 g/mol, 3,545 g/mol, 3,550 g/mol, 3,555 g/mol, 3,560 g/mol, 3,565 g/mol, 3,570 g/mol, 3,575 g/mol, 3,580 g/mol, 3,585 g/mol, 3,590 g/mol, 3,595 g/mol, 3,600 g/mol, 3,605 g/mol, 3,610 g/mol, 3,615 g/mol, 3,620 g/mol, 3,625 g/mol, 3,630 g/mol, 3,635 g/mol, 3,640 g/mol, 3,645 g/mol, 3,650 g/mol, 3,655 g/mol, 3,660 g/mol, 3,665 g/mol, 3,670 g/mol, 3,675 g/mol, 3,680 g/mol, 3,685 g/mol, 3,690 g/mol, 3,695 g/mol, 3,700 g/mol, 3,705 g/mol, 3,710 g/mol, 3,715 g/mol, 3,720 g/mol, 3,725 g/mol, 3,730 g/mol, 3,735 g/mol, 3,740 g/mol, 3,745 g/mol, 3,750 g/mol, 3,755 g/mol, 3,760 g/mol, 3,765 g/mol, 3,770 g/mol, 3,775 g/mol, 3,780 g/mol, 3,785 g/mol, 3,790 g/mol, 3,795 g/mol, 3,800 g/mol, 3,805 g/mol, 3,810 g/mol, 3,815 g/mol, 3,820 g/mol, 3,825 g/mol, 3,830 g/mol, 3,835 g/mol, 3,840 g/mol, 3,845 g/mol, 3,850 g/mol, 3,855 g/mol, 3,860 g/mol, 3,865 g/mol, 3,870 g/mol, 3,875 g/mol, 3,880 g/mol, 3,885 g/mol, 3,890 g/mol, 3,895 g/mol, 3,900 g/mol, 3,905 g/mol, 3,910 g/mol, 3,915 g/mol, 3,920 g/mol, 3,925 g/mol, 3,930 g/mol, 3,935 g/mol, 3,940 g/mol, 3,945 g/mol, 3,950 g/mol, 3,955 g/mol, 3,960 g/mol, 3,965 g/mol, 3,970 g/mol, 3,975 g/mol, 3,980 g/mol, 3,985 g/mol, 3,990 g/mol, 3,995 g/mol, or 4,000 g/mol).

In some embodiments, the poloxamer has an average molar mass of polyoxypropylene subunits of from about 3,250 g/mol to about 4,000 g/mol (e.g., about 3,250 g/mol, 3,255 g/mol, 3,260 g/mol, 3,265 g/mol, 3,270 g/mol, 3,275 g/mol, 3,280 g/mol, 3,285 g/mol, 3,290 g/mol, 3,295 g/mol, 3,300 g/mol, 3,305 g/mol, 3,310 g/mol, 3,315 g/mol, 3,320 g/mol, 3,325 g/mol, 3,330 g/mol, 3,335 g/mol, 3,340 g/mol, 3,345 g/mol, 3,350 g/mol, 3,355 g/mol, 3,360 g/mol, 3,365 g/mol, 3,370 g/mol, 3,375 g/mol, 3,380 g/mol, 3,385 g/mol, 3,390 g/mol, 3,395 g/mol, 3,400 g/mol, 3,405 g/mol, 3,410 g/mol, 3,415 g/mol, 3,420 g/mol, 3,425 g/mol, 3,430 g/mol, 3,435 g/mol, 3,440 g/mol, 3,445 g/mol, 3,450 g/mol, 3,455 g/mol, 3,460 g/mol, 3,465 g/mol, 3,470 g/mol, 3,475 g/mol, 3,480 g/mol, 3,485 g/mol, 3,490 g/mol, 3,495 g/mol, 3,500 g/mol, 3,505 g/mol, 3,510 g/mol, 3,515 g/mol, 3,520 g/mol, 3,525 g/mol, 3,530 g/mol, 3,535 g/mol, 3,540 g/mol, 3,545 g/mol, 3,550 g/mol, 3,555 g/mol, 3,560 g/mol, 3,565 g/mol, 3,570 g/mol, 3,575 g/mol, 3,580 g/mol, 3,585 g/mol, 3,590 g/mol, 3,595 g/mol, 3,600 g/mol, 3,605 g/mol, 3,610 g/mol, 3,615 g/mol, 3,620 g/mol, 3,625 g/mol, 3,630 g/mol, 3,635 g/mol, 3,640 g/mol, 3,645 g/mol, 3,650 g/mol, 3,655 g/mol, 3,660 g/mol, 3,665 g/mol, 3,670 g/mol, 3,675 g/mol, 3,680 g/mol, 3,685 g/mol, 3,690 g/mol, 3,695 g/mol, 3,700 g/mol, 3,705 g/mol, 3,710 g/mol, 3,715 g/mol, 3,720 g/mol, 3,725 g/mol, 3,730 g/mol, 3,735 g/mol, 3,740 g/mol, 3,745 g/mol, 3,750 g/mol, 3,755 g/mol, 3,760 g/mol, 3,765 g/mol, 3,770 g/mol, 3,775 g/mol, 3,780 g/mol, 3,785 g/mol, 3,790 g/mol, 3,795 g/mol, 3,800 g/mol, 3,805 g/mol, 3,810 g/mol, 3,815 g/mol, 3,820 g/mol, 3,825 g/mol, 3,830 g/mol, 3,835 g/mol, 3,840 g/mol, 3,845 g/mol, 3,850 g/mol, 3,855 g/mol, 3,860 g/mol, 3,865 g/mol, 3,870 g/mol, 3,875 g/mol, 3,880 g/mol, 3,885 g/mol, 3,890 g/mol, 3,895 g/mol, 3,900 g/mol, 3,905 g/mol, 3,910 g/mol, 3,915 g/mol, 3,920 g/mol, 3,925 g/mol, 3,930 g/mol, 3,935 g/mol, 3,940 g/mol, 3,945 g/mol, 3,950 g/mol, 3,955 g/mol, 3,960 g/mol, 3,965 g/mol, 3,970 g/mol, 3,975 g/mol, 3,980 g/mol, 3,985 g/mol, 3,990 g/mol, 3,995 g/mol, or 4,000 g/mol).

In some embodiments, the poloxamer has an average molar mass of polyoxypropylene subunits of from about 3,625 g/mol to about 4,000 g/mol (e.g., about 3,625 g/mol, 3,630 g/mol, 3,635 g/mol, 3,640 g/mol, 3,645 g/mol, 3,650 g/mol, 3,655 g/mol, 3,660 g/mol, 3,665 g/mol, 3,670 g/mol, 3,675 g/mol, 3,680 g/mol, 3,685 g/mol, 3,690 g/mol, 3,695 g/mol, 3,700 g/mol, 3,705 g/mol, 3,710 g/mol, 3,715 g/mol, 3,720 g/mol, 3,725 g/mol, 3,730 g/mol, 3,735 g/mol, 3,740 g/mol, 3,745 g/mol, 3,750 g/mol, 3,755 g/mol, 3,760 g/mol, 3,765 g/mol, 3,770 g/mol, 3,775 g/mol, 3,780 g/mol, 3,785 g/mol, 3,790 g/mol, 3,795 g/mol, 3,800 g/mol, 3,805 g/mol, 3,810 g/mol, 3,815 g/mol, 3,820 g/mol, 3,825 g/mol, 3,830 g/mol, 3,835 g/mol, 3,840 g/mol, 3,845 g/mol, 3,850 g/mol, 3,855 g/mol, 3,860 g/mol, 3,865 g/mol, 3,870 g/mol, 3,875 g/mol, 3,880 g/mol, 3,885 g/mol, 3,890 g/mol, 3,895 g/mol, 3,900 g/mol, 3,905 g/mol, 3,910 g/mol, 3,915 g/mol, 3,920 g/mol, 3,925 g/mol, 3,930 g/mol, 3,935 g/mol, 3,940 g/mol, 3,945 g/mol, 3,950 g/mol, 3,955 g/mol, 3,960 g/mol, 3,965 g/mol, 3,970 g/mol, 3,975 g/mol, 3,980 g/mol, 3,985 g/mol, 3,990 g/mol, 3,995 g/mol, or 4,000 g/mol).

In some embodiments, the poloxamer has an average ethylene oxide content of greater than 40% by mass (e.g., about 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the poloxamer has an average ethylene oxide content of greater than 50% by mass (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the poloxamer has an average ethylene oxide content of greater than 60% by mass (e.g., about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the poloxamer has an average ethylene oxide content of greater than 70% by mass (e.g., about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the poloxamer has an average ethylene oxide content of from about 40% to about 90% (e.g., about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%).

In some embodiments, the poloxamer has an average ethylene oxide content of from about 50% to about 85% (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, or 85%).

In some embodiments, the poloxamer has an average ethylene oxide content of from about 60% to about 80% (e.g., about 60%, 61%, 62%, 63%, 64%, 65%, 66%, 67%, 68%, 69%, 70%, 71%, 72%, 73%, 74%, 75%, 76%, 77%, 78%, 79%, 80%).

In some embodiments, the poloxamer has an average molar mass of greater than 10,000 g/mol (e.g., about 10,100 g/mol, 10,200 g/mol, 10,300 g/mol, 10,400 g/mol, 10,500 g/mol, 10,600 g/mol, 10,700 g/mol, 10,800 g/mol, 10,900 g/mol, 11,000 g/mol, 11,100 g/mol, 11,200 g/mol, 11,300 g/mol, 11,400 g/mol, 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of greater than 11,000 g/mol (e.g., about 11,100 g/mol, 11,200 g/mol, 11,300 g/mol, 11,400 g/mol, 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of greater than 12,000 g/mol (e.g., about 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of greater than 12,500 g/mol (e.g., about 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 10,000 g/mol to about 15,000 g/mol (e.g., about 10,000 g/mol, 10,100 g/mol, 10,200 g/mol, 10,300 g/mol, 10,400 g/mol, 10,500 g/mol, 10,600 g/mol, 10,700 g/mol, 10,800 g/mol, 10,900 g/mol, 11,000 g/mol, 11,100 g/mol, 11,200 g/mol, 11,300 g/mol, 11,400 g/mol, 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 11,000 g/mol to about 15,000 g/mol (e.g., about 11,000 g/mol, 11,100 g/mol, 11,200 g/mol, 11,300 g/mol, 11,400 g/mol, 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 11,500 g/mol to about 15,000 g/mol (e.g., about 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 12,000 g/mol to about 15,000 g/mol (e.g., about 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 12,500 g/mol to about 15,000 g/mol (e.g., about 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

### Poloxamers P288, P335, P338, and P407

Poloxamers that may be used in conjunction with the compositions and methods of the disclosure include “poloxamer 288” (also referred to in the art as “P 288” and poloxamer “F98”) having the approximate chemical formula HO(C₂H₄O)ₓ(C₃H₆O)_{y}(C₂H₄O)_{z}H, wherein the sum of x and y is about 236.36, and z is about 44.83. The average molecular weight of P288 is about 13,000 g/mol.

In some embodiments, the poloxamer is a variant of P288, such as a variant of the formula HO(C₂H₄O)ₓ(C₃H₆O)_{y}(C₂H₄O)_{z}H, wherein the sum of x and y is from about 220 to about 250, and z is from about 40 to about 50. In some embodiments, the average molecular weight of the poloxamer is from about 12,000 g/mol to about 14,000 g/mol.

Poloxamers that may be used in conjunction with the compositions and methods of the disclosure further include “poloxamer 335” (also referred to in the art as “P 335” and poloxamer “P105”), having the approximate chemical formula HO(C₂H₄O)ₓ(C₃H₆O)_{y}(C₂H₄O)_{z}H, wherein the sum of x and y is about 73.86, and z is about 56.03. The average molecular weight of P335 is about 6,500 g/mol.

In some embodiments, the poloxamer is a variant of P335, such as a variant of the formula HO(C₂H₄O)ₓ(C₃H₆O)_{y}(C₂H₄O)_{z}H, wherein the sum of x and y is from about 60 to about 80, and z is from about 50 to about 60. In some embodiments, the average molecular weight of the poloxamer is from about 6,000 g/mol to about 7,000 g/mol.

Poloxamers that may be used in conjunction with the compositions and methods of the disclosure further include “poloxamer 338” (also referred to in the art as “P 338” and poloxamer “F108”), having the approximate chemical formula HO(C₂H₄O)ₓ(C₃H₆O)_{y}(C₂H₄O)_{z}H, wherein the sum of x and y is about 265.45, and z is about 50.34. The average molecular weight of P335 is about 14,600 g/mol.

In some embodiments, the poloxamer is a variant of P338, such as a variant of the formula HO(C₂H₄O)ₓ(C₃H₆O)_{y}(C₂H₄O)_{z}H, wherein the sum of x and y is from about 260 to about 270, and z is from about 45 to about 55. In some embodiments, the average molecular weight of the poloxamer is from about 14,000 g/mol to about 15,000 g/mol.

Poloxamers that may be used in conjunction with the compositions and methods of the disclosure further include “poloxamer 407” (also referred to in the art as “P 407” and poloxamer “F127”), having the approximate chemical formula HO(C₂H₄O)ₓ(C₃H₆₀)_{y}(C₂H₄O)_{z}H, wherein the sum of x and y is about 200.45, and z is about 65.17. The average molecular weight of P335 is about 12,600 g/mol.

In some embodiments, the poloxamer is a variant of P407, such as a variant of the formula HO(C₂H₄O)ₓ(C₃H₆₀)_{y}(C₂H₄O)_{z}H, wherein the sum of x and y is from about 190 to about 210, and z is from about 60 to about 70. In some embodiments, the average molecular weight of the poloxamer is from about 12,000 g/mol to about 13,000 g/mol.

For clarity, the terms “average molar mass” and “average molecular weight” are used interchangeable herein to refer to the same quantity. The average molar mass, ethylene oxide content, and propylene oxide content of a poloxamer, as described herein, can be determined using methods disclosed in Alexandridis and Hatton, Colloids and Surfaces A: Physicochemical and Engineering Aspects 96:1-46 (1995), the disclosure of which is incorporated herein by reference in its entirety.

### Transduction Using a Protein Kinase C Modulator

A variety of agents can be used to reduce PKC activity and/or expression. Without being limited by mechanism, such agents can augment viral transduction by stimulating Akt signal transduction and/or maintaining cofilin in a dephosphorylated state, thereby promoting actin depolymerization. This actin depolymerization event may serve to remove a physical barrier that hinders entry of a viral vector into the nucleus of a target cell.

### Staurosporine and Variants Thereof

In some embodiments, the substance that reduces activity and/or expression of PKC is a PKC inhibitor. The PKC inhibitor may be staurosporine or a variant thereof. For example, the PKC inhibitor may be a compound represented by formula (1)

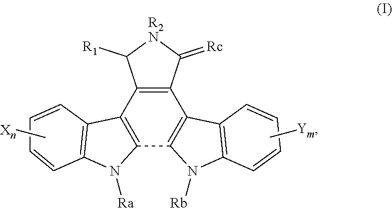

wherein R₁ is H, OH, optionally substituted alkoxy, optionally substituted acyloxy, optionally substituted amino, optionally substituted alkylamino, optionally substituted amido, halogen, optionally substituted C₁₋₆ alkyl, optionally substituted C₂₋₆ alkenyl, optionally substituted C₂₋₆ alkynyl, optionally substituted acyl, optionally substituted alkoxycarbonyl, oxo, thiocarbonyl, optionally substituted carboxy, or ureido;
R₂ is H, optionally substituted C₁₋₆ alkyl, optionally substituted C₂₋₆ alkenyl, optionally substituted C₂₋₆ alkynyl, or optionally substituted acyl;
Rₐ and R_{b} are each, independently, H, optionally substituted C₁₋₆ alkyl, optionally substituted C₂₋₆ alkenyl, or optionally substituted C₂₋₆ alkynyl, optionally substituted and optionally fused aryl, optionally substituted and optionally fused heteroaryl, optionally substituted and optionally fused cycloalkyl, or optionally substituted and optionally fused heterocycloalkyl, or Rₐ and R_{b}, together with the atoms to which they are bound, are joined to form an optionally substituted and optionally fused heterocycloalkyl ring;
R_{c} is O, NR_{d}, or S;
R_{d} is H, optionally substituted C₁₋₆ alkyl, optionally substituted C₂₋₆ alkenyl, or optionally substituted C₂₋₆ alkynyl;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
--- represents a bond that is optionally present;
n is an integer from 0-4; and
m is an integer from 0-4;
or a salt thereof.

### Interfering RNA

Exemplary PKC modulating agents that may be used in conjunction with the compositions and methods of the disclosure include interfering RNA molecules, such as short interfering RNA (siRNA), short hairpin RNA (shRNA), and/or micro RNA (miRNA), that diminish PKC gene expression. Methods for producing interfering RNA molecules are known in the art and are described in detail, for example, in WO 2004/044136 and U.S. Pat. No. 9,150,605, the disclosures of each of which are incorporated herein by reference in their entirety.

### Transduction Using an HDAC Inhibitor

A variety of agents can be used to inhibit histone deacetylases in order to increase the expression of a nucleic acid cassette during viral transduction. Without wishing to be bound by theory, reduced nucleic acid cassette expression from viral vectors may be caused by epigenetic silencing of vector genomes carried out by histone deacetylates. Hydroxamic acids represent a particularly robust class of HDAC inhibitors that inhibit these enzymes by virtue of hydroxamate functionality that binds cationic zinc within the active sites of these enzymes. Exemplary inhibitors include trichostatin A, as well as Vorinostat (N-hydroxy-N′-phenyl-octanediamide, described in Marks et al., Nature Biotechnology 25, 84 to 90 (2007); Stenger, Community Oncology 4, 384-386 (2007), the disclosures of which are incorporated by reference herein). Other HDAC inhibitors include Panobinostat, described in Drugs of the Future 32(4): 315-322 (2007), the disclosure of which is incorporated herein by reference.

Additional examples of hydroxamic acid inhibitors of histone deacetylases include the compounds shown below, described in Bertrand, European Journal of Medicinal Chemistry 45:2095-2116 (2010), the disclosure of which is incorporated herein by reference.

Other HDAC inhibitors that do not contain a hydroxamate substituent have also been developed, including Valproic acid (Gottlicher, et al., EMBO J. 20(24): 6969-6978 (2001) and Mocetinostat (N-(2-Aminophenyl)-4-[[(4-pyridin-3-ylpyrimidin-2-yl)amino]methyl]benzamide, described in Balasubramanian et al., Cancer Letters 280: 211-221 (2009)), the disclosure of each of which is incorporated herein by reference. Other small molecule inhibitors that exploit chemical functionality distinct from a hydroxamate include those described in Bertrand, European Journal of Medicinal Chemistry 45:2095-2116 (2010), the disclosure of which is incorporated herein by reference.

Additional examples of chemical modulators of histone acetylation useful with the compositions and methods of the invention include modulators of HDAC1, HDAC2, HDAC3, HDAC4, HDAC5, HDAC6, HDAC7, HDAC8, HDAC9, HDAC10, Sirt1, Sirt2, and/or HAT, such as butyrylhydroxamic acid, M344, LAQ824 (Dacinostat), AR-42, Belinostat (PXD101), CUDC-101, Scriptaid, Sodium Phenylbutyrate, Tasquinimod, Quisinostat (JNJ-26481585), Pracinostat (SB939), CUDC-907, Entinostat (MS-275), Mocetinostat (MGCD0103), Tubastatin A HCl, PCI-34051, Droxinostat, PCI-24781 (Abexinostat), RGFP966, Rocilinostat (ACY-1215), C1994 (Tacedinaline), Tubacin, RG2833 (RGFP109), Resminostat, Tubastatin A, BRD73954, BG45, 4SC-202, CAY10603, LMK-235, Nexturastat A, TMP269, HPOB, Cambinol, and Anacardic Acid.

In some particular embodiments, the HDAC inhibitor is Scriptaid.

### Transduction Using a Cyclosporine

In some embodiments, therapeutic cells of the disclosure are produced by transducing the cells in the presence of a cyclosporine, such as cyclosporine A (CsA) or cyclosporine H (CsH).

In some embodiments, the concentration of the cyclosporine, when contacted with the cell, is from about 1 μM to about 10 μM (e.g., about 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.1 μM, 3.2 μM, 3.3 μM, 3.4 μM, 3.5 μM, 3.6 μM, 3.7 μM, 3.8 μM, 3.9 μM, 4 μM, 4.1 μM, 4.2 μM, 4.3 μM, 4.4 μM, 4.5 μM, 4.6 μM, 4.7 μM, 4.8 μM, 4.9 μM, 5 μM, 5.1 μM, 5.2 μM, 5.3 μM, 5.4 μM, 5.5 μM, 5.6 μM, 5.7 μM, 5.8 μM, 5.9 μM, 6 μM, 6.1 μM, 6.2 μM, 6.3 μM, 6.4 μM, 6.5 μM, 6.6 μM, 6.7 μM, 6.8 μM, 6.9 μM, 7 μM, 7.1 μM, 7.2 μM, 7.3 μM, 7.4 μM, 7.5 μM, 7.6 μM, 7.7 μM, 7.8 μM, 7.9 μM, 8 μM, 8.1 μM, 8.2 μM, 8.3 μM, 8.4 μM, 8.5 μM, 8.6 μM, 8.7 μM, 8.8 μM, 8.9 μM, 9 μM, 9.1 μM, 9.2 μM, 9.3 μM, 9.4 μM, 9.5 μM, 9.6 μM, 9.7 μM, 9.8 μM, 9.9 μM, or 10 μM).

### Transduction Using an Activator of Prostaglandin E Receptor Signaling

In some embodiments, therapeutic cells of the disclosure are produced by transducing the cells in the presence of an activator of prostaglandin E receptor signaling.

In some embodiments, the activator of prostaglandin E receptor signaling is a small molecule, such as a compound described in WO 2007/112084 or WO 2010/108028, the disclosures of each of which are incorporated herein by reference as they pertain to prostaglandin E receptor signaling activators.

In some embodiments, the activator of prostaglandin E receptor signaling is a small molecule, such as a small organic molecule, a prostaglandin, a Wnt pathway agonist, a cAMP/PI3K/AKT pathway agonist, a Ca²⁺ second messenger pathway agonist, a nitric oxide (NO)/angiotensin signaling agonist, or another compound known to stimulate the prostaglandin signaling pathway, such as a compound selected from Mebeverine, Flurandrenolide, Atenolol, Pindolol, Gaboxadol, Kynurenic Acid, Hydralazine, Thiabendazole, Bicuclline, Vesamicol, Peruvoside, Imipramine, Chlorpropamide, 1,5-Pentamethylenetetrazole, 4-Aminopyridine, Diazoxide, Benfotiamine, 12-Methoxydodecenoic acid, N-Formyl-Met-Leu-Phe, Gallamine, IAA 94, Chlorotrianisene, and or a derivative of any of these compounds.

In some embodiments, the activator of prostaglandin E receptor signaling is a naturally-occurring or synthetic chemical molecule or polypeptide that binds to and/or interacts with a prostaglandin E receptor, typically to activate or increase one or more of the downstream signaling pathways associated with a prostaglandin E receptor.

In some embodiments, the activator of prostaglandin E receptor signaling is selected from the group consisting of prostaglandin (PG) A2 (PGA2), PGB2, PGD2, PGE1 (Alprostadil), PGE2, PGF2, PGI2 (Epoprostenol), PGH2, PGJ2, and derivatives and analogs thereof.

In some embodiments, the activator of prostaglandin E receptor signaling is PGE2 or dmPGE2.

In some embodiments, the activator of prostaglandin E receptor signaling is 15d-PGJ2, delta12-PGJ2, 2-hydroxyheptadecatrienoic acid (HHT), Thromboxane (TXA2 and TXB2), PGI2 analogs, e.g., Iloprost and Treprostinil, PGF2 analogs, e.g., Travoprost, Carboprost tromethamine, Tafluprost, Latanoprost, Bimatoprost, Unoprostone isopropyl, Cloprostenol, Oestrophan, and Superphan, PGE1 analogs, e.g., 11-deoxy PGE1, Misoprostol, and Butaprost, and Corey alcohol-A ([3aa,4a,5,6aa]-(−)-[Hexahydro-4-(hydroxymetyl)-2-oxo-2H-cyclopenta/b/furan-5-yl][1,1′-biphenyl]-4-carboxylate), Corey alcohol-B (2H-Cyclopenta[b]furan-2-on,5-(benzoyloxy)hexahydro-4-(hydroxymethyl)[3aR-(3aa,4a,5,6 aa)]), and Corey diol ((3aR,4S,5R,6aS)-hexahydro-5-hydroxy-4-(hydroxymethyl)-2H-cyclopenta[b]furan-2-one).

In some embodiments, the activator of prostaglandin E receptor signaling is a prostaglandin E receptor ligand, such as prostaglandin E2 (PGE2), or an analogs or derivative thereof. Prostaglandins refer generally to hormone-like molecules that are derived from fatty acids containing 20 carbon atoms, including a 5-carbon ring, as described herein and known in the art. Illustrative examples of PGE2 “analogs” or “derivatives” include, but are not limited to, 16,16-dimethyl PGE2, 16-16 dimethyl PGE2 p-(p-acetamidobenzamido) phenyl ester, I I-deoxy-16,16-dimethyl PGE2, 9-deoxy-9-methylene-16, 16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15 (R)-15-methyl PGE2, 8-iso-15-keto PGE2, 8-iso PGE2 isopropyl ester, 20-hydroxy PGE2, nocloprost, sulprostone, butaprost, 15-keto PGE2, and 19 (R) hydroxy PGE2.

In some embodiments, the activator of prostaglandin E receptor signaling is a prostaglandin analog or derivative having a similar structure to PGE2 that is substituted with halogen at the 9-position (see, e.g., WO 2001/12596, herein incorporated by reference in its entirety), as well as 2-decarboxy-2-phosphinico prostaglandin derivatives, such as those described in US 2006/0247214, herein incorporated by reference in its entirety).

In some embodiments, the activator of prostaglandin E receptor signaling is a non-PGE2-based ligand. In some embodiments, the activator of prostaglandin E receptor signaling is CAY10399, ONO_8815Ly, ONO-AE1-259, or CP-533,536. Additional examples of non-PGE2-based EP2 agonists include the carbazoles and fluorenes disclosed in WO 2007/071456, herein incorporated by reference for its disclosure of such agents. Illustrative examples of non-PGE2-based EP₃ agonist include, but are not limited to, AE5-599, MB28767, GR 63799X, ONO-NT012, and ONO-AE-248. Illustrative examples of non-PGE2-based EP4 agonist include, but are not limited to, ONO-4819, APS-999 Na, AH23848, and ONO-AE 1-329. Additional examples of non-PGE2-based EP4 agonists can be found in WO 2000/038663; U.S. Pat. Nos. 6,747,037; and 6,610,719, each of which are incorporated by reference for their disclosure of such agonists In some embodiments, the activator of prostaglandin E receptor signaling is a Wnt agonist.

Illustrative examples of Wnt agonists include, but are not limited to, Wnt polypeptides and glycogen synthase kinase 3 (GSK3) inhibitors. Illustrative examples of Wnt polypeptides suitable for use as compounds that stimulate the prostaglandin EP receptor signaling pathway include, but are not limited to, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or biologically active fragments thereof. GSK3 inhibitors suitable for use as agents that stimulate the prostaglandin EP receptor signaling pathway bind to and decrease the activity of GSK3a, or GSK3. Illustrative examples of GSK3 inhibitors include, but are not limited to, BIO (6-bromoindirubin-3′-oxime), LiCl, Li₂CO₃, or other GSK-3 inhibitors, as exemplified in U.S. Pat. Nos. 6,057,117 and 6,608,063, as well as US 2004/0092535 and US 2004/0209878, and ATP-competitive, selective GSK-3 inhibitors CHIR-911 and CHIR-837 (also referred to as CT-99021/CHIR-99021 and CT-98023/CHIR-98023, respectively) (Chiron Corporation (Emeryville, CA)). The structure of CHIR-99021 is

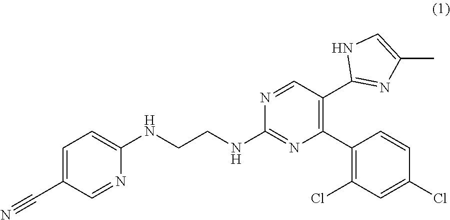 [SMILES: Cc1c[nH]c(-c2cnc(NCCNc3ccc(C#N)cn3)nc2-c2ccc(Cl)cc2Cl)n1]

or a salt thereof.
   The structure of CHIR-98023 is

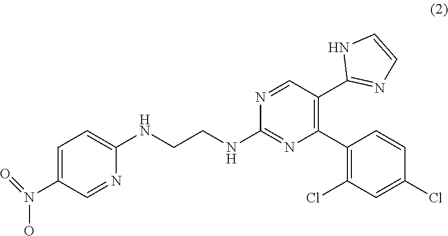

or a salt thereof.

In some embodiments, method further includes contacting the cell with a GSK3 inhibitor.

In some embodiments, the GSK3 inhibitor is CHIR-99021 or CHIR-98023.

In some embodiments, the GSK3 inhibitor is Li₂CO₃.

In some embodiments, the activator of prostaglandin E receptor signaling is an agent that increases signaling through the cAMP/P13K/AKT second messenger pathway, such as an agent selected from the group consisting of dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, pituitary adenylate cyclase activating polypeptide (PACAP), and vasoactive intestinal polypeptide (VIP), and derivatives of these agents.

In some embodiments, the activator of prostaglandin E receptor signaling is an agent that increases signaling through the Ca²⁺ second messenger pathway, such as an agent selected from the group consisting of Bapta-AM, Fendiline, Nicardipine, and derivatives of these agents.

In some embodiments, the activator of prostaglandin E receptor signaling is an agent that increases signaling through the NO/Angiotensin signaling, such as an agent selected from the group consisting of L-Arg, Sodium Nitroprusside, Sodium Vanadate, Bradykinin, and derivatives thereof.

### Transduction Using a Polycationic Polymer

In some embodiments, therapeutic cells of the disclosure are produced by transducing the cells in the presence of a polycationic polymer. In some embodiments, the polycationic polymer is polybrene, protamine sulfate, polyethylenimine, or a polyethylene glycol/poly-L-lysine block copolymer.

In some embodiments, the polycationic polymer is protamine sulfate.

In some embodiments, the cell is further contacted with an expansion agent during the transduction procedure. The cell may be, for example, a hematopoietic stem cell and the expansion agent may be a hematopoietic stem cell expansion agent, such as a hematopoietic stem cell expansion agent known in the art or described herein.

### Additional Transduction Enhancers

In some embodiments of the methods described herein, during the transduction procedure, the cell is further contacted with an agent that inhibits mTOR signaling. The agent that inhibits mTOR signaling may be, for example, rapamycin, among other suppressors of mTOR signaling.

Additional transduction enhancers that may be used in conjunction with the compositions and methods of the disclosure include, for example, tacrolimus and vectorfusin.

### Spinoculation

In some embodiments of the disclosure, a cell targeted for transduction may be spun e.g., by centrifugation, while being cultured with a viral vector (e.g., in combination with one or more additional agents described herein). This “spinoculation” process may occur with a centripetal force of, e.g., from about 200×g to about 2,000×g. The centripetal force may be, e.g., from about 300×g to about 1,200×g (e.g., about 300×g, 400×g, 500×g, 600×g, 700×g, 800×g, 900×g, 1,000×g, 1,100×g, or 1,200×g, or more). In some embodiments, the cell is spun for from about 10 minutes to about 3 hours (e.g., about 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or more). In some embodiments, the cell is spun at room temperature, such as at a temperature of about 25° C.

Exemplary transduction protocols involving a spinoculation step are described, e.g., in Millington et al., PLoS One 4:e6461 (2009); Guo et al., Journal of Virology 85:9824-9833 (2011); O'Doherty et al., Journal of Virology 74:10074-10080 (2000); and Federico et al., Lentiviral Vectors and Exosomes as Gene and Protein Delivery Tools, Methods in Molecular Biology 1448, Chapter 4 (2016), the disclosures of each of which are incorporated herein by reference.

### Viral Vectors for Expression

Viral genomes provide a rich source of vectors that can be used for the efficient delivery of exogenous genes into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the nucleic acid cassette. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the nucleic acid cassette of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3-splice site (SA), elongation factor (EF) 1-alpha promoter and 3-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of nucleic acid cassette expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Methods of Ex Vivo Transfection

One platform that can be used to achieve therapeutically effective intracellular concentrations of one or more proteins described herein in mammalian cells is via the stable expression of genes encoding these agents (e.g., by integration into the nuclear or mitochondrial genome of a mammalian cell). These genes are polynucleotides that encode the primary amino acid sequence of the corresponding protein. In order to introduce such exogenous genes into a mammalian cell, these genes can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

Genes encoding therapeutic proteins of the disclosure can also be introduced into mammalian cells by targeting a vector containing a gene encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such, a construct can be produced using methods well known to those of skill in the field.

Recognition and binding of the polynucleotide encoding one or more therapeutic proteins of the disclosure by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol., 3:73, online publication, the disclosure of which is incorporated herein by reference.

Once a polynucleotide encoding one or more therapeutic proteins has been incorporated into the nuclear DNA of a mammalian cell, transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode one or more therapeutic proteins and additionally include a mammalian enhancer sequence. Many enhancer sequences are now known from mammalian genes, and examples are enhancers from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). Further examples of enhancers for use in the compositions and methods described herein include CNS1, CNS2, CNS3, and CNS0 enhancers, as described in Lee et al., Exp. Mol. Med. 50(3):e456 (2018); Kawakami et al., Immunity. 54(5):947-961 (2021); Kim et al., J. Exp. Med. 204(7):1543-51 (2007); Zheng et al., Nature. 463(7282):808-12 (2010); Tone et al., Nat. Immunol. 9(2):194-202 (2008); and Dikiy et al., Immunity. 54(5):931-946 (2021).

### Cells for Expression and Delivery

Cells that may be used in conjunction with the compositions and methods described herein include cells that are capable of undergoing further differentiation. For example, one type of cell that can be used in conjunction with the compositions and methods described herein is a pluripotent cell, which possesses the ability to develop into more than one differentiated cell type. An example of a pluripotent cell includes a pluripotent hematopoietic cell, which has the ability to develop into more than one differentiated cell type of the hematopoietic lineage. Examples of pluripotent hematopoietic cells that may be used in conjunction with the compositions and methods described herein include HSCs, HPCs, ESCs, iPSCs, lymphoid progenitor cells, and CD34+ cells.

Cells that may be used in conjunction with the compositions and methods described herein include hematopoietic stem cells and hematopoietic progenitor cells. Hematopoietic stem cells (HSCs) are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs and other pluripotent progenitors can be obtained from blood products. A blood product is a product obtained from the body or an organ of the body containing cells of hematopoietic origin. Such sources include unfractionated bone marrow, umbilical cord, placenta, peripheral blood, or mobilized peripheral blood. All of the aforementioned crude or unfractionated blood products can be enriched for cells having HSC or lymphoid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, NY). Lymphoid progenitor cells can also be isolated based on the markers they express. Unfractionated blood products can be obtained directly from a donor or retrieved from cryopreservative storage. HSCs and lymphoid progenitor cells can also be obtained from by differentiation of ES cells, PS cells or other reprogrammed mature cell types.

Cells that may be used in conjunction with the compositions and methods described herein include allogeneic cells and autologous cells. When allogeneic cells are used, the cells may optionally be HLA-matched to the subject receiving a cell treatment.

Cells that may be used in conjunction with the compositions and methods described herein include CD34+/CD90+ cells and CD34+/CD164+ cells. These cells may contain a higher percentage of HSCs. These cells are described in Radtke et al. Sci. Transl. Med. 9: 1-10, 2017, and Pellin et al. Nat. Comm. 1-: 2395, 2019, the disclosures of each of which are hereby incorporated by reference in their entirety.

The cells described herein and above may be genetically modified so as to express the autoantigen-binding protein (e.g., single-chain protein (e.g., chimeric antigen receptor or single-chain antibody fragment) or multi-chain protein (e.g., a full-length antibody, a dual-variable immunoglobulin domain, a diabody, a triabody, an antibody-like protein scaffold, a Fab fragment, or a F(ab′)₂ molecule)) described herein using, for example, a variety of methodologies as described herein. Once the cells have been adapted to express physiological or suitable levels of the autoantigen-binding protein, these cells have therapeutic utility, and are referred to herein as “therapeutic cells of the disclosure.”

### Gene Editing Techniques

In addition to the above, a variety of tools have been developed that can be used for the incorporation of a gene of interest into a cell, such as a pluripotent cell (e.g., a pluripotent hematopoietic cell). One such method that can be used for incorporating polynucleotides encoding target genes into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another useful tool for the integration of target genes into the genome of a cell (e.g., a pluripotent hematopoietic cell) is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and a CRISPR-associated protein (Cas; e.g., Cas9 or Cas12a). This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas nuclease to this site. In this manner, highly site-specific Cas-mediated DNA cleavage can be engendered in a foreign polynucleotide because the interaction that brings Cas within close proximity of the target DNA molecule is governed by RNA: DNA hybridization. As a result, one can design a CRISPR/Cas system to cleave any target DNA molecule of interest. This technique has been exploited in order to edit eukaryotic genomes (Hwang et al. Nature Biotechnology 31:227 (2013), the disclosure of which is incorporated herein by reference) and can be used as an efficient means of site-specifically editing pluripotent stem cell genomes in order to cleave DNA prior to the incorporation of a gene encoding a target gene. The use of CRISPR/Cas to modulate gene expression has been described in, e.g., WO 2017/182881 and U.S. Pat. No. 8,697,359, the disclosures of each of which are incorporated herein by reference.

Alternative methods for site-specifically cleaving genomic DNA prior to the incorporation of a gene of interest in a pluripotent hematopoietic cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (2010); and in Joung et al. Nature Reviews Molecular Cell Biology 14:49 (2013), the disclosures of each of which are incorporated herein by reference.

Additional genome editing techniques that can be used to incorporate polynucleotides encoding target genes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

Agents that Promote Pluripotent Cell Mobilization

In some embodiments of the disclosure, prior to isolation of a pluripotent cell (e.g., a pluripotent hematopoietic cell) from the subject being treated for an autoimmune disease (e.g., in the case of an autologous cell population) or from a donor (e.g., in the case of an allogeneic cell population), the subject or donor is administered one or more mobilization agents that stimulate the migration of pluripotent hematopoietic cells (e.g., CD34+ HSCs and HPCs) from a stem cell niche, such as the bone marrow, to peripheral circulation. Exemplary cell mobilization agents that may be used in conjunction with the compositions and methods of the disclosure are described herein and known in the art. For example, the mobilization agent may be a C-X-C motif chemokine receptor (CXCR) 2 (CXCR2) agonist. The CXCR2 agonist may be Gro-beta, or a truncated variant thereof. Gro-beta and variants thereof are described, for example, in U.S. Pat. Nos. 6,080,398; 6,447,766; and 6,399,053, the disclosures of each of which are incorporated herein by reference in their entirety. Additionally or alternatively, the mobilization agent may include a CXCR4 antagonist, such as plerixafor or a variant thereof. Plerixafor and structurally similar compounds are described, for example, in U.S. Pat. Nos. 6,987,102; 7,935,692; and 7,897,590, the disclosures of each of which are incorporated herein by reference. Additionally or alternatively, the mobilization agent may include granulocyte colony-stimulating factor (G-CSF). The use of G-CSF as an agent to induce mobilization of pluripotent hematopoietic cells (e.g., CD34+ HSCs and/or HPCs) from a stem cell niche to peripheral circulation is described, for example, in US 2010/0178271, the disclosure of which is incorporated herein by reference in its entirety.

Agents that Enhance Cellular Engraftment

In some embodiments, prior to administration of the population of cells (e.g., CD34+ cells), as described herein, to the patient, the patient may be administered an agent that ablates an endogenous population of CD34+ cells, allowing the administered CD34+ cells to engraft in the patient. Examples of conditioning agents include myeloablative conditioning agents, which deplete a wide variety of hematopoietic cells in a patient. For instance, that patient may be pre-treated with an alkylating agent, such as a nitrogen mustard (e.g., bendamustine, chlorambucil, cyclophosphamide, ifosfamide, mechlorethamine, or melphalan), a nitrosourea (e.g., carmustine, lomustine, or streptozocin), an alkyl sulfonate (e.g., busulfan), a triazine (e.g., dacarbazine or temozolomide), or an ethylenimine (e.g., altretamine or thiotepa). In some embodiments, the patient is administered a conditioning agent that selectively ablates a specific population of endogenous cells, such as a population of endogenous CD34+ HSCs or HPCs.

In some embodiments, the conditioning agent includes an antibody or antigen-biding fragment thereof. The antibody or antigen-binding fragment thereof may bind to CD117, HLA-DR, CD34, CD90, CD45, or CD133 (e.g., CD117). The antibody or antigen-binding fragment thereof may be conjugated to a cytotoxin.

In some embodiments, the patient is pre-treated with an activator of prostaglandin E receptor signaling in order to help facilitate the engraftment of administered cells. The prostaglandin E receptor signaling activator may be, for example, selected from the group consisting of prostaglandin (PG) A2 (PGA2), PGB2, PGD2, PGE1 (Alprostadil), PGE2, PGF2, PGI2 (Epoprostenol), PGH2, PGJ2, and derivatives and analogs thereof.

In some embodiments, the activator of prostaglandin E receptor signaling used to help facilitate engraftment of a cell is PGE2 or dmPG2.

In some embodiments, the activator of prostaglandin E receptor signaling used to help facilitate engraftment of a cell is 15d-PGJ2, delta12-PGJ2, 2-hydroxyheptadecatrienoic acid (HHT), Thromboxane (TXA2 and TXB2), PGI2 analogs, e.g., Iloprost and Treprostinil, PGF2 analogs, e.g., Travoprost, Carboprost tromethamine, Tafluprost, Latanoprost, Bimatoprost, Unoprostone isopropyl, Cloprostenol, Oestrophan, and Superphan, PGE1 analogs, e.g., 11-deoxy PGE1, Misoprostol, and Butaprost, and Corey alcohol-A ([3aa,4a,5,6aa]-(−)-[Hexahydro-4-(hydroxymetyl)-2-oxo-2H-cyclopenta/b/furan-5-yl][1,1′-biphenyl]-4-carboxylate), Corey alcohol-B (2H-Cyclopenta[b]furan-2-on,5-(benzoyloxy)hexahydro-4-(hydroxymethyl)[3aR-(3aa,4a,5,6aa)]), and Corey diol ((3aR,4S,5R,6aS)-hexahydro-5-hydroxy-4-(hydroxymethyl)-2H-cyclopenta[b]furan-2-one).

In some embodiments, the activator of prostaglandin E receptor signaling used to help facilitate engraftment of a cell is a prostaglandin E receptor ligand, such as prostaglandin E2 (PGE2), or an analogs or derivative thereof. Prostaglandins refer generally to hormone-like molecules that are derived from fatty acids containing 20 carbon atoms, including a 5-carbon ring, as described herein and known in the art. Illustrative examples of PGE2 “analogs” or “derivatives” include, but are not limited to, 16,16-dimethyl PGE2, 16-16 dimethyl PGE2 p-(p-acetamidobenzamido) phenyl ester, I I-deoxy-16,16-dimethyl PGE2, 9-deoxy-9-methylene-16, 16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15 (R)-15-methyl PGE2, 8-iso-15-keto PGE2, 8-iso PGE2 isopropyl ester, 20-hydroxy PGE2, nocloprost, sulprostone, butaprost, 15-keto PGE2, and 19 (R) hydroxy PGE2.

In some embodiments, the activator of prostaglandin E receptor signaling used to help facilitate engraftment of a cell is a prostaglandin analog or derivative having a similar structure to PGE2 that is substituted with halogen at the 9-position (see, e.g., WO 2001/12596, herein incorporated by reference in its entirety), as well as 2-decarboxy-2-phosphinico prostaglandin derivatives, such as those described in US 2006/0247214, herein incorporated by reference in its entirety).

In some embodiments, the activator of prostaglandin E receptor signaling used to help facilitate engraftment of a cell is a non-PGE2-based ligand. In some embodiments, the activator of prostaglandin E receptor signaling used to help facilitate engraftment of a cell is CAY10399, ONO_8815Ly, ONO-AE1-259, or CP-533,536. Additional examples of non-PGE2-based EP2 agonists include the carbazoles and fluorenes disclosed in WO 2007/071456, herein incorporated by reference for its disclosure of such agents. Illustrative examples of non-PGE2-based EP₃ agonist include, but are not limited to, AE5-599, MB28767, GR 63799X, ONO-NT012, and ONO-AE-248. Illustrative examples of non-PGE2-based EP4 agonist include, but are not limited to, ONO-4819, APS-999 Na, AH23848, and ONO-AE 1-329. Additional examples of non-PGE2-based EP4 agonists can be found in WO 2000/038663; U.S. Pat. Nos. 6,747,037; and 6,610,719, each of which are incorporated by reference for their disclosure of such agonists.

In some embodiments, the activator of prostaglandin E receptor signaling used to help facilitate engraftment of a cell is a Wnt agonist. Illustrative examples of Wnt agonists include, but are not limited to, Wnt polypeptides and glycogen synthase kinase 3 (GSK3) inhibitors. Illustrative examples of Wnt polypeptides suitable for use as compounds that stimulate the prostaglandin EP receptor signaling pathway include, but are not limited to, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or biologically active fragments thereof. GSK3 inhibitors suitable for use as agents that stimulate the prostaglandin EP receptor signaling pathway bind to and decrease the activity of GSK3a, or GSK3. Illustrative examples of GSK3 inhibitors include, but are not limited to, BIO (6-bromoindirubin-3′-oxime), LiCl, Li₂CO₃ or other GSK-3 inhibitors, as exemplified in U.S. Pat. Nos. 6,057,117 and 6,608,063, as well as US 2004/0092535 and US 2004/0209878, and ATP-competitive, selective GSK-3 inhibitors CHIR-911 and CHIR-837 (also referred to as CT-99021/CHIR-99021 and CT-98023/CHIR-98023, respectively) (Chiron Corporation (Emeryville, CA)).

In some embodiments, the activator of prostaglandin E receptor signaling used to help facilitate engraftment of a cell is an agent that increases signaling through the cAMP/P13K/AKT second messenger pathway, such as an agent selected from the group consisting of dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, pituitary adenylate cyclase activating polypeptide (PACAP), and vasoactive intestinal polypeptide (VIP), and derivatives of these agents.

In some embodiments, the activator of prostaglandin E receptor signaling used to help facilitate engraftment of a cell is an agent that increases signaling through the Ca²⁺ second messenger pathway, such as an agent selected from the group consisting of Bapta-AM, Fendiline, Nicardipine, and derivatives of these agents.

In some embodiments, the activator of prostaglandin E receptor signaling used to help facilitate engraftment of a cell is an agent that increases signaling through the NO/Angiotensin signaling, such as an agent selected from the group consisting of L-Arg, Sodium Nitroprusside, Sodium Vanadate, Bradykinin, and derivatives thereof.

### Routes of Administration

The compositions described herein may be administered to a patient (e.g., a human patient suffering from an autoimmune disease) by one or more of a variety of routes, such as intravenously or by means of a bone marrow transplant. The most suitable route for administration in any given case may depend on the particular composition administered, the patient, pharmaceutical formulation methods, administration methods (e.g., administration time and administration route), the patient's age, body weight, sex, severity of the diseases being treated, the patient's diet, and the patient's excretion rate. Multiple routes of administration may be used to treat a single patient at one time, or the patient may receive treatment via one route of administration first and receive treatment via another route of administration during a second appointment, e.g., 1 week later, 2 weeks later, 1 month later, 6 months later, or 1 year later. Compositions may be administered to a subject once, or cells may be administered one or more times (e.g., 2-10 times) per week, month, or year.

### Selection of Donor Cells

In some embodiments, the patient undergoing treatment is the donor that provides cells (e.g., pluripotent cells, such as pluripotent hematopoietic cells (e.g., CD34+ hematopoietic stem or progenitor cells)) that are subsequently modified to express one or more therapeutic proteins of the disclosure before being re-administered to the patient. In such cases, withdrawn cells (e.g., hematopoietic stem or progenitor cells) may be re-infused into the subject following, for example, incorporation of a nucleic acid cassette encoding an autoantigen-binding protein, such that the cells may subsequently home to hematopoietic tissue and establish productive hematopoiesis, thereby populating or repopulating a line of cells that is defective or deficient in the patient. In cases in which the patient undergoing treatment also serves as the cell donor, the transplanted cells (e.g., hematopoietic stem or progenitor cells) are less likely to undergo graft rejection. This stems from the fact that the infused cells are derived from the patient and express the same HLA class I and class II antigens as expressed by the patient. Alternatively, the patient and the donor may be distinct. In some embodiments, the patient and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign, and are thus less likely to mount an immune response against the transplant. Exemplary HLA-matched donor-recipient pairs are donors and recipients that are genetically related, such as familial donor-recipient pairs (e.g., sibling donor-recipient pairs). In some embodiments, the patient and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Pharmaceutical Compositions and Dosing

In cases in which a patient is administered a population of cells that together express one or more therapeutic proteins of the disclosure, the number of cells administered may depend, for example, on the expression level of the desired protein(s), the patient, pharmaceutical formulation methods, administration methods (e.g., administration time and administration route), the patient's age, body weight, sex, severity of the disease being treated, and whether or not the patient has been treated with agents to ablate endogenous pluripotent cells (e.g., pluripotent hematopoietic cells, such as endogenous CD34+ cells, hematopoietic stem or progenitor cells, among others). The number of cells administered may be, for example, from 1×10⁶ cells/kg to 1×10¹² cells/kg, or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of pluripotent cells may be administered in any suitable dosage form.

Cells may be admixed with one or more pharmaceutically acceptable carriers, diluents, and/or excipients. Exemplary carriers, diluents, and excipients that may be used in conjunction with the compositions and methods of the disclosure are described, e.g., in Remington: The Science and Practice of Pharmacy (2012, 22nd ed.) and in The United States Pharmacopeia: The National Formulary (2015, USP 38 NF 33).

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Designing a Lentiviral Vector Construct to Allow Expression of Chimeric Antigen Receptors (CAR) Under the Control of a Constitutive Promoter for Proof of Concept (PoC) Studies

### Objective

The objective of this study was to design a lentiviral vector construct to allow expression of a chimeric antigen receptor (CAR) under the control of a constitutive promoter for PoC studies. The lentiviral vector construct was designed to express a CAR that specifically binds a desired antigen, such that it may be used to impart antigen-binding capacity to a cell (e.g., a hematopoietic stem cell-derived regulatory T cell, as described herein).

### Materials and Methods

A lentiviral vector construct was designed by incorporating the following elements: a Rev response element (RRE); central polypurine tract (cPPT); elongation factor 1a short binding sequence (EFS) promoter; a Kozak consensus sequence; a coding region encoding single chain variable fragments (including a variable light chain (VL), linker, variable heavy chain (VH), and second generation CAR signaling domains (CD28 hinge domain, CD28 transmembrane (TM) and signal domains, and CD3(signal domain)); and Woodchuck hepatitis virus post transcriptional regulatory element (WPRE). Single chain variable fragments were generated by linking heavy and light chain sequences from antibodies with known antigen specificity. A His-tag was introduced to facilitate detection of CARs. Second generation CAR signaling domains were chosen for compatibility with regulatory T cell function.

### Results

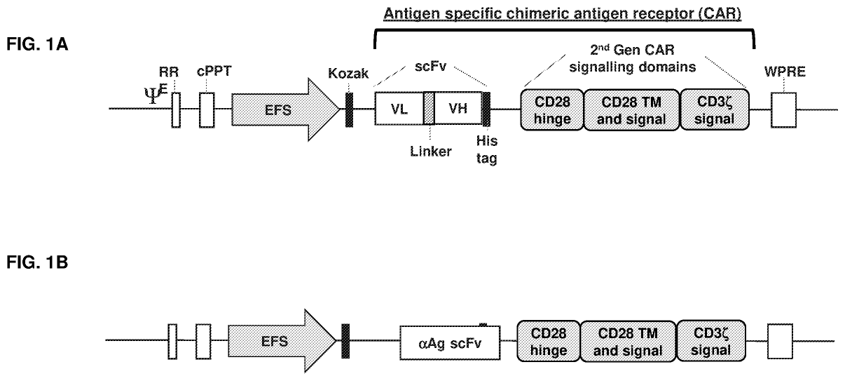

The elements described above are shown in FIG. 1A, which provides an illustration of exemplary components that may be incorporated into a lentiviral vector construct of the disclosure. Shown in FIG. 1B is an exemplary lentiviral vector construct that was produced using the elements discussed above. As FIG. 1B shows, the lentiviral vector construct that was produced included an RRE, cPPT, EFS promoter, Kozak consensus sequence, a coding sequence encoding an scFv having antigen specificity and second-generation CAR signaling domains, as well as a WPRE. The construct produced in FIG. 1B was subsequently used in the PoC studies described in Examples 2-9, below. For the purpose of these Examples, an scFv with specificity for an irrelevant antigen (Ag) was selected to allow optimisation of in vitro assays and to test the safety and function of CAR biology in vivo.

### Example 2. Expressing Antigen-Specific CAR in a Human T Cell Line Followed by Assessment of the Level of Expression Using Flow Cytometry

### Objective

The objective of this study was to express an antigen-specific CAR in a human T cell line and then assess the level of expression using flow cytometry.

### Materials and Methods

Jurkat T cells were either untransduced or transduced with a lentiviral vector (multiplicity of infection, MOI5) to express an antigen-specific CAR (aAg-CAR). After 72 hours, CAR expression was assessed by flow cytometry (FC) by incubating cells with 50,000 μg/ml biotinylated CAR ligand (whole protein) before staining with a streptavidin-PE conjugate. A titration of CAR ligand was then performed to assess receptor expression, quantified as mean fluorescence intensity (MFI).

A library of Jurkat T cells expressing different levels of Ag-specific CAR was generated using MOI titration ranging from 0 to 5. Vector copy number (VCN) was measured by droplet digital PCR (ddPCR) and % CAR⁺ cells as a measure of the MOI used was quantified using FC.

Jurkat T cells were transduced with a lentiviral vector with different MOIs ranging from 0 to 5 to express an antigen-specific CAR (aAg-CAR). After 72 hours, CAR expression as a measure of the MOI used was assessed by FC by incubating cells with either 0 pg/ml or 50,000 μg/ml biotinylated CAR ligand (whole protein) before staining with a streptavidin-PE conjugate.

### Results

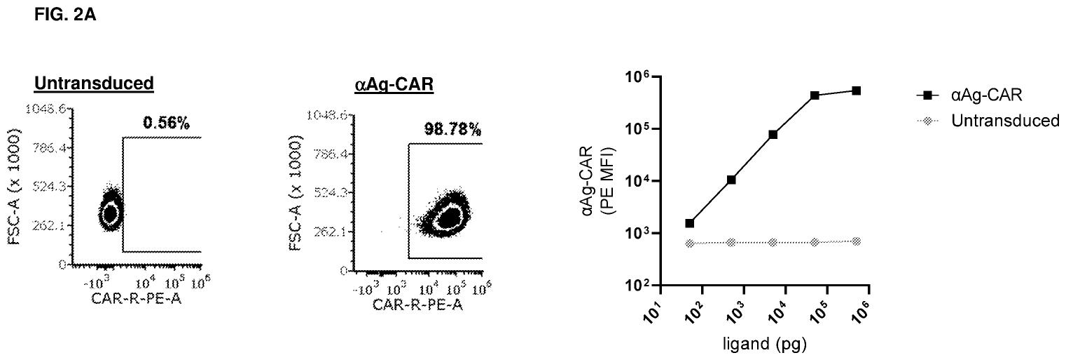

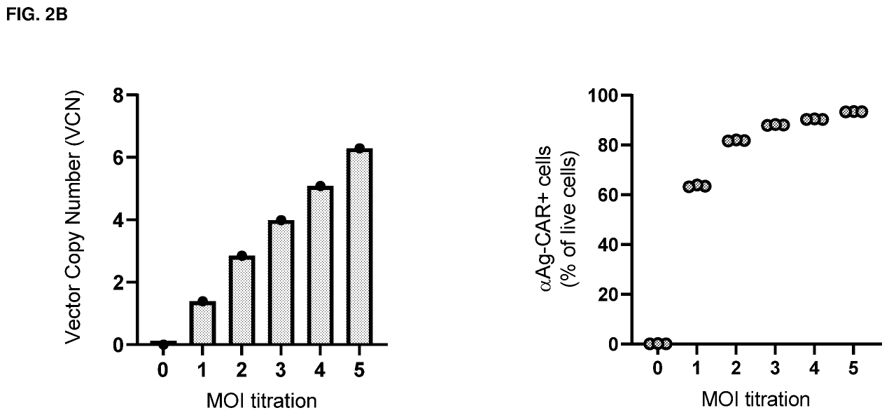

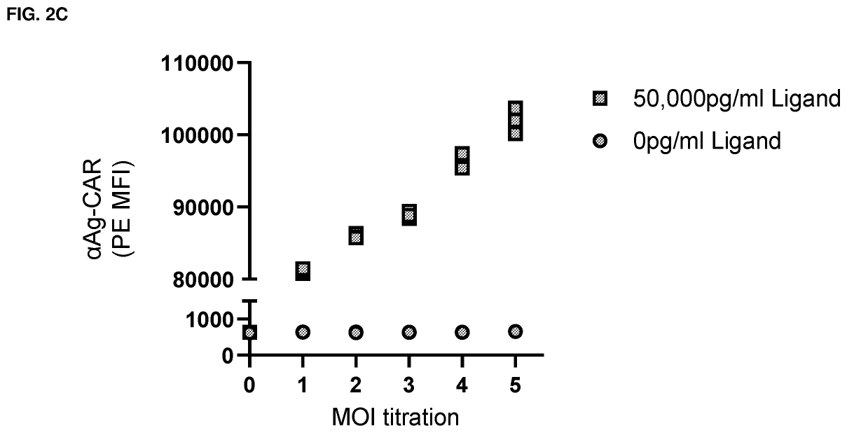

In result, we observed that Jurkat T cells that were transduced with the lentiviral vector (MOI5), expressed the antigen-specific CAR (aAg-CAR) (FIG. 2A). FC plots were gated on live Jurkat T cells, depicting untransduced cells (negative control) and transduced cells, where the proportion of CAR expressing cells was determined by gating on cells with surface-bound CAR ligand (CAR-R-PE-A). FIG. 2A also shows that a titration of CAR ligand in pg was used to assess receptor expression, quantified by way of mean fluorescence intensity (PE MFI). It was observed that transduced cells had a higher PE MFI compared to untransduced cell across most ligand concentrations (ranging from slightly less than 10² to 10⁶). The PE MFI for transduced cells also increased with increasing ligand concentration.

We also observed that transgene vector copy number (VCN) in Jurkat T cells, which was measured by ddPCR (FIG. 2B) increased with increasing MOI titration ranging from 0 to 5. % CAR⁺ cells as a measure of MOI titration was quantified in the library of Jurkat T cells generated using MOI titration, and it was observed that the % of live aAg-CAR+ cells increased with increasing MOI titration. Furthermore, we observed via FC (FIG. 2C) that PE MFI, which is a measure of CAR expression, was higher at 50,000 μg/ml concentration of the ligand compared to 0 pg/ml of the ligand at all MOI values ranging from 1 to 5.

### Example 3. Confirming the Function of Antigen-Specific CAR Function In Vitro in a Human T Cell Line

### Objective

The objective of this study was to confirm the function of antigen-specific CAR function in vitro in a human T cell line after their expression.

### Materials and Methods

Jurkat T cells were transduced with lentiviral vectors to express different levels of aAg-CAR (transduction efficiencies shown in FIG. 2) and were treated with increasing amounts of CAR ligand in vitro for 24 hrs. Following that, FC was performed to assess CAR function. CAR function was measured as the levels of expressed T cell activation markers, CD69 and CD25 at different MOI titrations ranging from 0 to 5. In addition, supernatants from cultured Jurkat T cells were collected and assessed for IL-2 production by enzyme-linked immunosorbent assay (ELISA).

### Results

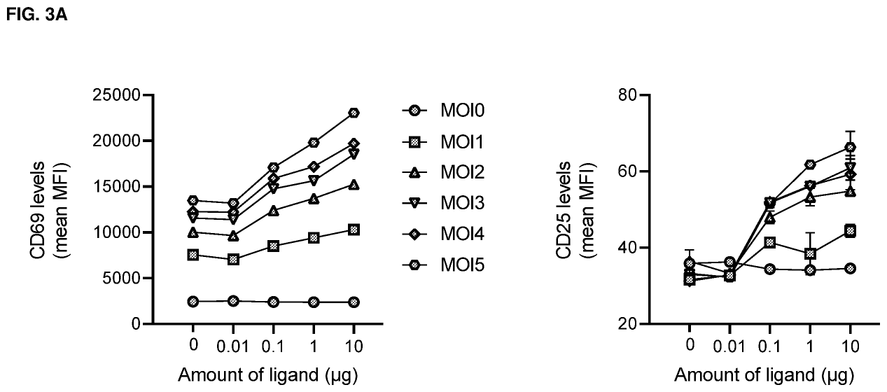

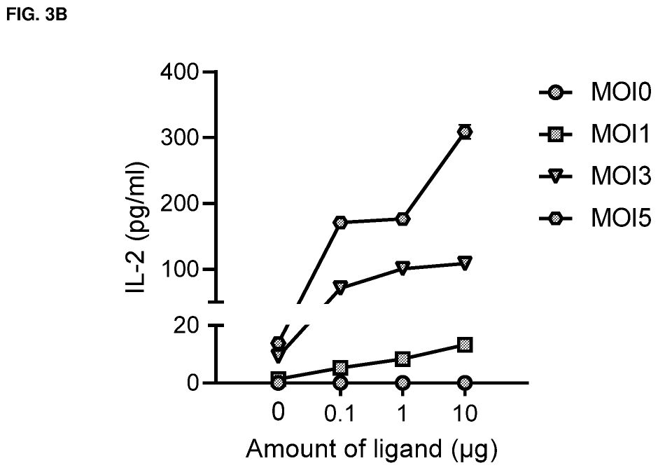

In result, we observed that CD69 expression measured here as mean MFI via FC (FIG. 3A, left graph) increased with increasing MOI values (ranging from 1 to 5) as well as with increasing ligand concentration (0.01 μg to 10 μg). We also observed that CD25 expression increased with increasing MOI values (ranging from 1 to 5) as well as with increasing ligand concentration (0.01 μg to 10 μg). Additionally, as is shown in FIG. 3B, the level of IL-2 produced by cultured Jurkat cells, measured via ELISA, increased with increasing MOI values (1,3,5) as well as with increasing ligand concentration (0 μg to 10 μg). Taken together, these data confirm antigen-specific CAR functionality following transduction in human T cells.

### Example 4. Expressing Antigen-Specific CAR in Primary Murine T Cells Followed by Assessment of the Level of Expression Using Flow Cytometry and the Level of Function Using Flow Cytometry and Enzyme-Linked Immunosorbent Assay

### Objective

The objective of this study was to express antigen-specific CAR in primary murine T cells followed by assessment of the level of expression using flow cytometry and the level of function using flow cytometry and enzyme-linked immunosorbent assay.

### Materials and Methods

Purified, CD4⁺CD25⁻ naïve splenic T cells were activated in vitro using CD3/CD28 microbeads before addition of lentiviral vectors (MOI10) for expression of aAg-CAR. After 72 hrs, expression of aAg-CAR was confirmed by FC analysis. Some cells were untransduced to serve as negative controls.

Transduced CD4⁺CD25⁻ T cells were treated with increasing concentrations of CAR ligand in vitro for 48 hrs. T cell activation was assessed by measuring CD69 and CD25 expression by FC, quantified as MFI. Supernatants from cultured cells were assessed in parallel via ELISA for IL-2 secretion.

### Results

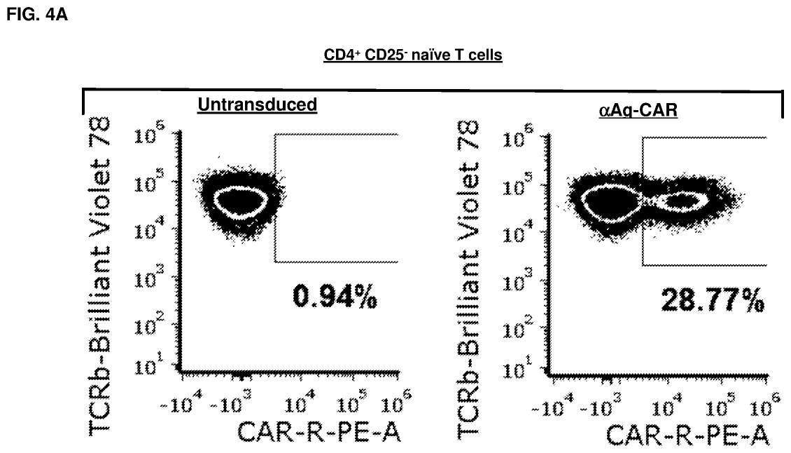

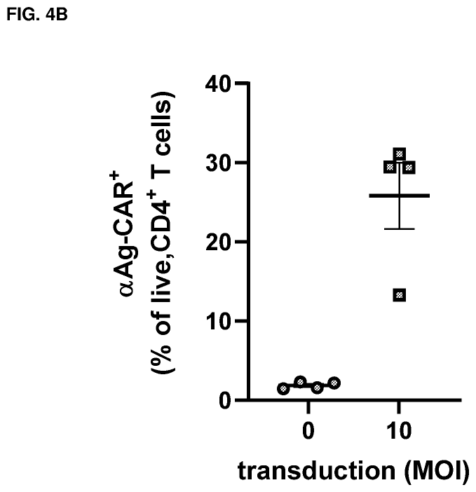

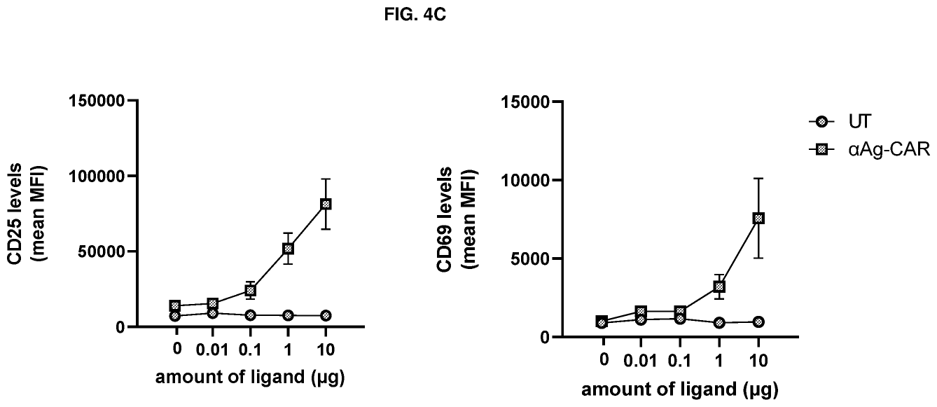

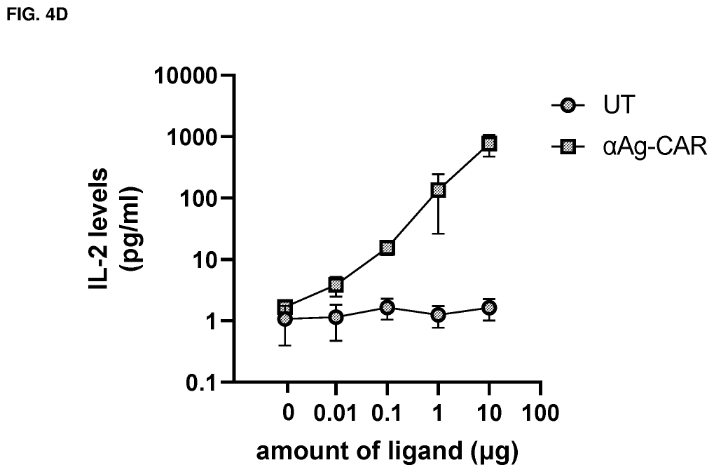

In result, we observed that transduced CD4⁺CD25⁻ naïve splenic T cells showed higher ligand binding compared to untransduced cells as evident from the presence of the FC contour (28.77%) in the top right quadrant of transduced cells (FIG. 4A). Here, TCRb Brilliant Violet 78 concentration is used to identify T cells and the proportion of CAR expressing cells was determined by gating on cells with surface-bound CAR ligand (CAR-R-PE-A). Plots are gated on live, CD4⁺ T cells. Untransduced cells were used as negative controls. The % of αAg-CAR⁺ transduced cells were quantified as % of live, CD4⁺ T cells (n=4) (FIG. 4B) and it was observed that the % of live, αAg-CAR⁺ CD4⁺ T cells was higher at transduction MOI10 and almost negligible at transduction MOI0 (untransduced cells), suggesting that the increase in the % of live, αAg-CAR⁺ CD4⁺ T cells was a direct result of transduction with the lentiviral vector encoding αAg-CAR.

We further observed that CD25 expression, measured here as mean MFI via FC (FIG. 4C, left graph), was higher in transduced cells (square) than in untransduced cells (circle) and it increased with increasing ligand concentration (0.01 μg to 10 μg). We also observed that CD69 expression, measured here as mean MFI via FC (FIG. 4C, right graph), was higher in transduced cells (square) than in untransduced cells (circle) and it increased with increasing ligand concentration (0.1 μg to 10 μg). Moreover, the level of IL-2 produced by cultured cells measured via ELISA (FIG. 4D) increased with transduction as well as with increasing ligand concentration (0 μg to 100 μg). Taken together, these data demonstrate that transduction of primary murine T cells results in the expression of a functional antigen-specific CAR.

### Example 5. Expressing Antigen-Specific CAR in Primary Murine Regulatory T Cells Followed by Assessment of the Level of Expression Using Flow Cytometry and the Level of Function Using Enzyme-Linked Immunosorbent Assay

### Objective

The objective of this study was to express antigen-specific CAR in primary murine regulatory T cells followed by assessment of the level of expression using flow cytometry and the level of function using enzyme-linked immunosorbent assay.

### Materials and Methods

Purified, CD4⁺CD25⁺ regulatory T cells (Tregs), were activated in vitro using CD3/CD28 microbeads before lentiviral transduction (MOI10) for expressing αAg-CAR. After 72 hrs, FC was performed to confirm expression of αAg-CAR.

Transduced CD4⁺CD25+ Tregs were cultured for 48 hrs in media alone or 10 μg CAR ligand. Supernatants were collected and IL-10 secretion quantified was ELISA.

### Results

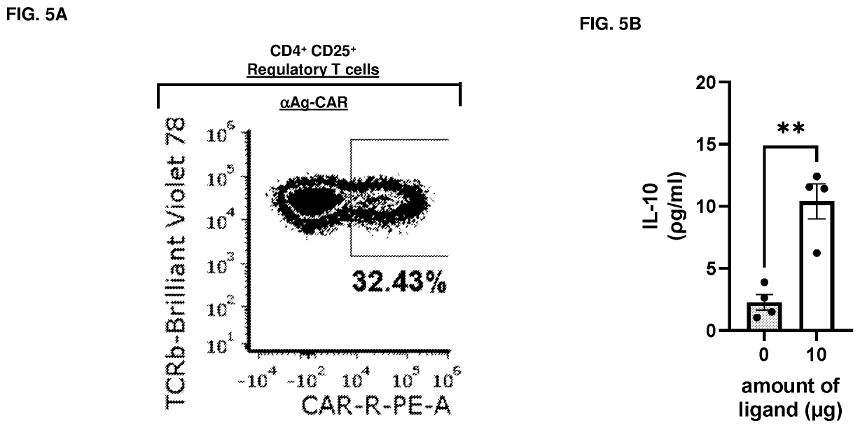

In result, we observed that CD4⁺CD25⁺ Tregs expressed αAg-CAR after lentiviral transduction, as is evident from the presence of the FC contour (32.43%) in the top right quadrant of FIG. 5A. Here, TCRb Brilliant Violet 78 concentration is used to identify T cells and the proportion of CAR expressing cells was determined by gating on cells with surface-bound CAR ligand (CAR-R-PE-A). Plots are gated on live, CD4⁺ T cells.

We also observed that the level of IL-10 produced by transduced CD4⁺CD25+ Tregs measured via ELISA (FIG. 5B) increased with higher ligand concentration (0 μg versus 10 μg), suggesting that the level of IL-10 secretion is a result of CAR function. CAR expression leads to ligand binding and produces more IL-10. Here, IL-10 (pg/ml) concentrations are plotted against the amount of ligand (pg). Bars represent mean+/−SEM with individual data points shown (n=4). Statistical significance assessed by unpaired T test: **p=0.0019.

Taken together, these data demonstrate that transduction of primary murine Treg cells to express an antigen-specific CAR resulted in transduced Treg cells that express a functional CAR construct.

### Example 6. Generating Regulatory T Cells with Preferential FoxP3 Promoter-Directed Transgene Expression within Reconstituted Immune Compartments Via Transplantation of Transduced Murine Bone Marrow HSC

### Objective

The objective of this study was to generate regulatory T cells with preferential FoxP3 promoter-directed transgene expression within reconstituted immune compartments via transplantation of transduced murine bone marrow hematopoietic stem cells (HSC).

### Materials and Methods

Lineage− bone marrow (Lineage− BM) cells were isolated and transduced with lentiviral constructs designed to express green fluorescent protein (GFP) under the control of a Treg (Foxp3) promoter. 10 weeks after transplantation, expression of GFP was assessed within the reconstituted immune compartment. A lentiviral construct was designed to contain conserved non-coding sequence (CNS) domains 1, 2 and 3 (CNS1, CNS2 and CNS3); a Foxp3 promoter; a coding sequence for green fluorescent protein (GFP) and 3′UTR sequence elements. The construct was designed to enhance transgene expression within the Treg compartment, while limiting transgene expression within other immune subsets. Following that, FC was performed in CD4⁺ CD25⁺ regulatory T cells derived from the spleen of transplanted animals to measure GFP expression. FC was again performed to measure the activity of the Foxp3-promoter in immune cells.

### Results

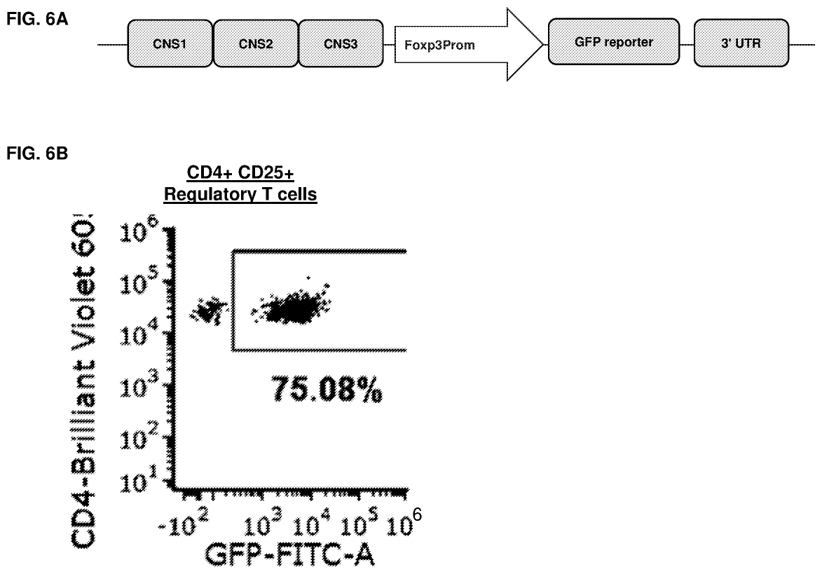

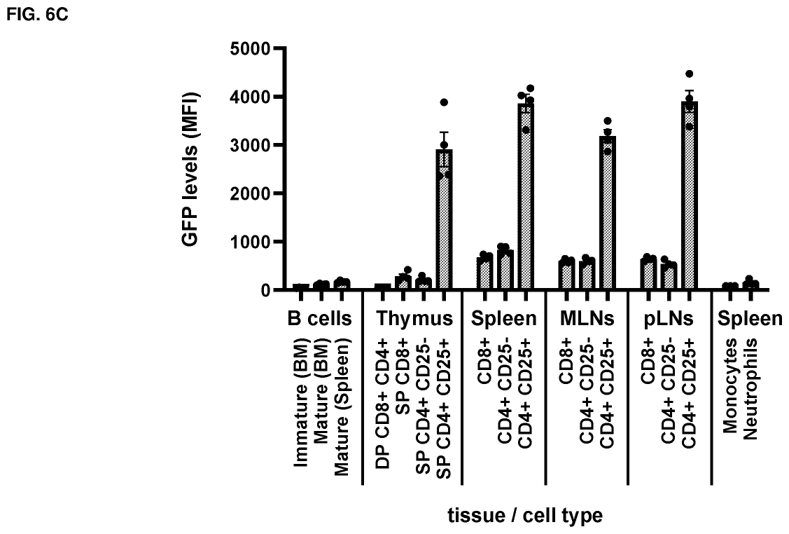

In result, we obtained a lentiviral construct (FIG. 6A) containing conserved non-coding sequence (CNS) domains 1, 2 and 3 (CNS1, CNS2 and CNS3); a Foxp3 promoter; a coding sequence for green fluorescent protein (GFP) and 3′UTR sequence elements, such that the lentiviral construct could express green fluorescent protein (GFP) under the control of a Treg (Foxp3) promoter.

We observed GFP expression in CD4⁺ CD25⁺ regulatory T cells derived from the spleen of transplanted animals, as evident from the presence of the FC contour (75.08%) in the top right region of FIG. 6B. Here, detection of CD4-Brilliant Violet 60 marker is used to identify T cells and the proportion of GFP expressing cells is determined by gating on GFP (GFP-FITC-A).

We also observed that Foxp3 promoter activity measured as GFP levels (MFI) varied based on the type of immune cell and tissue type (B cells, T cells, monocytes and neutrophils in thymus, spleen, MLNs (mesenteric lymph nodes) and pLNs (peripheral lymph nodes)) (FIG. 6C). Highest GFP levels are observed in CD4⁺ CD25⁺ regulatory T cells in thymus, spleen, MLNs and PLNs. Here, GFP levels (MFI) is plotted for each type of the immune cell in a particular tissue/cell type. Individual data points represent biological replicates with bars representing mean+/−SEM (n=4). Figure abbreviations: BM (bone marrow); DP (double positive); SP (single positive).

Taken together, these data demonstrate that transplantation of transduced murine bone marrow HSCs leads to the preferential expression of GFP in Treg cells when expression is driven by a FoxP3 promoter.

### Example 7. Generating CAR Expressing Regulatory T Cells In Vivo after Transplantation of Transduced Murine Bone Marrow Hematopoietic Stem Cells (HSC)

### Objective

The objective of this study was to generate CAR expressing regulatory T cells in vivo after transplantation of transduced murine bone marrow hematopoietic stem cells (HSC).

### Materials and Methods

Lineage− bone marrow (Lineage− BM) cells were isolated and transduced with lentiviral constructs to express an antigen-specific CAR (CAR+) or an irrelevant transgene (CAR−) under the control of a Treg (Foxp3) promoter. 10 weeks after transplantation, CAR expression was assessed throughout the immune compartment.

A lentiviral construct was designed to contain conserved non-coding sequence (CNS) domains 1, 2 and 3 (CNS1, CNS2 and CNS3); a Foxp3 promoter; a coding sequence for antigen-specific CAR (αAg-CAR) or an irrelevant transgene (CAR−) and 3′UTR sequence elements. Following that, FC was performed in CD4+CD25+ regulatory T cells derived from the spleen of transplanted animals to measure CAR expression.

FC was performed ex vivo to measure changes in Treg development and function in bone marrow chimeric mice. Total number of regulatory cells per spleen, expression levels of key regulatory T cell genes including the transcription factor, Foxp3 and surface marker CD25 were measured.

### Results

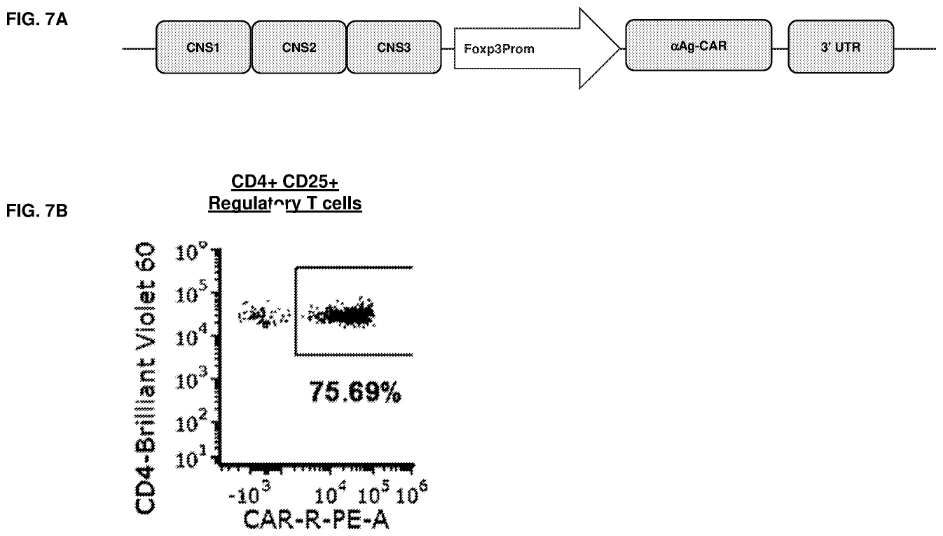

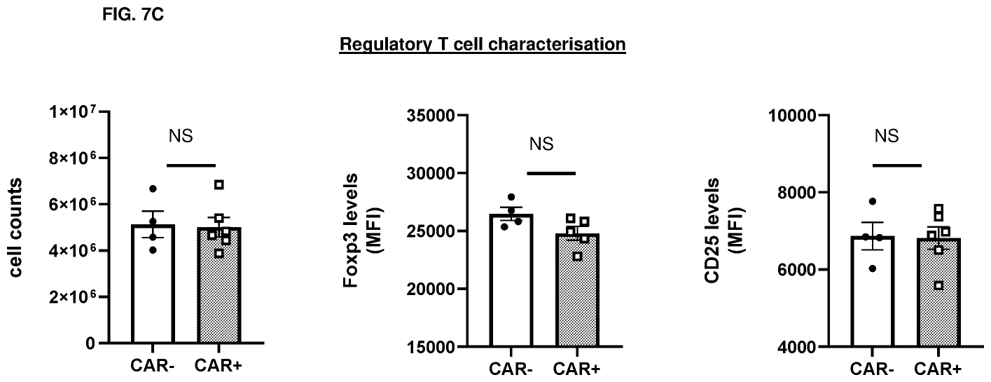

In result, we obtained a lentiviral construct (FIG. 7A) containing conserved non-coding sequence (CNS) domains 1, 2 and 3 (CNS1, CNS2 and CNS3); a Foxp3 promoter; a coding region for antigen-specific CAR (αAg-CAR) or an irrelevant transgene (CAR−) and 3′UTR sequence elements, such that the lentiviral construct could express an antigen-specific CAR (CAR+) or an irrelevant transgene (CAR−) under the control of a Treg (Foxp3) promoter.

We observed CAR expression in CD4⁺ CD25⁺ regulatory T cells derived from the spleen of transplanted animals (FIG. 7B), as evident from the presence of the FC contour (75.69%) in the top right quadrant where the bound CAR ligand concentration is high. Here, detection of CD4-Brilliant Violet 60 marker is used to identify T cells and the proportion of CAR expressing cells was determined by gating on cells with surface-bound CAR ligand (CAR-R-PE-A).

Additionally, we compared the number and phenotype of splenic regulatory T cells expressing a CAR (CAR+) or irrelevant transgene (CAR−) as assessed by ex vivo FC analysis (FIG. 7C). Comparable number and phenotype of splenic regulatory T cells expressing a CAR (CAR+) or irrelevant transgene (CAR−) were detected. Total number of regulatory cells per spleen were quantified and no significant difference was observed between CAR+ and CAR− group (left graph). Here cell counts are plotted according to CAR transgene expression. Expression levels of key regulatory T cell genes including the transcription factor Foxp3 and surface marker CD25 are quantified as MFI (middle and right graphs) respectively but no significant differences were found between the CAR+ and CAR− group in both cases. Here Foxp3 levels (MFI) and CD25 levels (MFI) are plotted according to CAR transgene expression. Individual data points represent biological replicates with bars representing mean+/−SEM (CAR− n=4; CAR+n=6). Statistical differences were assessed by unpaired T cell test with no significant differences detected.

### Example 8. Generating CAR Expressing Regulatory T Cells after Transduction of Murine Bone Marrow Hematopoietic Stem Cells (HSC) with Lentiviral Constructs Expressing an Antigen-Specific CAR (CAR+) or Irrelevant Transgene (CAR−) Under the Control of a Treg (Foxp3) Promoter, Followed by an Assessment of their Immunosuppressive Potential

### Objective

The objective of this study was to generate CAR expressing regulatory T cells after transduction of murine bone marrow hematopoietic stem cells (HSC) with lentiviral constructs expressing an antigen-specific CAR (CAR+) or irrelevant transgene (CAR−) under the control of a Treg (Foxp3) promoter, followed by an assessment of their immunosuppressive capacity using a cell tracer violet proliferation assay.

### Materials and Methods

Lineage-bone marrow (Lineage− BM) cells were isolated and transduced with lentiviral constructs to express an antigen-specific CAR (CAR+) or an irrelevant transgene (CAR−) under the control of a Treg (Foxp3) promoter. 10 weeks after transplantation, regulatory T cells were isolated from peripheral immune organs and assessed in vitro for changes in immune function. CAR expressing Tregs were assessed for immunosuppressive capacity by culturing Tregs with cell tracer violet labelled effector T cells. Effector T cells were stimulated with CD3/CD28 microbeads for 96 hrs in the presence of control CAR− or Ag-CAR+ Tregs. Proliferative responses were measured by dilution of Cell Tracer dye.

### Results

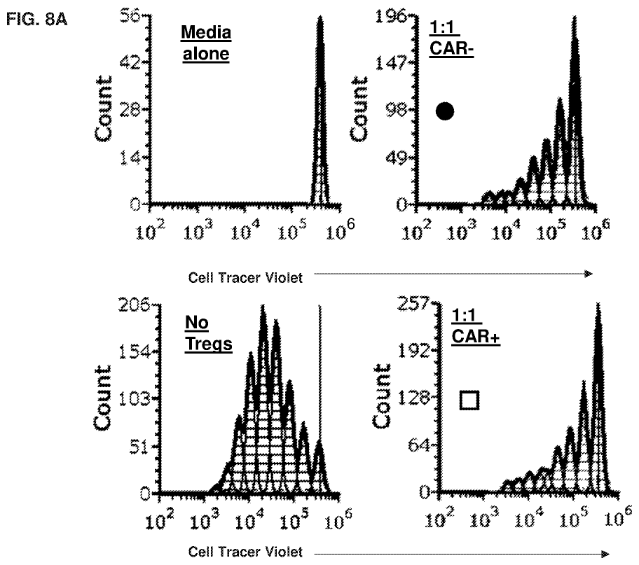

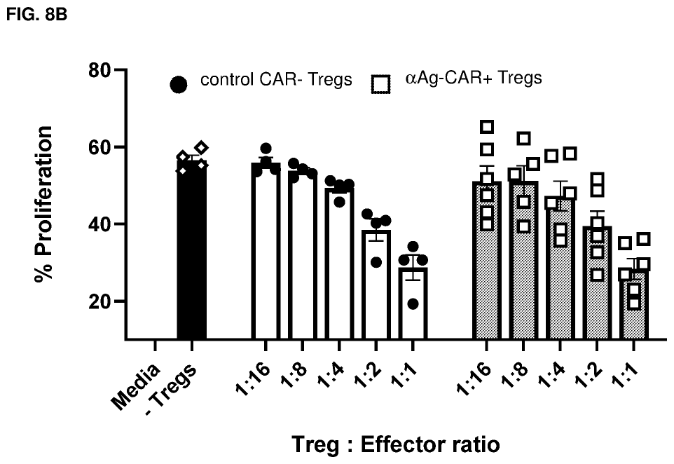

In result, we observed that both CAR− (FIG. 8A, top right) and Ag-CAR+ Tregs (FIG. 8A,bottom right) had immunosuppressive capacities and were able to reduce cell tracer violet labelled effector T cell proliferation, shown by the number of peaks within the histogram profiles, where each peak represents a cell division. Representative histograms depict cell tracer dye profiles for experimental conditions indicated.

We also observed that as the concentration of Treg to effector T cells increased, the % proliferative capacity of effector T cells decreased (FIG. 8B). Proliferative responses were quantified by dilution of Cell Tracer dye. Data represented as percentage of cell tracer labelled cells that have undergone division, “% Proliferation”. Individual data points represent biological replicates with bars representing mean+/−SEM (CAR− n=4; CAR+n=6). Statistical significance was assessed by paired T test for each comparable ratio of cells with no significant differences detected.

### Example 9. Generating CAR Expressing Regulatory T Cells after Transduction of Murine Bone Marrow Hematopoietic Stem Cells (HSC) with Lentiviral Constructs Expressing an Antigen-Specific CAR (CAR+) Under the Control of a Treg (Foxp3) Promoter or Irrelevant Transgene (CAR−), Followed by an Assessment of their Antigen-Specific Immunosuppressive Potential

### Objective

The objective of this study was to generate CAR expressing regulatory T cells after transduction of murine bone marrow hematopoietic stem cells (HSC) with lentiviral constructs expressing an antigen-specific CAR (CAR+) under the control of a Treg (Foxp3) promoter or irrelevant transgene (CAR−), followed by an assessment of the antigen-specific immunosuppressive function conferred by the CAR.

### Materials and Methods

Lineage− bone marrow (Lineage− BM) cells were isolated and transduced with lentiviral constructs to express an antigen-specific CAR (CAR+) under the control of a Treg specific (Foxp3) promoter or an irrelevant transgene (control CAR−). 10 weeks after transplantation, regulatory T cells were isolated from peripheral immune organs and cultured in vitro with CAR ligand for 48 hrs to assess activation. CD25 expression levels were measured via FC following stimulation with 10 μg CAR ligand in both control CAR− Tregs and αAg-CAR+ Tregs using a CD25-PE-Cy7-A ligand. Following that, both control CAR− Tregs and αAg-CAR+ Tregs were exposed to 10 μg CAR ligand in the absence or presence of CD3/CD28 microbeads for 48 hrs. Supernatants were collected and IL-10 secretion was determined by ELISA.

### Results

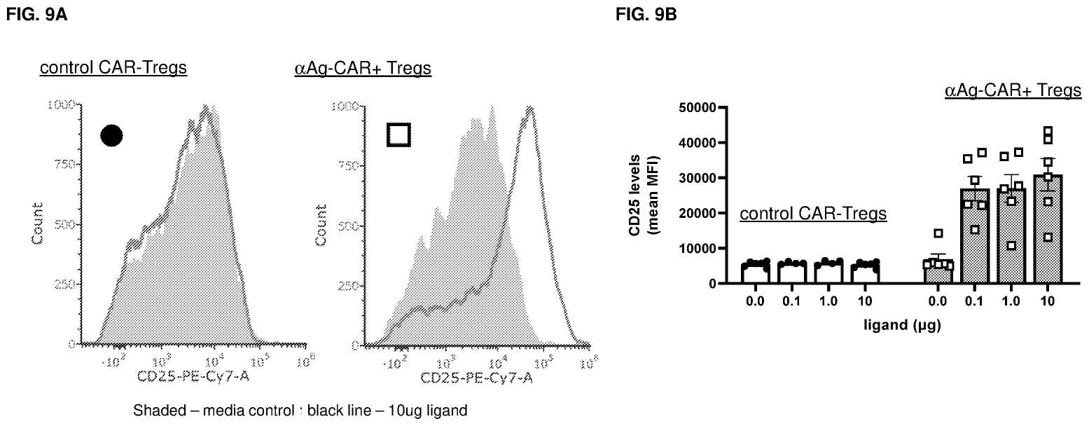

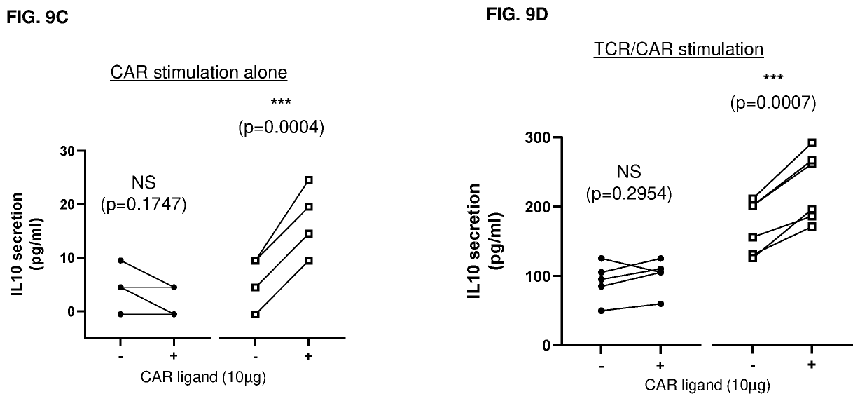

In result, we observed that, when stimulated with the corresponding ligand, αAg-CAR+ Tregs (right) had a much higher expression of CD25 compared to control CAR− Tregs (FIG. 9A). Here, the number of cells (counts) expressing different levels of CD25, determined by surface-bound CD25-PE-Cy7-A are shown. CD25 levels are quantified in control CAR− and CAR+ expressing regulatory T cells as mean MFI (FIG. 9B).

We also observed that the αAg-CAR+ Tregs (squares) secreted more IL-10 (pg/ml) compared to control CAR− Tregs (circles) after exposure to 10 μg CAR ligand in the absence (FIG. 9C) or presence of CD3/CD28 microbeads (FIG. 9D) for 48 hrs. αAg-CAR+ Tregs (squares) secreted more IL-10 (pg/ml) after exposure to 10 μg CAR ligand but there wasn't a significant difference in the amount of IL-10 secreted by control CAR+ Tregs before and after exposure to ligand. Statistical significance assessed by paired T test with p values shown.

### Example 10. Generation of a Pluripotent Cells Expressing an Autoantigen Binding Protein for the Treatment of Autoimmune Diseases

An exemplary method for making pluripotent cells, such as pluripotent hematopoietic cells (e.g., hematopoietic stem cells (HSCs), hematopoietic progenitor cells (HPCs), embryonic stem cells (ESCs), induced pluripotent stem cells (iPSCs), lymphoid progenitor cells, or CD34+ cells), that express an autoantigen-binding protein is by way of transduction. Retroviral vectors (e.g., a lentiviral vector, alpharetroviral vector, or gammaretroviral vector) containing, e.g., a suitable promoter, such as a Foxp3 promoter described herein, a suitable enhancer, such as a CNS1, CNS2, CNS3, and/or CNS0 enhancer described herein, and a nucleic acid cassette encoding an autoantigen binding protein can be engineered using vector production techniques described herein or known in the art. After the retroviral vector is engineered, the retrovirus can be used to transduce pluripotent hematopoietic cells (e.g., HSCs, HPCs, ESCs, iPSCs, lymphoid progenitor cells, or CD34+ cells) to generate a population of pluripotent hematopoietic cells that express the autoantigen binding protein.

Additional exemplary methods for making pluripotent hematopoietic cells that express an autoantigen-binding protein are transfection techniques. Using molecular biology procedures described herein and known in the art, plasmid DNA containing, for example, a promoter, one or more enhancers, and an autoantigen binding-protein can be produced. For example, a nucleic acid encoding an autoantigen binding-protein may be amplified from a human cell line using PCR-based techniques known in the art, or a nucleic acid encoding an autoantigen binding-protein may be synthesized, for example, using solid-phase polynucleotide synthesis procedures. The nucleic acid, promoter, and enhancer(s) can then be ligated into a plasmid of interest, for example, using suitable restriction endonuclease-mediated cleavage and ligation protocols. After the plasmid DNA is engineered, the plasmid can be used to transfect the pluripotent hematopoietic cells (e.g., HSCs, HPCs, ESCs, iPSCs, lymphoid progenitor cells, or CD34+ cells) using, for example, electroporation or another transfection technique described herein to generate a population of pluripotent hematopoietic cells that express the encoded protein(s).

### Example 11. Administration of a Therapeutic Composition to a Patient Suffering from an Autoimmune Disease

According to the methods disclosed herein, a patient, such as a human patient, can be treated so as to reduce or alleviate symptoms of an autoimmune disease and/or so as to target an underlying biochemical etiology of the disease. To this end, the patient may be administered, for example, a population of pluripotent cells, such as e.g., pluripotent hematopoietic cells (e.g., HSCs, HPCs, ESCs, iPSCs, lymphoid progenitor cells, or CD34+ cells), expressing an autoantigen binding protein under the control of lineage-specific transcription regulatory elements that are active in CD4+CD25+ regulatory T (Treg) cells. The population of pluripotent hematopoietic cells may be administered to the patient, for example, systemically (e.g., intravenously). The cells may be administered in a therapeutically effective amount, such as from 1×10⁶ cells/kg to 1×10¹² cells/kg or more (e.g., 1×10⁷ cells/kg, 1×10⁸ cells/kg, 1 ×10⁹ cells/kg, 1×10¹⁰ cells/kg, 1×10¹¹ cells/kg, 1×10¹² cells/kg, or more).

Before the population of cells is administered to the patient, one or more agents may be administered to the patient to ablate the patient's endogenous hematopoietic cell population, for example, by administration of a conditioning agent described herein.

The success of the treatment may be monitored by way of various clinical indicators. Effective treatment of an autoimmune disease using a composition of the disclosure may manifest, for example, as (i) sustained disease remission, such as sustained disease remission for at least one year; (ii) an observation of reduced inflammation or alleviation of pain in the patient; and/or (iii) an observation of reduced tissue damage in the patient.

### Exemplary Embodiments of the Invention

Exemplary embodiments of the invention are described in the enumerated paragraphs below.

1. A method of treating or preventing an autoimmune disease in a patient in need thereof, the method including the step of administering to the patient a population of pluripotent hematopoietic cells that include a nucleic acid cassette that encodes an autoantigen-binding protein, wherein the nucleic acid cassette is operably linked to one or more lineage-specific transcription regulatory elements that are active in CD4+CD25+ regulatory T (Treg) cells (i.e., specifically active in cells of the Treg lineage and not active in other cell types (e.g., other hematopoietic cells)).

2. A method of suppressing activity and/or proliferation of a population of autoreactive effector immune cells in a patient diagnosed as having an autoimmune disease, the method including the step of administering to the patient a population of pluripotent hematopoietic cells that include a nucleic acid cassette that encodes an autoantigen-binding protein, wherein the nucleic acid cassette is operably linked to one or more lineage-specific transcription regulatory elements that are active in CD4+CD25+ Treg cells (i.e., specifically active in cells of the Treg lineage and not active in other cell types (e.g., other hematopoietic cells)).

3. A method of inducing apoptosis of an autoreactive effector immune cell in a patient diagnosed as having an autoimmune disease, the method including the step of administering to the patient a population of pluripotent hematopoietic cells that include a nucleic acid cassette that encodes an autoantigen-binding protein, wherein the nucleic acid cassette is operably linked to one or more lineage-specific transcription regulatory elements that are active in CD4+CD25+ Treg cells (i.e., specifically active in cells of the Treg lineage and not active in other cell types (e.g., other hematopoietic cells)).

4. A method of protecting endogenous tissue from an autoimmune response in a patient diagnosed as having an autoimmune disease, the method including the step of administering to the patient a population of pluripotent hematopoietic cells that include a nucleic acid cassette that encodes an autoantigen-binding protein, wherein the nucleic acid cassette is operably linked to one or more lineage-specific transcription regulatory elements that are active in CD4+CD25+ Treg cells (i.e., specifically active in cells of the Treg lineage and not active in other cell types (e.g., other hematopoietic cells)).

5. A method of reducing inflammation in a patient diagnosed as having an autoimmune disease, the method including the step of administering to the patient a population of pluripotent hematopoietic cells that include a nucleic acid cassette that encodes an autoantigen-binding protein, wherein the nucleic acid cassette is operably linked to one or more lineage-specific transcription regulatory elements that are active in CD4+CD25+ Treg cells (i.e., specifically active in cells of the Treg lineage and not active in other cell types (e.g., other hematopoietic cells)).

6. The method of any one of embodiments 1-5, wherein the pluripotent hematopoietic cells are hematopoietic stem cells (HSCs) or hematopoietic progenitor cells (HPCs).

7. The method of any one of embodiments 1-5, wherein the pluripotent hematopoietic cells are embryonic stem cells.

8. The method of any one of embodiments 1-5, wherein the pluripotent hematopoietic cells are induced pluripotent stem cells.

9. The method of any one of embodiments 1-5, wherein the pluripotent hematopoietic cells are lymphoid progenitor cells.

10. The method of any one of embodiments 1-9, wherein the pluripotent hematopoietic cells are CD34+ cells.

11. The method of any one of embodiments 1-10, wherein the population of pluripotent hematopoietic cells is administered systemically to the patient.

12. The method of embodiment 11, wherein the population of pluripotent hematopoietic cells is administered to the patient by way of intravenous injection.

13. The method of any one of embodiments 1-12, wherein the pluripotent hematopoietic cells are autologous with respect to the patient.

14. The method of any one of embodiments 1-12, wherein the pluripotent hematopoietic cells are allogeneic with respect to the patient.

15. The method of embodiment 14, wherein the pluripotent hematopoietic cells are HLA-matched to the patient.

16. The method of any one of embodiments 1-15, wherein the pluripotent hematopoietic cells are transduced ex vivo with a viral vector that includes the nucleic acid cassette that encodes the autoantigen-binding protein.

17. The method of embodiment 16, wherein the viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

18. The method of embodiment 17, wherein the viral vector is a Retroviridae family viral vector.

19. The method of embodiment 18, wherein the Retroviridae family viral vector is a lentiviral vector.

20. The method of embodiment 18, wherein the Retroviridae family viral vector is an alpharetroviral vector or a gammaretroviral vector.

21. The method of any one of embodiments 17-20, wherein the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

22. The method of any one of embodiments 17-21, wherein the viral vector is a pseudotyped viral vector.

23. The method of embodiment 22, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

24. The method of embodiment 23, wherein the pseudotyped viral vector is a pseudotyped lentiviral vector.

25. The method of any one of embodiments 22-24, wherein the pseudotyped viral vector includes an envelope protein from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

26. The method of embodiment 25, wherein the pseudotyped viral vector includes a VSV-G envelope protein.

27. The method of any one of embodiments 1-15, wherein the pluripotent hematopoietic cells are transfected ex vivo with a polynucleotide that includes the nucleic acid cassette that encodes the autoantigen-binding protein.

28. The method of embodiment 27, wherein the pluripotent hematopoietic cells are transfected using a cationic polymer, diethylaminoethyldextran, polyethylenimine, a cationic lipid, a liposome, calcium phosphate, an activated dendrimer, and/or a magnetic bead.

29. The method of embodiment 27 or 28, wherein the pluripotent hematopoietic cells are transfected by way of electroporation, Nucleofection, squeeze-poration, sonoporation, optical transfection, Magnetofection, and/or impalefection.

30. The method of any one of embodiments 1-15, wherein the nucleic acid cassette is part of a transposable element.

31. The method of embodiment 30, wherein the nucleic acid cassette includes a transposase recognition and cleavage element for incorporation into a deoxyribonucleic acid (DNA) molecule of the pluripotent hematopoietic cell.

32. The method of embodiment 31, wherein the DNA molecule is a nuclear or mitochondrial DNA molecule and the transposase recognition and cleavage element promotes incorporation into the nuclear or mitochondrial DNA molecule.

33. The method of any one of embodiments 1-15, wherein the pluripotent hematopoietic cells are obtained by delivering to the cells a nuclease that catalyzes a single-strand break or a double-strand break at a target position within the genome of the cell.

34. The method of embodiment 33, wherein the nuclease is delivered to the cells in combination with a guide RNA (gRNA) that hybridizes to the target position within the genome of the cell.

35. The method of embodiment 33 or 34, wherein the nuclease is a clustered regularly interspaced short palindromic repeats (CRISPR)-associated protein.

36. The method of embodiment 35, wherein the CRISPR-associated protein is CRISPR-associated protein 9 (Cas9) or CRISPR-associated protein 12a (Cas12a).

37. The method of embodiment 33 or 34, wherein the nuclease is a transcription activator-like effector nuclease, a meganuclease, or a zinc finger nuclease.

38. The method of any one of embodiments 33-37, wherein while the cells are contacted with the nuclease, the cells are additionally contacted with a template polynucleotide that includes the nucleic acid cassette that encodes the autoantigen-binding protein.

39. The method of embodiment 38, wherein the template polynucleotide that includes a 5′ homology arm and a 3′ homology arm having nucleic acid sequences that are sufficiently similar to the nucleic acid sequences located 5′ to the target position and 3′ to the target position, respectively, to promote homologous recombination.

40. The method of embodiment 38 or 39, wherein the nuclease, gRNA, and/or template polynucleotide are delivered to the cells by contacting the cells with a viral vector that encodes the nuclease, gRNA, and/or template polynucleotide.

41. The method of embodiment 40, wherein the viral vector that encodes the nuclease, gRNA, and/or template polynucleotide is an AAV, an adenovirus, a parvovirus, a coronavirus, a rhabdovirus, a paramyxovirus, a picornavirus, an alphavirus, a herpes virus, a poxvirus, or a Retroviridae family virus.

42. The method of embodiment 41, wherein the viral vector that encodes the nuclease, gRNA, and/or template polynucleotide is a Retroviridae family virus.

43. The method of embodiment 42, wherein the Retroviridae family virus is a lentiviral vector, alpharetroviral vector, or gammaretroviral vector.

44. The method of embodiment 42 or 43, wherein the Retroviridae family virus that encodes the nuclease, gRNA, and/or template polynucleotide that includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

45. The method of embodiment 40, wherein the viral vector that encodes the nuclease, gRNA, and/or template polynucleotide is an integration-deficient lentiviral vector.

46. The method of embodiment 40, wherein the viral vector that encodes the nuclease, gRNA, and/or template polynucleotide is an AAV selected from the group consisting of AAV1, AAV2, AAV3, AAV4, AAV5, AAV6, AAV7, AAV8, AAV9, AAV10, and AAVrh74.

47. The method of any one of embodiments 1-46, wherein the one or more lineage-specific transcription regulatory elements include a Foxp3 promoter.

48. The method of embodiment 47, wherein the Foxp3 promoter has a nucleic acid sequence that is at least 85% identical to the nucleic acid sequence of SEQ ID NO: 1.

49. The method of embodiment 48, wherein the Foxp3 promoter has a nucleic acid sequence that is at least 90% identical to the nucleic acid sequence of SEQ ID NO: 1.

50. The method of embodiment 49, wherein the Foxp3 promoter has a nucleic acid sequence that is at least 95% identical to the nucleic acid sequence of SEQ ID NO: 1, optionally wherein the Foxp3 promoter has a nucleic acid sequence that is at least 96% identical, 97% identical, 98% identical, 99% identical, or more, to the nucleic acid sequence of SEQ ID NO: 1.

51. The method of embodiment 50, wherein the Foxp3 promoter has the nucleic acid sequence of SEQ ID NO: 1.

52. The method of embodiment 47, wherein the Foxp3 promoter has a nucleic acid sequence that is at least 85% identical to the nucleic acid sequence of SEQ ID NO: 2.

53. The method of embodiment 52, wherein the Foxp3 promoter has a nucleic acid sequence that is at least 90% identical to the nucleic acid sequence of SEQ ID NO: 2.

54. The method of embodiment 53, wherein the Foxp3 promoter has a nucleic acid sequence that is at least 95% identical to the nucleic acid sequence of SEQ ID NO: 2, optionally wherein the Foxp3 promoter has a nucleic acid sequence that is at least 96% identical, 97% identical, 98% identical, 99% identical, or more, to the nucleic acid sequence of SEQ ID NO: 2.

55. The method of embodiment 54, wherein the Foxp3 promoter has the nucleic acid sequence of SEQ ID NO: 2.

56. The method of embodiment 47, wherein the Foxp3 promoter has a nucleic acid sequence that is at least 85% identical to the nucleic acid sequence of SEQ ID NO: 3.

57. The method of embodiment 56, wherein the Foxp3 promoter has a nucleic acid sequence that is at least 90% identical to the nucleic acid sequence of SEQ ID NO: 3.

58. The method of embodiment 57, wherein the Foxp3 promoter has a nucleic acid sequence that is at least 95% identical to the nucleic acid sequence of SEQ ID NO: 3, optionally wherein the Foxp3 promoter has a nucleic acid sequence that is at least 96% identical, 97% identical, 98% identical, 99% identical, or more, to the nucleic acid sequence of SEQ ID NO: 3.

59. The method of embodiment 58, wherein the Foxp3 promoter has the nucleic acid sequence of SEQ ID NO: 3.

60. The method of embodiment 47, wherein the Foxp3 promoter has a nucleic acid sequence that is at least 85% identical to the nucleic acid sequence of SEQ ID NO: 4.

61. The method of embodiment 60, wherein the Foxp3 promoter has a nucleic acid sequence that is at least 90% identical to the nucleic acid sequence of SEQ ID NO: 4.

62. The method of embodiment 61, wherein the Foxp3 promoter has a nucleic acid sequence that is at least 95% identical to the nucleic acid sequence of SEQ ID NO: 4, optionally wherein the Foxp3 promoter has a nucleic acid sequence that is at least 96% identical, 97% identical, 98% identical, 99% identical, or more, to the nucleic acid sequence of SEQ ID NO: 4.

63. The method of embodiment 62, wherein the Foxp3 promoter has the nucleic acid sequence of SEQ ID NO: 4.

64. The method of any one of embodiments 47-63, wherein the Foxp3 promoter specifically binds transcription factor Nr4a and/or Foxo.

65. The method of any one of embodiments 1-64, wherein the one or more lineage-specific transcription regulatory elements include a CNS1 enhancer.

66. The method of embodiment 65, wherein the CNS1 enhancer has a nucleic acid sequence that is at least 85% identical to the nucleic acid sequence of SEQ ID NO: 5.

67. The method of embodiment 66, wherein the CNS1 enhancer has a nucleic acid sequence that is at least 90% identical to the nucleic acid sequence of SEQ ID NO: 5.

68. The method of embodiment 67, wherein the CNS1 enhancer has a nucleic acid sequence that is at least 95% identical to the nucleic acid sequence of SEQ ID NO: 5, optionally wherein the CNS1 enhancer has a nucleic acid sequence that is at least 96% identical, 97% identical, 98% identical, 99% identical, or more, to the nucleic acid sequence of SEQ ID NO: 5.

69. The method of embodiment 68, wherein the CNS1 enhancer has the nucleic acid sequence of SEQ ID NO: 5.

70. The method of embodiment 65, wherein the CNS1 enhancer has a nucleic acid sequence that is at least 85% identical to the nucleic acid sequence of SEQ ID NO: 6.

71. The method of embodiment 70, wherein the CNS1 enhancer has a nucleic acid sequence that is at least 90% identical to the nucleic acid sequence of SEQ ID NO: 6.

72. The method of embodiment 71, wherein the CNS1 enhancer has a nucleic acid sequence that is at least 95% identical to the nucleic acid sequence of SEQ ID NO: 6, optionally wherein the CNS1 enhancer has a nucleic acid sequence that is at least 96% identical, 97% identical, 98% identical, 99% identical, or more, to the nucleic acid sequence of SEQ ID NO: 6.

73. The method of embodiment 72, wherein the CNS1 enhancer has the nucleic acid sequence of SEQ ID NO: 6.

74. The method of embodiment 65, wherein the CNS1 enhancer has a nucleic acid sequence that is at least 85% identical to the nucleic acid sequence of SEQ ID NO: 7.

75. The method of embodiment 74, wherein the CNS1 enhancer has a nucleic acid sequence that is at least 90% identical to the nucleic acid sequence of SEQ ID NO: 7.

76. The method of embodiment 75, wherein the CNS1 enhancer has a nucleic acid sequence that is at least 95% identical to the nucleic acid sequence of SEQ ID NO: 7, optionally wherein the CNS1 enhancer has a nucleic acid sequence that is at least 96% identical, 97% identical, 98% identical, 99% identical, or more, to the nucleic acid sequence of SEQ ID NO: 7.

77. The method of embodiment 76, wherein the CNS1 enhancer has the nucleic acid sequence of SEQ ID NO: 7.

78. The method of embodiment 65, wherein the CNS1 enhancer has a nucleic acid sequence that is at least 85% identical to the nucleic acid sequence of SEQ ID NO: 8.

79. The method of embodiment 78, wherein the CNS1 enhancer has a nucleic acid sequence that is at least 90% identical to the nucleic acid sequence of SEQ ID NO: 8.

80. The method of embodiment 79, wherein the CNS1 enhancer has a nucleic acid sequence that is at least 95% identical to the nucleic acid sequence of SEQ ID NO: 8, optionally wherein the CNS1 enhancer has a nucleic acid sequence that is at least 96% identical, 97% identical, 98% identical, 99% identical, or more, to the nucleic acid sequence of SEQ ID NO: 8.

81. The method of embodiment 80, wherein the CNS1 enhancer has the nucleic acid sequence of SEQ ID NO: 8.

82. The method of any one of embodiments 65-81, wherein the CNS1 enhancer specifically binds transcription factor AP-1, NFAT, Smad3, and/or Foxo.

83. The method of any one of embodiments 1-82, wherein the one or more lineage-specific transcription regulatory elements include a CNS2 enhancer.

84. The method of embodiment 83, wherein the CNS2 enhancer has a nucleic acid sequence that is at least 85% identical to the nucleic acid sequence of SEQ ID NO: 9.

85. The method of embodiment 84, wherein the CNS2 enhancer has a nucleic acid sequence that is at least 90% identical to the nucleic acid sequence of SEQ ID NO: 9.

86. The method of embodiment 85, wherein the CNS2 enhancer has a nucleic acid sequence that is at least 95% identical to the nucleic acid sequence of SEQ ID NO: 9, optionally wherein the CNS2 enhancer has a nucleic acid sequence that is at least 96% identical, 97% identical, 98% identical, 99% identical, or more, to the nucleic acid sequence of SEQ ID NO: 9.

87. The method of embodiment 86, wherein the CNS2 enhancer has the nucleic acid sequence of SEQ ID NO: 9.

88. The method of embodiment 83, wherein the CNS2 enhancer has a nucleic acid sequence that is at least 85% identical to the nucleic acid sequence of SEQ ID NO: 10.

89. The method of embodiment 88, wherein the CNS2 enhancer has a nucleic acid sequence that is at least 90% identical to the nucleic acid sequence of SEQ ID NO: 10.

90. The method of embodiment 89, wherein the CNS2 enhancer has a nucleic acid sequence that is at least 95% identical to the nucleic acid sequence of SEQ ID NO: 10, optionally wherein the CNS2 enhancer has a nucleic acid sequence that is at least 96% identical, 97% identical, 98% identical, 99% identical, or more, to the nucleic acid sequence of SEQ ID NO: 10.

91. The method of embodiment 90, wherein the CNS2 enhancer has the nucleic acid sequence of SEQ ID NO: 10.

92. The method of embodiment 83, wherein the CNS2 enhancer has a nucleic acid sequence that is at least 85% identical to the nucleic acid sequence of SEQ ID NO: 11.

93. The method of embodiment 92, wherein the CNS2 enhancer has a nucleic acid sequence that is at least 90% identical to the nucleic acid sequence of SEQ ID NO: 11.

94. The method of embodiment 93, wherein the CNS2 enhancer has a nucleic acid sequence that is at least 95% identical to the nucleic acid sequence of SEQ ID NO: 11, optionally wherein the CNS2 enhancer has a nucleic acid sequence that is at least 96% identical, 97% identical, 98% identical, 99% identical, or more, to the nucleic acid sequence of SEQ ID NO: 11.

95. The method of embodiment 94, wherein the CNS2 enhancer has the nucleic acid sequence of SEQ ID NO: 11.

96. The method of embodiment 83, wherein the CNS2 enhancer has a nucleic acid sequence that is at least 85% identical to the nucleic acid sequence of SEQ ID NO: 12.

97. The method of embodiment 96, wherein the CNS2 enhancer has a nucleic acid sequence that is at least 90% identical to the nucleic acid sequence of SEQ ID NO: 12.

98. The method of embodiment 97, wherein the CNS2 enhancer has a nucleic acid sequence that is at least 95% identical to the nucleic acid sequence of SEQ ID NO: 12, optionally wherein the CNS2 enhancer has a nucleic acid sequence that is at least 96% identical, 97% identical, 98% identical, 99% identical, or more, to the nucleic acid sequence of SEQ ID NO: 12.

99. The method of embodiment 98, wherein the CNS2 enhancer has the nucleic acid sequence of SEQ ID NO: 12.

100. The method of any one of embodiments 83-99, wherein the CNS2 enhancer specifically binds transcription factor Runx, Foxp3, Ets-1, CREB, Stat5, NFAT, and/or c-Rel.

101. The method of any one of embodiments 1-100, wherein the one or more lineage-specific transcription regulatory elements include a CNS3 enhancer.

102. The method of embodiment 101, wherein the CNS3 enhancer has a nucleic acid sequence that is at least 85% identical to the nucleic acid sequence of SEQ ID NO: 13.

103. The method of embodiment 102, wherein the CNS3 enhancer has a nucleic acid sequence that is at least 90% identical to the nucleic acid sequence of SEQ ID NO: 13.

104. The method of embodiment 103, wherein the CNS3 enhancer has a nucleic acid sequence that is at least 95% identical to the nucleic acid sequence of SEQ ID NO: 13, optionally wherein the CNS3 enhancer has a nucleic acid sequence that is at least 96% identical, 97% identical, 98% identical, 99% identical, or more, to the nucleic acid sequence of SEQ ID NO: 13.

105. The method of embodiment 104, wherein the CNS3 enhancer has the nucleic acid sequence of SEQ ID NO: 13.

106. The method of embodiment 101, wherein the CNS3 enhancer has a nucleic acid sequence that is at least 85% identical to the nucleic acid sequence of SEQ ID NO: 14.

107. The method of embodiment 106, wherein the CNS3 enhancer has a nucleic acid sequence that is at least 90% identical to the nucleic acid sequence of SEQ ID NO: 14.

108. The method of embodiment 107, wherein the CNS3 enhancer has a nucleic acid sequence that is at least 95% identical to the nucleic acid sequence of SEQ ID NO: 14, optionally wherein the CNS3 enhancer has a nucleic acid sequence that is at least 96% identical, 97% identical, 98% identical, 99% identical, or more, to the nucleic acid sequence of SEQ ID NO: 14.

109. The method of embodiment 108, wherein the CNS3 enhancer has the nucleic acid sequence of SEQ ID NO: 14.

110. The method of embodiment 101, wherein the CNS3 enhancer has a nucleic acid sequence that is at least 85% identical to the nucleic acid sequence of SEQ ID NO: 15.

111. The method of embodiment 110, wherein the CNS3 enhancer has a nucleic acid sequence that is at least 90% identical to the nucleic acid sequence of SEQ ID NO: 15.

112. The method of embodiment 111, wherein the CNS3 enhancer has a nucleic acid sequence that is at least 95% identical to the nucleic acid sequence of SEQ ID NO: 15, optionally wherein the CNS3 enhancer has a nucleic acid sequence that is at least 96% identical, 97% identical, 98% identical, 99% identical, or more, to the nucleic acid sequence of SEQ ID NO: 15.

113. The method of embodiment 112, wherein the CNS3 enhancer has the nucleic acid sequence of SEQ ID NO: 15.

114. The method of embodiment 101, wherein the CNS3 enhancer has a nucleic acid sequence that is at least 85% identical to the nucleic acid sequence of SEQ ID NO: 16.

115. The method of embodiment 114, wherein the CNS3 enhancer has a nucleic acid sequence that is at least 90% identical to the nucleic acid sequence of SEQ ID NO: 16.

116. The method of embodiment 115, wherein the CNS3 enhancer has a nucleic acid sequence that is at least 95% identical to the nucleic acid sequence of SEQ ID NO: 16, optionally wherein the CNS3 enhancer has a nucleic acid sequence that is at least 96% identical, 97% identical, 98% identical, 99% identical, or more, to the nucleic acid sequence of SEQ ID NO: 16.

117. The method of embodiment 116, wherein the CNS3 enhancer has the nucleic acid sequence of SEQ ID NO: 16.

118. The method of any one of embodiments 101-117, wherein the CNS3 enhancer specifically binds transcription factor Foxo and/or c-Rel.

119. The method of any one of embodiments 1-118, wherein the one or more lineage-specific transcription regulatory elements include a CNS0 enhancer.

120. The method of embodiment 119, wherein the CNS0 enhancer has a nucleic acid sequence that is at least 85% identical to the nucleic acid sequence of SEQ ID NO: 17.

121. The method of embodiment 120, wherein the CNS0 enhancer has a nucleic acid sequence that is at least 90% identical to the nucleic acid sequence of SEQ ID NO: 17.

122. The method of embodiment 121, wherein the CNS0 enhancer has a nucleic acid sequence that is at least 95% identical to the nucleic acid sequence of SEQ ID NO: 17, optionally wherein the CNS0 enhancer has a nucleic acid sequence that is at least 96% identical, 97% identical, 98% identical, 99% identical, or more, to the nucleic acid sequence of SEQ ID NO: 17.

123. The method of embodiment 122, wherein the CNS0 enhancer has the nucleic acid sequence of SEQ ID NO: 17.

124. The method of embodiment 119, wherein the CNS0 enhancer has a nucleic acid sequence that is at least 85% identical to the nucleic acid sequence of SEQ ID NO: 18.

125. The method of embodiment 124, wherein the CNS0 enhancer has a nucleic acid sequence that is at least 90% identical to the nucleic acid sequence of SEQ ID NO: 18.

126. The method of embodiment 125, wherein the CNS0 enhancer has a nucleic acid sequence that is at least 95% identical to the nucleic acid sequence of SEQ ID NO: 18, optionally wherein the CNS0 enhancer has a nucleic acid sequence that is at least 96% identical, 97% identical, 98% identical, 99% identical, or more, to the nucleic acid sequence of SEQ ID NO: 18.

127. The method of embodiment 126, wherein the CNS0 enhancer has the nucleic acid sequence of SEQ ID NO: 18.

128. The method of embodiment 119, wherein the CNS0 enhancer has a nucleic acid sequence that is at least 85% identical to the nucleic acid sequence of SEQ ID NO: 19.

129. The method of embodiment 128, wherein the CNS0 enhancer has a nucleic acid sequence that is at least 90% identical to the nucleic acid sequence of SEQ ID NO: 19.

130. The method of embodiment 129, wherein the CNS0 enhancer has a nucleic acid sequence that is at least 95% identical to the nucleic acid sequence of SEQ ID NO: 19, optionally wherein the CNS0 enhancer has a nucleic acid sequence that is at least 96% identical, 97% identical, 98% identical, 99% identical, or more, to the nucleic acid sequence of SEQ ID NO: 19.

131. The method of embodiment 130, wherein the CNS0 enhancer has the nucleic acid sequence of SEQ ID NO: 19.

132. The method of embodiment 119, wherein the CNS0 enhancer has a nucleic acid sequence that is at least 85% identical to the nucleic acid sequence of SEQ ID NO: 20.

133. The method of embodiment 132, wherein the CNS0 enhancer has a nucleic acid sequence that is at least 90% identical to the nucleic acid sequence of SEQ ID NO: 20.

134. The method of embodiment 133, wherein the CNS0 enhancer has a nucleic acid sequence that is at least 95% identical to the nucleic acid sequence of SEQ ID NO: 20, optionally wherein the CNS0 enhancer has a nucleic acid sequence that is at least 96% identical, 97% identical, 98% identical, 99% identical, or more, to the nucleic acid sequence of SEQ ID NO: 20.

135. The method of embodiment 134, wherein the CNS0 enhancer has the nucleic acid sequence of SEQ ID NO: 20.

136. The method of any one of embodiments 119-135, wherein the CNS0 enhancer specifically binds transcription factor Satb1 and/or Stat5.

137. The method of any one of embodiments 1-136, wherein the nucleic acid cassette is operably linked to a riboswitch.

138. The method of embodiment 137, wherein binding of a ligand to the riboswitch induces expression of the nucleic acid cassette.

139. The method of any one of embodiments 1-138, wherein the autoantigen-binding protein is a single-chain polypeptide.

140. The method of any one of embodiments 1-139, wherein the autoantigen-binding protein is a chimeric antigen receptor (CAR).

141. The method of embodiment 140, wherein the chimeric antigen receptor includes an antigen recognition domain, a hinge domain, a transmembrane domain, and one or more intracellular signaling domains.

142. The method of embodiment 141, wherein the one or more intracellular signaling domains include one or more primary intracellular signaling domains and optionally one or more costimulatory intracellular signaling domains.

143. The method of embodiment 141 or 142, wherein the antigen recognition domain is a single-chain antibody fragment, optionally wherein the single-chain antibody fragment is a single-chain Fv molecule (scFv).

144. The method of any one of embodiments 141-143, wherein the hinge domain is a CD28, CD8, IgG1/IgG4, CD4, CD7, or IgD hinge domain.

145. The method of embodiment 144, wherein the hinge domain is a CD28 hinge domain.

146. The method of any one of embodiments 141-145, wherein the transmembrane domain includes a CD28, CD3 zeta, CD8, FcRIγ, CD4, CD7, OX40, or MHC (H2-Kb) transmembrane domain.

147. The method of embodiment 146, wherein the transmembrane domain includes a CD28 transmembrane domain.

148. The method of any one of embodiments 142-147, wherein the one or more primary intracellular signaling domains are selected from the group consisting of a CD3 zeta, FcR gamma, FcR beta, CD3 gamma, CD3 delta, CD3 epsilon, CD5, CD22, CD79a, CD79b, CD278 (ICOS), CD66d, DAP10, and a DAP12 intracellular signaling domain.

149. The method of embodiment 148, wherein at least one of the one or more primary intracellular signaling domains is a CD3 zeta intracellular signaling domain.

150. The method of any one of embodiments 142-149, wherein the one or more costimulatory intracellular signaling domains are selected from the group consisting of a CD27, CD28, 4-1 BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, CD83, CDS, ICAM-1, LFA-1 (CD11a/CD18), an MHC class I molecule, BTLA, and a Toll ligand receptor intracellular signaling domain.

151. The method of embodiment 150, wherein at least one of the one or more co-stimulatory intracellular signaling domains is a CD28 intracellular signaling domain.

152. The method of any one of embodiments 141-151, wherein the chimeric antigen receptor includes an N-terminal leader sequence.

153. The method of any one of embodiments 141-152, wherein the antigen recognition domain includes an N-terminal leader sequence.

154. The method of embodiment 153, wherein the N-terminal leader sequence of the antigen recognition domain is cleaved from the antigen recognition domain during cellular processing and localization of the chimeric antigen receptor to the cellular membrane.

155. The method of any one of embodiments 1-138, wherein the autoantigen-binding protein is a multi-chain protein.

156. The method of embodiment 155, wherein the autoantigen-binding protein is a full-length antibody, a dual-variable immunoglobulin domain, a diabody, a triabody, an antibody-like protein scaffold, a Fab fragment, or a F(ab′)₂ molecule.

157. The method of any one of embodiments 1-156, wherein the autoimmune disease is type 1 diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barré, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Ménière's Disease, Mixed Connective Tissue Disease, Multiple Sclerosis, Myasthenia Gravis, Neuromyelitis Optica, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, or Wegener's Granulomatosis.

158. The method of any one of embodiments 1-157, wherein the autoantigen is myelin oligodendrocyte glycoprotein, aquaporin 4, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double-stranded DNA, single-stranded DNA, transferrin, fetuin, factor II, factor VII, fibrin, fibrinogen, C1, C1q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

159. The method of embodiment 157, wherein the autoimmune disease is multiple sclerosis and the autoantigen is myelin oligodendrocyte glycoprotein.

160. The method of embodiment 157, wherein the autoimmune disease is type 1 diabetes and the autoantigen is insulin, GAD-65, IA-2, or ZnT8.

161. The method of embodiment 157, wherein the autoimmune disease is rheumatoid arthritis and the autoantigen is collagen II, the Fc portion of immunoglobin, citrullinated peptides, carbamylated peptides, or HSP65.

162. The method of embodiment 157, wherein the autoimmune disease is myasthenia gravis and the autoantigen is AChR, MuSK, or LRP4.

163. The method of embodiment 157, wherein the autoimmune disease is lupus and the autoantigen is histone II A.

164. The method of embodiment 157, wherein the autoimmune disease is hypothyroidism and the autoantigen is a protein expressed in the thyroid gland.

165. The method of embodiment 157, wherein the autoimmune disease is Graves' disease and the autoantigen is the thyrotrophin receptor.

166. The method of embodiment 157, wherein the autoimmune disease is pemphigus vulgaris and the autoantigen is double-stranded DNA.

167. The method of embodiment 157, wherein the autoimmune disease is psoriasis and the autoantigen is double-stranded DNA.

168. The method of embodiment 157, wherein the autoimmune disease is neuromyelitis optica and the autoantigen is aquaporin 4.

169. The method of any one of embodiments 1-168, wherein prior to administering the population of pluripotent hematopoietic cells to the patient, a population of precursor cells is isolated from the patient or a donor, and wherein the precursor cells are expanded and genetically modified ex vivo to yield the population of cells being administered to the patient.

170. The method of embodiment 169, wherein the precursor cells are CD34+ HSCs, and wherein the precursor cells are expanded without substantial loss of HSC functional potential.

171. The method of embodiment 169 or 170, wherein prior to isolation of the precursor cells from the patient or donor, the patient or donor is administered one or more pluripotent hematopoietic cell mobilization agents.

172. The method of any one of embodiments 1-171, wherein prior to administering the population of pluripotent hematopoietic cells to the patient, a population of endogenous pluripotent hematopoietic cells is ablated in the patient by administration of one or more conditioning agents to the patient.

173. The method of any one of embodiments 1-171, the method including the step of ablating a population of endogenous pluripotent hematopoietic cells in the patient by administering to the patient one or more conditioning agents prior to administering the population of pluripotent hematopoietic cells to the patient.

174. The method of embodiment 172 or 173, wherein the one or more conditioning agents are non-myeloablative conditioning agents.

175. The method of any one of embodiments 172-174, wherein the one or more conditioning agents deplete a population of CD34+ cells in the patient.

176. The method of embodiment 175, wherein the depleted CD34+ cells are lymphoid progenitor cells.

177. The method of any one of embodiments 172-176, wherein the one or more conditioning agents include an antibody or antigen-binding fragment thereof.

178. The method of embodiment 177, wherein the antibody or antigen-binding fragment thereof binds to CD117, HLA-DR, CD34, CD90, CD45, or CD133.

179. The method of embodiment 178, wherein the antibody or antigen-binding fragment thereof binds to CD117.

180. The method of any one of embodiments 177-179, wherein the antibody or antigen-binding fragment thereof is conjugated to a cytotoxin.

181. The method of any one of embodiments 1-180, wherein upon administration of the population of pluripotent hematopoietic cells to the patient, the administered cells, or progeny thereof, differentiate into CD4+CD25+ Treg cells.

182. The method of any one of embodiments 1-181, wherein the patient is a mammal and the cells are mammalian cells.

183. The method of embodiment 182, wherein the mammal is a human and the cells are human cells.

184. A pharmaceutical composition including (i) a population of pluripotent hematopoietic cells that include a nucleic acid cassette that encodes an autoantigen-binding protein, wherein the nucleic acid cassette is operably linked to one or more lineage-specific transcription regulatory elements that are active in CD4+CD25+ Treg cells (i.e., specifically active in cells of the Treg lineage and not active in other cell types (e.g., other hematopoietic cells)), and (ii) one or more pharmaceutically acceptable excipients, carriers, or diluents.

185. The pharmaceutical composition of embodiment 184, wherein the pluripotent hematopoietic cells are HSCs or HPCs.

186. The pharmaceutical composition of embodiment 184, wherein the pluripotent hematopoietic cells are embryonic stem cells.

187. The pharmaceutical composition of embodiment 184, wherein the pluripotent hematopoietic cells are induced pluripotent stem cells.

188. The pharmaceutical composition of embodiment 184, wherein the pluripotent hematopoietic cells are lymphoid progenitor cells.

189. The pharmaceutical composition of any one of embodiments 184-188, wherein the pluripotent hematopoietic cells are CD34+ cells.

190. The pharmaceutical composition of any one of embodiments 184-189, wherein the pluripotent hematopoietic cells are transduced ex vivo with a viral vector that includes the nucleic acid cassette that encodes the autoantigen-binding protein.

191. The pharmaceutical composition of embodiment 190, wherein the viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

192. The pharmaceutical composition of embodiment 191, wherein the viral vector is a Retroviridae family viral vector.

193. The pharmaceutical composition of embodiment 192, wherein the Retroviridae family viral vector is a lentiviral vector.

194. The pharmaceutical composition of embodiment 192, wherein the Retroviridae family viral vector is an alpharetroviral vector or a gammaretroviral vector.

195. The pharmaceutical composition of any one of embodiments 191-194, wherein the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

196. The pharmaceutical composition of any one of embodiments 191-195, wherein the viral vector is a pseudotyped viral vector.

197. The pharmaceutical composition of embodiment 196, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

198. The pharmaceutical composition of embodiment 197, wherein the pseudotyped viral vector is a pseudotyped lentiviral vector.

199. The pharmaceutical composition of any one of embodiments 196-198, wherein the pseudotyped viral vector includes an envelope protein from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

200. The pharmaceutical composition of embodiment 199, wherein the pseudotyped viral vector includes a VSV-G envelope protein.

201. The pharmaceutical composition of any one of embodiments 184-189, wherein the pluripotent hematopoietic cells are transfected ex vivo with a polynucleotide that includes the nucleic acid cassette that encodes the autoantigen-binding protein.

202. The pharmaceutical composition of embodiment 201, wherein the pluripotent hematopoietic cells are transfected using a cationic polymer, diethylaminoethyldextran, polyethylenimine, a cationic lipid, a liposome, calcium phosphate, an activated dendrimer, and/or a magnetic bead.

203. The pharmaceutical composition of embodiment 201 or 202, wherein the pluripotent hematopoietic cells are transfected by way of electroporation, Nucleofection, squeeze-poration, sonoporation, optical transfection, Magnetofection, and/or impalefection.

204. The pharmaceutical composition of any one of embodiments 184-189, wherein the nucleic acid cassette is part of a transposable element.

205. The pharmaceutical composition of embodiment 204, wherein the nucleic acid cassette includes a transposase recognition and cleavage element for incorporation into a DNA molecule of the pluripotent hematopoietic cell.

206. The pharmaceutical composition of embodiment 205, wherein the DNA molecule is a nuclear or mitochondrial DNA molecule and the transposase recognition and cleavage element promotes incorporation into the nuclear or mitochondrial DNA molecule.

207. The pharmaceutical composition of any one of embodiments 184-189, wherein the pluripotent hematopoietic cells are obtained by delivering to the cells a nuclease that catalyzes a single-strand break or a double-strand break at a target position within the genome of the cell.

208. The pharmaceutical composition of embodiment 207, wherein the nuclease is delivered to the cells in combination with a gRNA that hybridizes to the target position within the genome of the cell.

209. The pharmaceutical composition of embodiment 207 or 208, wherein the nuclease is a CRISPR-associated protein.

210. The pharmaceutical composition of embodiment 209, wherein the CRISPR-associated protein is Cas9 or Cas12a.

211. The pharmaceutical composition of embodiment 207 or 208, wherein the nuclease is a transcription activator-like effector nuclease, a meganuclease, or a zinc finger nuclease.

212. The pharmaceutical composition of any one of embodiments 207-211, wherein while the cells are contacted with the nuclease, the cells are additionally contacted with a template polynucleotide that includes the nucleic acid cassette that encodes the autoantigen-binding protein.

213. The pharmaceutical composition of embodiment 212, wherein the template polynucleotide includes a 5′ homology arm and a 3′ homology arm having nucleic acid sequences that are sufficiently similar to the nucleic acid sequences located 5′ to the target position and 3′ to the target position, respectively, to promote homologous recombination.

214. The pharmaceutical composition of embodiment 212 or 213, wherein the nuclease, gRNA, and/or template polynucleotide are delivered to the cells by contacting the cells with a viral vector that encodes the nuclease, gRNA, and/or template polynucleotide.

215. The pharmaceutical composition of embodiment 214, wherein the viral vector that encodes the nuclease, gRNA, and/or template polynucleotide is an AAV, an adenovirus, a parvovirus, a coronavirus, a rhabdovirus, a paramyxovirus, a picornavirus, an alphavirus, a herpes virus, a poxvirus, or a Retroviridae family virus.

216. The pharmaceutical composition of embodiment 215, wherein the viral vector that encodes the nuclease, gRNA, and/or template polynucleotide is a Retroviridae family virus.

217. The pharmaceutical composition of embodiment 216, wherein the Retroviridae family virus is a lentiviral vector, alpharetroviral vector, or gammaretroviral vector.

218. The pharmaceutical composition of embodiment 216 or 217, wherein the Retroviridae family virus that encodes the nuclease, gRNA, and/or template polynucleotide includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

219. The pharmaceutical composition of embodiment 214, wherein the viral vector that encodes the nuclease, gRNA, and/or template polynucleotide is an integration-deficient lentiviral vector.

220. The pharmaceutical composition of embodiment 214, wherein the viral vector that encodes the nuclease, gRNA, and/or template polynucleotide is an AAV selected from the group consisting of AAV1, AAV2, AAV3, AAV4, AAV5, AAV6, AAV7, AAV8, AAV9, AAV10, and AAVrh74.

221. The pharmaceutical composition of any one of embodiments 184-220, wherein the one or more lineage-specific transcription regulatory elements include a Foxp3 promoter.

222. The pharmaceutical composition of embodiment 221, wherein the Foxp3 promoter has a nucleic acid sequence that is at least 85% identical to the nucleic acid sequence of SEQ ID NO: 1.

223. The pharmaceutical composition of embodiment 222, wherein the Foxp3 promoter has a nucleic acid sequence that is at least 90% identical to the nucleic acid sequence of SEQ ID NO: 1.

224. The pharmaceutical composition of embodiment 223, wherein the Foxp3 promoter has a nucleic acid sequence that is at least 95% identical to the nucleic acid sequence of SEQ ID NO: 1, optionally wherein the Foxp3 promoter has a nucleic acid sequence that is at least 96% identical, 97% identical, 98% identical, 99% identical, or more, to the nucleic acid sequence of SEQ ID NO: 1.

225. The pharmaceutical composition of embodiment 224, wherein the Foxp3 promoter has the nucleic acid sequence of SEQ ID NO: 1.

226. The pharmaceutical composition of embodiment 221, wherein the Foxp3 promoter has a nucleic acid sequence that is at least 85% identical to the nucleic acid sequence of SEQ ID NO: 2.

227. The pharmaceutical composition of embodiment 226, wherein the Foxp3 promoter has a nucleic acid sequence that is at least 90% identical to the nucleic acid sequence of SEQ ID NO: 2.

228. The pharmaceutical composition of embodiment 227, wherein the Foxp3 promoter has a nucleic acid sequence that is at least 95% identical to the nucleic acid sequence of SEQ ID NO: 2, optionally wherein the Foxp3 promoter has a nucleic acid sequence that is at least 96% identical, 97% identical, 98% identical, 99% identical, or more, to the nucleic acid sequence of SEQ ID NO: 2.

229. The pharmaceutical composition of embodiment 228, wherein the Foxp3 promoter has the nucleic acid sequence of SEQ ID NO: 2.

230. The pharmaceutical composition of embodiment 221, wherein the Foxp3 promoter has a nucleic acid sequence that is at least 85% identical to the nucleic acid sequence of SEQ ID NO: 3.

231. The pharmaceutical composition of embodiment 230, wherein the Foxp3 promoter has a nucleic acid sequence that is at least 90% identical to the nucleic acid sequence of SEQ ID NO: 3.

232. The pharmaceutical composition of embodiment 231, wherein the Foxp3 promoter has a nucleic acid sequence that is at least 95% identical to the nucleic acid sequence of SEQ ID NO: 3, optionally wherein the Foxp3 promoter has a nucleic acid sequence that is at least 96% identical, 97% identical, 98% identical, 99% identical, or more, to the nucleic acid sequence of SEQ ID NO: 3.

233. The pharmaceutical composition of embodiment 232, wherein the Foxp3 promoter has the nucleic acid sequence of SEQ ID NO: 3.

234. The pharmaceutical composition of embodiment 221, wherein the Foxp3 promoter has a nucleic acid sequence that is at least 85% identical to the nucleic acid sequence of SEQ ID NO: 4.

235. The pharmaceutical composition of embodiment 234, wherein the Foxp3 promoter has a nucleic acid sequence that is at least 90% identical to the nucleic acid sequence of SEQ ID NO: 4.

236. The pharmaceutical composition of embodiment 235, wherein the Foxp3 promoter has a nucleic acid sequence that is at least 95% identical to the nucleic acid sequence of SEQ ID NO: 4, optionally wherein the Foxp3 promoter has a nucleic acid sequence that is at least 96% identical, 97% identical, 98% identical, 99% identical, or more, to the nucleic acid sequence of SEQ ID NO: 4.

237. The pharmaceutical composition of embodiment 236, wherein the Foxp3 promoter has the nucleic acid sequence of SEQ ID NO: 4.

238. The pharmaceutical composition of any one of embodiments 221-237, wherein the Foxp3 promoter specifically binds transcription factor Nr4a and/or Foxo.

239. The pharmaceutical composition of any one of embodiments 184-238, wherein the one or more lineage-specific transcription regulatory elements include a CNS1 enhancer.

240. The pharmaceutical composition of embodiment 239, wherein the CNS1 enhancer has a nucleic acid sequence that is at least 85% identical to the nucleic acid sequence of SEQ ID NO: 5.

241. The pharmaceutical composition of embodiment 240, wherein the CNS1 enhancer has a nucleic acid sequence that is at least 90% identical to the nucleic acid sequence of SEQ ID NO: 5.

242. The pharmaceutical composition of embodiment 241, wherein the CNS1 enhancer has a nucleic acid sequence that is at least 95% identical to the nucleic acid sequence of SEQ ID NO: 5, optionally wherein the CNS1 enhancer has a nucleic acid sequence that is at least 96% identical, 97% identical, 98% identical, 99% identical, or more, to the nucleic acid sequence of SEQ ID NO: 5.

243. The pharmaceutical composition of embodiment 242, wherein the CNS1 enhancer has the nucleic acid sequence of SEQ ID NO: 5.

244. The pharmaceutical composition of embodiment 239, wherein the CNS1 enhancer has a nucleic acid sequence that is at least 85% identical to the nucleic acid sequence of SEQ ID NO: 6.

245. The pharmaceutical composition of embodiment 244, wherein the CNS1 enhancer has a nucleic acid sequence that is at least 90% identical to the nucleic acid sequence of SEQ ID NO: 6.

246. The pharmaceutical composition of embodiment 245, wherein the CNS1 enhancer has a nucleic acid sequence that is at least 95% identical to the nucleic acid sequence of SEQ ID NO: 6, optionally wherein the CNS1 enhancer has a nucleic acid sequence that is at least 96% identical, 97% identical, 98% identical, 99% identical, or more, to the nucleic acid sequence of SEQ ID NO: 6.

247. The pharmaceutical composition of embodiment 246, wherein the CNS1 enhancer has the nucleic acid sequence of SEQ ID NO: 6.

248. The pharmaceutical composition of embodiment 239, wherein the CNS1 enhancer has a nucleic acid sequence that is at least 85% identical to the nucleic acid sequence of SEQ ID NO: 7.

249. The pharmaceutical composition of embodiment 248, wherein the CNS1 enhancer has a nucleic acid sequence that is at least 90% identical to the nucleic acid sequence of SEQ ID NO: 7.

250. The pharmaceutical composition of embodiment 249, wherein the CNS1 enhancer has a nucleic acid sequence that is at least 95% identical to the nucleic acid sequence of SEQ ID NO: 7, optionally wherein the CNS1 enhancer has a nucleic acid sequence that is at least 96% identical, 97% identical, 98% identical, 99% identical, or more, to the nucleic acid sequence of SEQ ID NO: 7.

251. The pharmaceutical composition of embodiment 250, wherein the CNS1 enhancer has the nucleic acid sequence of SEQ ID NO: 7.

252. The pharmaceutical composition of embodiment 239, wherein the CNS1 enhancer has a nucleic acid sequence that is at least 85% identical to the nucleic acid sequence of SEQ ID NO: 8.

253. The pharmaceutical composition of embodiment 252, wherein the CNS1 enhancer has a nucleic acid sequence that is at least 90% identical to the nucleic acid sequence of SEQ ID NO: 8.

254. The pharmaceutical composition of embodiment 253, wherein the CNS1 enhancer has a nucleic acid sequence that is at least 95% identical to the nucleic acid sequence of SEQ ID NO: 8, optionally wherein the CNS1 enhancer has a nucleic acid sequence that is at least 96% identical, 97% identical, 98% identical, 99% identical, or more, to the nucleic acid sequence of SEQ ID NO: 8.

255. The pharmaceutical composition of embodiment 254, wherein the CNS1 enhancer has the nucleic acid sequence of SEQ ID NO: 8.

256. The pharmaceutical composition of any one of embodiments 239-255, wherein the CNS1 enhancer specifically binds transcription factor AP-1, NFAT, Smad3, and/or Foxo.

257. The pharmaceutical composition of any one of embodiments 184-256, wherein the one or more lineage-specific transcription regulatory elements include a CNS2 enhancer.

258. The pharmaceutical composition of embodiment 257, wherein the CNS2 enhancer has a nucleic acid sequence that is at least 85% identical to the nucleic acid sequence of SEQ ID NO: 9.

259. The pharmaceutical composition of embodiment 258, wherein the CNS2 enhancer has a nucleic acid sequence that is at least 90% identical to the nucleic acid sequence of SEQ ID NO: 9.

260. The pharmaceutical composition of embodiment 259, wherein the CNS2 enhancer has a nucleic acid sequence that is at least 95% identical to the nucleic acid sequence of SEQ ID NO: 9, optionally wherein the CNS2 enhancer has a nucleic acid sequence that is at least 96% identical, 97% identical, 98% identical, 99% identical, or more, to the nucleic acid sequence of SEQ ID NO: 9.

261. The pharmaceutical composition of embodiment 260, wherein the CNS2 enhancer has the nucleic acid sequence of SEQ ID NO: 9.

262. The pharmaceutical composition of embodiment 257, wherein the CNS2 enhancer has a nucleic acid sequence that is at least 85% identical to the nucleic acid sequence of SEQ ID NO: 10.

263. The pharmaceutical composition of embodiment 262, wherein the CNS2 enhancer has a nucleic acid sequence that is at least 90% identical to the nucleic acid sequence of SEQ ID NO: 10.

264. The pharmaceutical composition of embodiment 263, wherein the CNS2 enhancer has a nucleic acid sequence that is at least 95% identical to the nucleic acid sequence of SEQ ID NO: 10, optionally wherein the CNS2 enhancer has a nucleic acid sequence that is at least 96% identical, 97% identical, 98% identical, 99% identical, or more, to the nucleic acid sequence of SEQ ID NO: 10.

265. The pharmaceutical composition of embodiment 264, wherein the CNS2 enhancer has the nucleic acid sequence of SEQ ID NO: 10.

266. The pharmaceutical composition of embodiment 257, wherein the CNS2 enhancer has a nucleic acid sequence that is at least 85% identical to the nucleic acid sequence of SEQ ID NO: 11.

267. The pharmaceutical composition of embodiment 266, wherein the CNS2 enhancer has a nucleic acid sequence that is at least 90% identical to the nucleic acid sequence of SEQ ID NO: 11.

268. The pharmaceutical composition of embodiment 267, wherein the CNS2 enhancer has a nucleic acid sequence that is at least 95% identical to the nucleic acid sequence of SEQ ID NO: 11, optionally wherein the CNS2 enhancer has a nucleic acid sequence that is at least 96% identical, 97% identical, 98% identical, 99% identical, or more, to the nucleic acid sequence of SEQ ID NO: 11.

269. The pharmaceutical composition of embodiment 268, wherein the CNS2 enhancer has the nucleic acid sequence of SEQ ID NO: 11.

270. The pharmaceutical composition of embodiment 257, wherein the CNS2 enhancer has a nucleic acid sequence that is at least 85% identical to the nucleic acid sequence of SEQ ID NO: 12.

271. The pharmaceutical composition of embodiment 270, wherein the CNS2 enhancer has a nucleic acid sequence that is at least 90% identical to the nucleic acid sequence of SEQ ID NO: 12.

272. The pharmaceutical composition of embodiment 271, wherein the CNS2 enhancer has a nucleic acid sequence that is at least 95% identical to the nucleic acid sequence of SEQ ID NO: 12, optionally wherein the CNS2 enhancer has a nucleic acid sequence that is at least 96% identical, 97% identical, 98% identical, 99% identical, or more, to the nucleic acid sequence of SEQ ID NO: 12.

273. The pharmaceutical composition of embodiment 272, wherein the CNS2 enhancer has the nucleic acid sequence of SEQ ID NO: 12.

274. The pharmaceutical composition of any one of embodiments 257-273, wherein the CNS2 enhancer specifically binds transcription factor Runx, Foxp3, Ets-1, CREB, Stat5, NFAT, and/or c-Rel.

275. The pharmaceutical composition of any one of embodiments 184-274, wherein the one or more lineage-specific transcription regulatory elements include a CNS3 enhancer.

276. The pharmaceutical composition of embodiment 275, wherein the CNS3 enhancer has a nucleic acid sequence that is at least 85% identical to the nucleic acid sequence of SEQ ID NO: 13.

277. The pharmaceutical composition of embodiment 276, wherein the CNS3 enhancer has a nucleic acid sequence that is at least 90% identical to the nucleic acid sequence of SEQ ID NO: 13.

278. The pharmaceutical composition of embodiment 277, wherein the CNS3 enhancer has a nucleic acid sequence that is at least 95% identical to the nucleic acid sequence of SEQ ID NO: 13, optionally wherein the CNS3 enhancer has a nucleic acid sequence that is at least 96% identical, 97% identical, 98% identical, 99% identical, or more, to the nucleic acid sequence of SEQ ID NO: 13.

279. The pharmaceutical composition of embodiment 278, wherein the CNS3 enhancer has the nucleic acid sequence of SEQ ID NO: 13.

280. The pharmaceutical composition of embodiment 275, wherein the CNS3 enhancer has a nucleic acid sequence that is at least 85% identical to the nucleic acid sequence of SEQ ID NO: 14.

281. The pharmaceutical composition of embodiment 280, wherein the CNS3 enhancer has a nucleic acid sequence that is at least 90% identical to the nucleic acid sequence of SEQ ID NO: 14.

282. The pharmaceutical composition of embodiment 281, wherein the CNS3 enhancer has a nucleic acid sequence that is at least 95% identical to the nucleic acid sequence of SEQ ID NO: 14, optionally wherein the CNS3 enhancer has a nucleic acid sequence that is at least 96% identical, 97% identical, 98% identical, 99% identical, or more, to the nucleic acid sequence of SEQ ID NO: 14.

283. The pharmaceutical composition of embodiment 282, wherein the CNS3 enhancer has the nucleic acid sequence of SEQ ID NO: 14.

284. The pharmaceutical composition of embodiment 275, wherein the CNS3 enhancer has a nucleic acid sequence that is at least 85% identical to the nucleic acid sequence of SEQ ID NO: 15.

285. The pharmaceutical composition of embodiment 284, wherein the CNS3 enhancer has a nucleic acid sequence that is at least 90% identical to the nucleic acid sequence of SEQ ID NO: 15.

286. The pharmaceutical composition of embodiment 285, wherein the CNS3 enhancer has a nucleic acid sequence that is at least 95% identical to the nucleic acid sequence of SEQ ID NO: 15, optionally wherein the CNS3 enhancer has a nucleic acid sequence that is at least 96% identical, 97% identical, 98% identical, 99% identical, or more, to the nucleic acid sequence of SEQ ID NO: 15.

287. The pharmaceutical composition of embodiment 286, wherein the CNS3 enhancer has the nucleic acid sequence of SEQ ID NO: 15.

288. The pharmaceutical composition of embodiment 275, wherein the CNS3 enhancer has a nucleic acid sequence that is at least 85% identical to the nucleic acid sequence of SEQ ID NO: 16.

289. The pharmaceutical composition of embodiment 288, wherein the CNS3 enhancer has a nucleic acid sequence that is at least 90% identical to the nucleic acid sequence of SEQ ID NO: 16.

290. The pharmaceutical composition of embodiment 289, wherein the CNS3 enhancer has a nucleic acid sequence that is at least 95% identical to the nucleic acid sequence of SEQ ID NO: 16, optionally wherein the CNS3 enhancer has a nucleic acid sequence that is at least 96% identical, 97% identical, 98% identical, 99% identical, or more, to the nucleic acid sequence of SEQ ID NO: 16.

291. The pharmaceutical composition of embodiment 290, wherein the CNS3 enhancer has the nucleic acid sequence of SEQ ID NO: 16.

292. The pharmaceutical composition of any one of embodiments 275-291, wherein the CNS3 enhancer specifically binds transcription factor Foxo and/or c-Rel.

293. The pharmaceutical composition of any one of embodiments 184-292, wherein the one or more lineage-specific transcription regulatory elements include a CNS0 enhancer.

294. The pharmaceutical composition of embodiment 293, wherein the CNS0 enhancer has a nucleic acid sequence that is at least 85% identical to the nucleic acid sequence of SEQ ID NO: 17.

295. The pharmaceutical composition of embodiment 294, wherein the CNS0 enhancer has a nucleic acid sequence that is at least 90% identical to the nucleic acid sequence of SEQ ID NO: 17.

296. The pharmaceutical composition of embodiment 295, wherein the CNS0 enhancer has a nucleic acid sequence that is at least 95% identical to the nucleic acid sequence of SEQ ID NO: 17, optionally wherein the CNS0 enhancer has a nucleic acid sequence that is at least 96% identical, 97% identical, 98% identical, 99% identical, or more, to the nucleic acid sequence of SEQ ID NO: 17.

297. The pharmaceutical composition of embodiment 296, wherein the CNS0 enhancer has the nucleic acid sequence of SEQ ID NO: 17.

298. The pharmaceutical composition of embodiment 293, wherein the CNS0 enhancer has a nucleic acid sequence that is at least 85% identical to the nucleic acid sequence of SEQ ID NO: 18.

299. The pharmaceutical composition of embodiment 298, wherein the CNS0 enhancer has a nucleic acid sequence that is at least 90% identical to the nucleic acid sequence of SEQ ID NO: 18.

300. The pharmaceutical composition of embodiment 299, wherein the CNS0 enhancer has a nucleic acid sequence that is at least 95% identical to the nucleic acid sequence of SEQ ID NO: 18, optionally wherein the CNS0 enhancer has a nucleic acid sequence that is at least 96% identical, 97% identical, 98% identical, 99% identical, or more, to the nucleic acid sequence of SEQ ID NO: 18.

301. The pharmaceutical composition of embodiment 300, wherein the CNS0 enhancer has the nucleic acid sequence of SEQ ID NO: 18.

302. The pharmaceutical composition of embodiment 293, wherein the CNS0 enhancer has a nucleic acid sequence that is at least 85% identical to the nucleic acid sequence of SEQ ID NO: 19.

303. The pharmaceutical composition of embodiment 302, wherein the CNS0 enhancer has a nucleic acid sequence that is at least 90% identical to the nucleic acid sequence of SEQ ID NO: 19.

304. The pharmaceutical composition of embodiment 303, wherein the CNS0 enhancer has a nucleic acid sequence that is at least 95% identical to the nucleic acid sequence of SEQ ID NO: 19, optionally wherein the CNS0 enhancer has a nucleic acid sequence that is at least 96% identical, 97% identical, 98% identical, 99% identical, or more, to the nucleic acid sequence of SEQ ID NO: 19.

305. The pharmaceutical composition of embodiment 304, wherein the CNS0 enhancer has the nucleic acid sequence of SEQ ID NO: 19.

306. The pharmaceutical composition of embodiment 293, wherein the CNS0 enhancer has a nucleic acid sequence that is at least 85% identical to the nucleic acid sequence of SEQ ID NO: 20.

307. The pharmaceutical composition of embodiment 306, wherein the CNS0 enhancer has a nucleic acid sequence that is at least 90% identical to the nucleic acid sequence of SEQ ID NO: 20.

308. The pharmaceutical composition of embodiment 307, wherein the CNS0 enhancer has a nucleic acid sequence that is at least 95% identical to the nucleic acid sequence of SEQ ID NO: 20, optionally wherein the CNS0 enhancer has a nucleic acid sequence that is at least 96% identical, 97% identical, 98% identical, 99% identical, or more, to the nucleic acid sequence of SEQ ID NO: 20.

309. The pharmaceutical composition of embodiment 308, wherein the CNS0 enhancer has the nucleic acid sequence of SEQ ID NO: 20.

310. The pharmaceutical composition of any one of embodiments 293-309, wherein the CNS0 enhancer specifically binds transcription factor Satb1 and/or Stat5.

311. The pharmaceutical composition of any one of embodiments 184-310, wherein the nucleic acid cassette is operably linked to a riboswitch.

312. The pharmaceutical composition of embodiment 311, wherein binding of a ligand to the riboswitch induces expression of the nucleic acid cassette.

313. The pharmaceutical composition of any one of embodiments 184-312, wherein the autoantigen-binding protein is a single-chain polypeptide.

314. The pharmaceutical composition of any one of embodiments 184-313, wherein the autoantigen-binding protein is a CAR.

315. The pharmaceutical composition of embodiment 314, wherein the chimeric antigen receptor includes an antigen recognition domain, a hinge domain, a transmembrane domain, and one or more intracellular signaling domains.

316. The pharmaceutical composition of embodiment 315, wherein the one or more intracellular signaling domains include one or more primary intracellular signaling domains and optionally one or more costimulatory intracellular signaling domains.

317. The pharmaceutical composition of embodiment 315 or 316, wherein the antigen recognition domain is a single-chain antibody fragment, optionally wherein the single-chain antibody fragment is an scFv.

318. The pharmaceutical composition of any one of embodiments 315-317, wherein the hinge domain is a CD28, CD8, IgG1/IgG4, CD4, CD7, or IgD hinge domain.

319. The pharmaceutical composition of embodiment 318, wherein the hinge domain is a CD28 hinge domain.

320. The pharmaceutical composition of any one of embodiments 315-319, wherein the transmembrane domain includes a CD28, CD3 zeta, CD8, FcRIγ, CD4, CD7, OX40, or MHC (H2-Kb) transmembrane domain.

321. The pharmaceutical composition of embodiment 320, wherein the transmembrane domain includes a CD28 transmembrane domain.

322. The pharmaceutical composition of any one of embodiments 316-321, wherein the one or more primary intracellular signaling domains are selected from the group consisting of a CD3 zeta, FcR gamma, FcR beta, CD3 gamma, CD3 delta, CD3 epsilon, CD5, CD22, CD79a, CD79b, CD278 (ICOS), CD66d, DAP10, and a DAP12 intracellular signaling domain.

323. The pharmaceutical composition of embodiment 322, wherein at least one of the one or more primary intracellular signaling domains is a CD3 zeta intracellular signaling domain.

324. The pharmaceutical composition of any one of embodiments 316-323, wherein the one or more costimulatory intracellular signaling domains are selected from the group consisting of a CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H₃, CD83, CDS, ICAM-1, LFA-1 (CD11 a/CD18), an MHC class I molecule, BTLA, and a Toll ligand receptor intracellular signaling domain.

325. The pharmaceutical composition of embodiment 324, wherein at least one of the one or more co-stimulatory intracellular signaling domains is a CD28 intracellular signaling domain.

326. The pharmaceutical composition of any one of embodiments 315-325, wherein the chimeric antigen receptor includes an N-terminal leader sequence.

327. The pharmaceutical composition of any one of embodiments 315-326, wherein the antigen recognition domain includes an N-terminal leader sequence.

328. The pharmaceutical composition of embodiment 327, wherein the N-terminal leader sequence of the antigen recognition domain is cleaved from the antigen recognition domain during cellular processing and localization of the chimeric antigen receptor to the cellular membrane.

329. The pharmaceutical composition of any one of embodiments 184-312, wherein the autoantigen-binding protein is a multi-chain protein.

330. The pharmaceutical composition of embodiment 329, wherein the autoantigen-binding protein is a full-length antibody, a dual-variable immunoglobulin domain, a diabody, a triabody, an antibody-like protein scaffold, a Fab fragment, or a F(ab′)₂ molecule.

331. The pharmaceutical composition of any one of embodiments 184-330, wherein the autoantigen is myelin oligodendrocyte glycoprotein, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double stranded DNA, single stranded DNA, transferrin, fetuin, factor II, factor VII, fibrin, fibrinogen, C1, C1q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

332. A kit that includes the pharmaceutical composition of any one of embodiments 184-331, wherein the kit further includes a package insert instructing a user of the kit to administer the pharmaceutical composition to a human patient having an autoimmune disease.

333. The kit of embodiment 332, wherein the package insert instructs a user of the kit to perform the method of any one of embodiments 1-183.

334. A nucleic acid cassette encoding an autoantigen-binding protein, wherein the nucleic acid cassette is operably linked to one or more lineage-specific transcription regulatory elements that are active in CD4+CD25+ Treg cells (i.e., specifically active in cells of the Treg lineage and not active in other cell types (e.g., other hematopoietic cells)).

335. The nucleic acid cassette of embodiment 334, wherein the one or more lineage-specific transcription regulatory elements include a Foxp3 promoter.

336. The nucleic acid cassette of embodiment 335, wherein the Foxp3 promoter has a nucleic acid sequence that is at least 85% identical to the nucleic acid sequence of SEQ ID NO: 1.

337. The nucleic acid cassette of embodiment 336, wherein the Foxp3 promoter has a nucleic acid sequence that is at least 90% identical to the nucleic acid sequence of SEQ ID NO: 1.

338. The nucleic acid cassette of embodiment 337, wherein the Foxp3 promoter has a nucleic acid sequence that is at least 95% identical to the nucleic acid sequence of SEQ ID NO: 1, optionally wherein the Foxp3 promoter has a nucleic acid sequence that is at least 96% identical, 97% identical, 98% identical, 99% identical, or more, to the nucleic acid sequence of SEQ ID NO: 1.

339. The nucleic acid cassette of embodiment 338, wherein the Foxp3 promoter has the nucleic acid sequence of SEQ ID NO: 1.

340. The nucleic acid cassette of embodiment 335, wherein the Foxp3 promoter has a nucleic acid sequence that is at least 85% identical to the nucleic acid sequence of SEQ ID NO: 2.

341. The nucleic acid cassette of embodiment 340, wherein the Foxp3 promoter has a nucleic acid sequence that is at least 90% identical to the nucleic acid sequence of SEQ ID NO: 2.

342. The nucleic acid cassette of embodiment 341, wherein the Foxp3 promoter has a nucleic acid sequence that is at least 95% identical to the nucleic acid sequence of SEQ ID NO: 2, optionally wherein the Foxp3 promoter has a nucleic acid sequence that is at least 96% identical, 97% identical, 98% identical, 99% identical, or more, to the nucleic acid sequence of SEQ ID NO: 2.

343. The nucleic acid cassette of embodiment 342, wherein the Foxp3 promoter has the nucleic acid sequence of SEQ ID NO: 2.

344. The nucleic acid cassette of embodiment 335, wherein the Foxp3 promoter has a nucleic acid sequence that is at least 85% identical to the nucleic acid sequence of SEQ ID NO: 3.

345. The nucleic acid cassette of embodiment 344, wherein the Foxp3 promoter has a nucleic acid sequence that is at least 90% identical to the nucleic acid sequence of SEQ ID NO: 3.

346. The nucleic acid cassette of embodiment 345, wherein the Foxp3 promoter has a nucleic acid sequence that is at least 95% identical to the nucleic acid sequence of SEQ ID NO: 3, optionally wherein the Foxp3 promoter has a nucleic acid sequence that is at least 96% identical, 97% identical, 98% identical, 99% identical, or more, to the nucleic acid sequence of SEQ ID NO: 3.

347. The nucleic acid cassette of embodiment 346, wherein the Foxp3 promoter has the nucleic acid sequence of SEQ ID NO: 3.

348. The nucleic acid cassette of embodiment 335, wherein the Foxp3 promoter has a nucleic acid sequence that is at least 85% identical to the nucleic acid sequence of SEQ ID NO: 4.

349. The nucleic acid cassette of embodiment 348, wherein the Foxp3 promoter has a nucleic acid sequence that is at least 90% identical to the nucleic acid sequence of SEQ ID NO: 4.

350. The nucleic acid cassette of embodiment 349, wherein the Foxp3 promoter has a nucleic acid sequence that is at least 95% identical to the nucleic acid sequence of SEQ ID NO: 4, optionally wherein the Foxp3 promoter has a nucleic acid sequence that is at least 96% identical, 97% identical, 98% identical, 99% identical, or more, to the nucleic acid sequence of SEQ ID NO: 4.

351. The nucleic acid cassette of embodiment 350, wherein the Foxp3 promoter has the nucleic acid sequence of SEQ ID NO: 4.

352. The nucleic acid cassette of any one of embodiments 335-351, wherein the Foxp3 promoter specifically binds transcription factor Nr4a and/or Foxo.

353. The nucleic acid cassette of any one of embodiments 334-352, wherein the one or more lineage-specific transcription regulatory elements include a CNS1 enhancer.

354. The nucleic acid cassette of embodiment 353, wherein the CNS1 enhancer has a nucleic acid sequence that is at least 85% identical to the nucleic acid sequence of SEQ ID NO: 5.

355. The nucleic acid cassette of embodiment 354, wherein the CNS1 enhancer has a nucleic acid sequence that is at least 90% identical to the nucleic acid sequence of SEQ ID NO: 5.

356. The nucleic acid cassette of embodiment 355, wherein the CNS1 enhancer has a nucleic acid sequence that is at least 95% identical to the nucleic acid sequence of SEQ ID NO: 5, optionally wherein the CNS1 enhancer has a nucleic acid sequence that is at least 96% identical, 97% identical, 98% identical, 99% identical, or more, to the nucleic acid sequence of SEQ ID NO: 5.

357. The nucleic acid cassette of embodiment 356, wherein the CNS1 enhancer has the nucleic acid sequence of SEQ ID NO: 5.

358. The nucleic acid cassette of embodiment 353, wherein the CNS1 enhancer has a nucleic acid sequence that is at least 85% identical to the nucleic acid sequence of SEQ ID NO: 6.

359. The nucleic acid cassette of embodiment 358, wherein the CNS1 enhancer has a nucleic acid sequence that is at least 90% identical to the nucleic acid sequence of SEQ ID NO: 6.

360. The nucleic acid cassette of embodiment 359, wherein the CNS1 enhancer has a nucleic acid sequence that is at least 95% identical to the nucleic acid sequence of SEQ ID NO: 6, optionally wherein the CNS1 enhancer has a nucleic acid sequence that is at least 96% identical, 97% identical, 98% identical, 99% identical, or more, to the nucleic acid sequence of SEQ ID NO: 6.

361. The nucleic acid cassette of embodiment 360, wherein the CNS1 enhancer has the nucleic acid sequence of SEQ ID NO: 6.

362. The nucleic acid cassette of embodiment 353, wherein the CNS1 enhancer has a nucleic acid sequence that is at least 85% identical to the nucleic acid sequence of SEQ ID NO: 7.

363. The nucleic acid cassette of embodiment 362, wherein the CNS1 enhancer has a nucleic acid sequence that is at least 90% identical to the nucleic acid sequence of SEQ ID NO: 7.

364. The nucleic acid cassette of embodiment 363, wherein the CNS1 enhancer has a nucleic acid sequence that is at least 95% identical to the nucleic acid sequence of SEQ ID NO: 7, optionally wherein the CNS1 enhancer has a nucleic acid sequence that is at least 96% identical, 97% identical, 98% identical, 99% identical, or more, to the nucleic acid sequence of SEQ ID NO: 7.

365. The nucleic acid cassette of embodiment 364, wherein the CNS1 enhancer has the nucleic acid sequence of SEQ ID NO: 7.

366. The nucleic acid cassette of embodiment 353, wherein the CNS1 enhancer has a nucleic acid sequence that is at least 85% identical to the nucleic acid sequence of SEQ ID NO: 8.

367. The nucleic acid cassette of embodiment 366, wherein the CNS1 enhancer has a nucleic acid sequence that is at least 90% identical to the nucleic acid sequence of SEQ ID NO: 8.

368. The nucleic acid cassette of embodiment 367, wherein the CNS1 enhancer has a nucleic acid sequence that is at least 95% identical to the nucleic acid sequence of SEQ ID NO: 8, optionally wherein the CNS1 enhancer has a nucleic acid sequence that is at least 96% identical, 97% identical, 98% identical, 99% identical, or more, to the nucleic acid sequence of SEQ ID NO: 8.

369. The nucleic acid cassette of embodiment 368, wherein the CNS1 enhancer has the nucleic acid sequence of SEQ ID NO: 8.

370. The nucleic acid cassette of any one of embodiments 353-369, wherein the CNS1 enhancer specifically binds transcription factor AP-1, NFAT, Smad3, and/or Foxo.

371. The nucleic acid cassette of any one of embodiments 334-370, wherein the one or more lineage-specific transcription regulatory elements include a CNS2 enhancer.

372. The nucleic acid cassette of embodiment 371, wherein the CNS2 enhancer has a nucleic acid sequence that is at least 85% identical to the nucleic acid sequence of SEQ ID NO: 9.

373. The nucleic acid cassette of embodiment 372, wherein the CNS2 enhancer has a nucleic acid sequence that is at least 90% identical to the nucleic acid sequence of SEQ ID NO: 9.

374. The nucleic acid cassette of embodiment 373, wherein the CNS2 enhancer has a nucleic acid sequence that is at least 95% identical to the nucleic acid sequence of SEQ ID NO: 9, optionally wherein the CNS2 enhancer has a nucleic acid sequence that is at least 96% identical, 97% identical, 98% identical, 99% identical, or more, to the nucleic acid sequence of SEQ ID NO: 9.

375. The nucleic acid cassette of embodiment 374, wherein the CNS2 enhancer has the nucleic acid sequence of SEQ ID NO: 9.

376. The nucleic acid cassette of embodiment 371, wherein the CNS2 enhancer has a nucleic acid sequence that is at least 85% identical to the nucleic acid sequence of SEQ ID NO: 10.

377. The nucleic acid cassette of embodiment 376, wherein the CNS2 enhancer has a nucleic acid sequence that is at least 90% identical to the nucleic acid sequence of SEQ ID NO: 10.

378. The nucleic acid cassette of embodiment 377, wherein the CNS2 enhancer has a nucleic acid sequence that is at least 95% identical to the nucleic acid sequence of SEQ ID NO: 10, optionally wherein the CNS2 enhancer has a nucleic acid sequence that is at least 96% identical, 97% identical, 98% identical, 99% identical, or more, to the nucleic acid sequence of SEQ ID NO: 10.

379. The nucleic acid cassette of embodiment 378, wherein the CNS2 enhancer has the nucleic acid sequence of SEQ ID NO: 10.

380. The nucleic acid cassette of embodiment 371, wherein the CNS2 enhancer has a nucleic acid sequence that is at least 85% identical to the nucleic acid sequence of SEQ ID NO: 11.

381. The nucleic acid cassette of embodiment 380, wherein the CNS2 enhancer has a nucleic acid sequence that is at least 90% identical to the nucleic acid sequence of SEQ ID NO: 11.

382. The nucleic acid cassette of embodiment 381, wherein the CNS2 enhancer has a nucleic acid sequence that is at least 95% identical to the nucleic acid sequence of SEQ ID NO: 11, optionally wherein the CNS2 enhancer has a nucleic acid sequence that is at least 96% identical, 97% identical, 98% identical, 99% identical, or more, to the nucleic acid sequence of SEQ ID NO: 11.

383. The nucleic acid cassette of embodiment 382, wherein the CNS2 enhancer has the nucleic acid sequence of SEQ ID NO: 11.

384. The nucleic acid cassette of embodiment 371, wherein the CNS2 enhancer has a nucleic acid sequence that is at least 85% identical to the nucleic acid sequence of SEQ ID NO: 12.

385. The nucleic acid cassette of embodiment 384, wherein the CNS2 enhancer has a nucleic acid sequence that is at least 90% identical to the nucleic acid sequence of SEQ ID NO: 12.

386. The nucleic acid cassette of embodiment 385, wherein the CNS2 enhancer has a nucleic acid sequence that is at least 95% identical to the nucleic acid sequence of SEQ ID NO: 12, optionally wherein the CNS2 enhancer has a nucleic acid sequence that is at least 96% identical, 97% identical, 98% identical, 99% identical, or more, to the nucleic acid sequence of SEQ ID NO: 12.

387. The nucleic acid cassette of embodiment 386, wherein the CNS2 enhancer has the nucleic acid sequence of SEQ ID NO: 12.

388. The nucleic acid cassette of any one of embodiments 371-387, wherein the CNS2 enhancer specifically binds transcription factor Runx, Foxp3, Ets-1, CREB, Stat5, NFAT, and/or c-Rel.

389. The nucleic acid cassette of any one of embodiments 334-388, wherein the one or more lineage-specific transcription regulatory elements include a CNS3 enhancer.

390. The nucleic acid cassette of embodiment 389, wherein the CNS3 enhancer has a nucleic acid sequence that is at least 85% identical to the nucleic acid sequence of SEQ ID NO: 13.

391. The nucleic acid cassette of embodiment 390, wherein the CNS3 enhancer has a nucleic acid sequence that is at least 90% identical to the nucleic acid sequence of SEQ ID NO: 13.

392. The nucleic acid cassette of embodiment 391, wherein the CNS3 enhancer has a nucleic acid sequence that is at least 95% identical to the nucleic acid sequence of SEQ ID NO: 13, optionally wherein the CNS3 enhancer has a nucleic acid sequence that is at least 96% identical, 97% identical, 98% identical, 99% identical, or more, to the nucleic acid sequence of SEQ ID NO: 13.

393. The nucleic acid cassette of embodiment 392, wherein the CNS3 enhancer has the nucleic acid sequence of SEQ ID NO: 13.

394. The nucleic acid cassette of embodiment 389, wherein the CNS3 enhancer has a nucleic acid sequence that is at least 85% identical to the nucleic acid sequence of SEQ ID NO: 14.

395. The nucleic acid cassette of embodiment 394, wherein the CNS3 enhancer has a nucleic acid sequence that is at least 90% identical to the nucleic acid sequence of SEQ ID NO: 14.

396. The nucleic acid cassette of embodiment 395, wherein the CNS3 enhancer has a nucleic acid sequence that is at least 95% identical to the nucleic acid sequence of SEQ ID NO: 14, optionally wherein the CNS3 enhancer has a nucleic acid sequence that is at least 96% identical, 97% identical, 98% identical, 99% identical, or more, to the nucleic acid sequence of SEQ ID NO: 14.

397. The nucleic acid cassette of embodiment 396, wherein the CNS3 enhancer has the nucleic acid sequence of SEQ ID NO: 14.

398. The nucleic acid cassette of embodiment 389, wherein the CNS3 enhancer has a nucleic acid sequence that is at least 85% identical to the nucleic acid sequence of SEQ ID NO: 15.

399. The nucleic acid cassette of embodiment 398, wherein the CNS3 enhancer has a nucleic acid sequence that is at least 90% identical to the nucleic acid sequence of SEQ ID NO: 15.

400. The nucleic acid cassette of embodiment 399, wherein the CNS3 enhancer has a nucleic acid sequence that is at least 95% identical to the nucleic acid sequence of SEQ ID NO: 15, optionally wherein the CNS3 enhancer has a nucleic acid sequence that is at least 96% identical, 97% identical, 98% identical, 99% identical, or more, to the nucleic acid sequence of SEQ ID NO: 15.

401. The nucleic acid cassette of embodiment 400, wherein the CNS3 enhancer has the nucleic acid sequence of SEQ ID NO: 15.

402. The nucleic acid cassette of embodiment 389, wherein the CNS3 enhancer has a nucleic acid sequence that is at least 85% identical to the nucleic acid sequence of SEQ ID NO: 16.

403. The nucleic acid cassette of embodiment 402, wherein the CNS3 enhancer has a nucleic acid sequence that is at least 90% identical to the nucleic acid sequence of SEQ ID NO: 16.

404. The nucleic acid cassette of embodiment 403, wherein the CNS3 enhancer has a nucleic acid sequence that is at least 95% identical to the nucleic acid sequence of SEQ ID NO: 16, optionally wherein the CNS3 enhancer has a nucleic acid sequence that is at least 96% identical, 97% identical, 98% identical, 99% identical, or more, to the nucleic acid sequence of SEQ ID NO: 16.

405. The nucleic acid cassette of embodiment 404, wherein the CNS3 enhancer has the nucleic acid sequence of SEQ ID NO: 16.

406. The nucleic acid cassette of any one of embodiments 389-405, wherein the CNS3 enhancer specifically binds transcription factor Foxo and/or c-Rel.

407. The nucleic acid cassette of any one of embodiments 334-406, wherein the one or more lineage-specific transcription regulatory elements include a CNS0 enhancer.

408. The nucleic acid cassette of embodiment 407, wherein the CNS0 enhancer has a nucleic acid sequence that is at least 85% identical to the nucleic acid sequence of SEQ ID NO: 17.

409. The nucleic acid cassette of embodiment 408, wherein the CNS0 enhancer has a nucleic acid sequence that is at least 90% identical to the nucleic acid sequence of SEQ ID NO: 17.

410. The nucleic acid cassette of embodiment 409, wherein the CNS0 enhancer has a nucleic acid sequence that is at least 95% identical to the nucleic acid sequence of SEQ ID NO: 17, optionally wherein the CNS0 enhancer has a nucleic acid sequence that is at least 96% identical, 97% identical, 98% identical, 99% identical, or more, to the nucleic acid sequence of SEQ ID NO: 17.

411. The nucleic acid cassette of embodiment 410, wherein the CNS0 enhancer has the nucleic acid sequence of SEQ ID NO: 17.

412. The nucleic acid cassette of embodiment 407, wherein the CNS0 enhancer has a nucleic acid sequence that is at least 85% identical to the nucleic acid sequence of SEQ ID NO: 18.

413. The nucleic acid cassette of embodiment 412, wherein the CNS0 enhancer has a nucleic acid sequence that is at least 90% identical to the nucleic acid sequence of SEQ ID NO: 18.

414. The nucleic acid cassette of embodiment 413, wherein the CNS0 enhancer has a nucleic acid sequence that is at least 95% identical to the nucleic acid sequence of SEQ ID NO: 18, optionally wherein the CNS0 enhancer has a nucleic acid sequence that is at least 96% identical, 97% identical, 98% identical, 99% identical, or more, to the nucleic acid sequence of SEQ ID NO: 18.

415. The nucleic acid cassette of embodiment 414, wherein the CNS0 enhancer has the nucleic acid sequence of SEQ ID NO: 18.

416. The nucleic acid cassette of embodiment 407, wherein the CNS0 enhancer has a nucleic acid sequence that is at least 85% identical to the nucleic acid sequence of SEQ ID NO: 19.

417. The nucleic acid cassette of embodiment 416, wherein the CNS0 enhancer has a nucleic acid sequence that is at least 90% identical to the nucleic acid sequence of SEQ ID NO: 19.

418. The nucleic acid cassette of embodiment 417, wherein the CNS0 enhancer has a nucleic acid sequence that is at least 95% identical to the nucleic acid sequence of SEQ ID NO: 19, optionally wherein the CNS0 enhancer has a nucleic acid sequence that is at least 96% identical, 97% identical, 98% identical, 99% identical, or more, to the nucleic acid sequence of SEQ ID NO: 19.

419. The nucleic acid cassette of embodiment 418, wherein the CNS0 enhancer has the nucleic acid sequence of SEQ ID NO: 19.

420. The nucleic acid cassette of embodiment 407, wherein the CNS0 enhancer has a nucleic acid sequence that is at least 85% identical to the nucleic acid sequence of SEQ ID NO: 20.

421. The nucleic acid cassette of embodiment 420, wherein the CNS0 enhancer has a nucleic acid sequence that is at least 90% identical to the nucleic acid sequence of SEQ ID NO: 20.

422. The nucleic acid cassette of embodiment 421, wherein the CNS0 enhancer has a nucleic acid sequence that is at least 95% identical to the nucleic acid sequence of SEQ ID NO: 20, optionally wherein the CNS0 enhancer has a nucleic acid sequence that is at least 96% identical, 97% identical, 98% identical, 99% identical, or more, to the nucleic acid sequence of SEQ ID NO: 20.

423. The nucleic acid cassette of embodiment 422, wherein the CNS0 enhancer has the nucleic acid sequence of SEQ ID NO: 20.

424. The nucleic acid cassette of any one of embodiments 407-423, wherein the CNS0 enhancer specifically binds transcription factor Satb1 and/or Stat5.

425. The nucleic acid cassette of any one of embodiments 334-424, wherein the nucleic acid cassette is operably linked to a riboswitch.

426. The nucleic acid cassette of embodiment 425, wherein binding of a ligand to the riboswitch induces expression of the nucleic acid cassette.

427. The nucleic acid cassette of any one of embodiments 334-426, wherein the autoantigen-binding protein is a single-chain polypeptide.

428. The nucleic acid cassette of any one of embodiments 334-427, wherein the autoantigen-binding protein is a CAR.

429. The nucleic acid cassette of embodiment 428, wherein the chimeric antigen receptor includes an antigen recognition domain, a hinge domain, a transmembrane domain, and one or more intracellular signaling domains.

430. The nucleic acid cassette of embodiment 429, wherein the one or more intracellular signaling domains include one or more primary intracellular signaling domains and optionally one or more costimulatory intracellular signaling domains.

431. The nucleic acid cassette of embodiment 429 or 430, wherein the antigen recognition domain is a single-chain antibody fragment, optionally wherein the single-chain antibody fragment is an scFv.

432. The nucleic acid cassette of any one of embodiments 429-431, wherein the hinge domain is a CD28, CD8, IgG1/IgG4, CD4, CD7, or IgD hinge domain.

433. The nucleic acid cassette of embodiment 432, wherein the hinge domain is a CD28 hinge domain.

434. The nucleic acid cassette of any one of embodiments 429-433, wherein the transmembrane domain includes a CD28, CD3 zeta, CD8, FcRIγ, CD4, CD7, OX40, or MHC (H₂-Kb) transmembrane domain.

435. The nucleic acid cassette of embodiment 434, wherein the transmembrane domain includes a CD28 transmembrane domain.

436. The nucleic acid cassette of any one of embodiments 430-435, wherein the one or more primary intracellular signaling domains are selected from the group consisting of a CD3 zeta, FcR gamma, FcR beta, CD3 gamma, CD3 delta, CD3 epsilon, CD5, CD22, CD79a, CD79b, CD278 (ICOS), CD66d, DAP10, and a DAP12 intracellular signaling domain.

437. The nucleic acid cassette of embodiment 436, wherein at least one of the one or more primary intracellular signaling domains is a CD3 zeta intracellular signaling domain.

438. The nucleic acid cassette of any one of embodiments 430-437, wherein the one or more costimulatory intracellular signaling domains are selected from the group consisting of a CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H₃, CD83, CDS, ICAM-1, LFA-1 (CD11a/CD18), an MHC class I molecule, BTLA, and a Toll ligand receptor intracellular signaling domain.

439. The nucleic acid cassette of embodiment 438, wherein at least one of the one or more co-stimulatory intracellular signaling domains is a CD28 intracellular signaling domain.

440. The nucleic acid cassette of any one of embodiments 429-439, wherein the chimeric antigen receptor includes an N-terminal leader sequence.

441. The nucleic acid cassette of any one of embodiments 429-440, wherein the antigen recognition domain includes an N-terminal leader sequence.

442. The nucleic acid cassette of embodiment 441, wherein the N-terminal leader sequence of the antigen recognition domain is cleaved from the antigen recognition domain during cellular processing and localization of the chimeric antigen receptor to the cellular membrane.

443. The nucleic acid cassette of any one of embodiments 334-426, wherein the autoantigen-binding protein is a multi-chain protein.

444. The nucleic acid cassette of embodiment 443, wherein the autoantigen-binding protein is a full-length antibody, a dual-variable immunoglobulin domain, a diabody, a triabody, an antibody-like protein scaffold, a Fab fragment, or a F(ab′)₂ molecule.

445. The nucleic acid cassette of any one of embodiments 334-444, wherein the autoantigen is myelin oligodendrocyte glycoprotein, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double stranded DNA, single stranded DNA, transferrin, fetuin, factor II, factor VII, fibrin, fibrinogen, C1, C1q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

446. A viral vector that includes the nucleic acid cassette of any one of embodiments 334-445.

447. The viral vector of embodiment 446, wherein the viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

448. The viral vector of embodiment 447, wherein the viral vector is a Retroviridae family viral vector.

449. The viral vector of embodiment 448, wherein the Retroviridae family viral vector is a lentiviral vector.

450. The viral vector of embodiment 448, wherein the Retroviridae family viral vector is an alpharetroviral vector or a gammaretroviral vector.

451. The viral vector of any one of embodiments 447-450, wherein the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3-self inactivating LTR.

452. The viral vector of any one of embodiments 447-451, wherein the viral vector is a pseudotyped viral vector.

453. The viral vector of embodiment 452, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

454. The viral vector of embodiment 453, wherein the pseudotyped viral vector is a pseudotyped lentiviral vector.

455. The viral vector of any one of embodiments 452-454, wherein the pseudotyped viral vector includes an envelope protein from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

456. The viral vector of embodiment 455, wherein the pseudotyped viral vector includes a VSV-G envelope protein.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A method of treating or preventing an autoimmune disease in a patient in need thereof, the method comprising administering to the patient a population of pluripotent hematopoietic cells comprising a nucleic acid cassette that encodes an autoantigen-binding protein, wherein the nucleic acid cassette is operably linked to one or more lineage-specific transcription regulatory elements that are active in CD4+CD25+ regulatory T (Treg) cells.

2. A method of suppressing activity and/or proliferation of a population of autoreactive effector immune cells in a patient diagnosed as having an autoimmune disease, the method comprising administering to the patient a population of pluripotent hematopoietic cells comprising a nucleic acid cassette that encodes an autoantigen-binding protein, wherein the nucleic acid cassette is operably linked to one or more lineage-specific transcription regulatory elements that are active in CD4+CD25+ Treg cells.

3. A method of inducing apoptosis of an autoreactive effector immune cell in a patient diagnosed as having an autoimmune disease, the method comprising administering to the patient a population of pluripotent hematopoietic cells comprising a nucleic acid cassette that encodes an autoantigen-binding protein, wherein the nucleic acid cassette is operably linked to one or more lineage-specific transcription regulatory elements that are active in CD4+CD25+ Treg cells.

4. A method of protecting endogenous tissue from an autoimmune response in a patient diagnosed as having an autoimmune disease, the method comprising administering to the patient a population of pluripotent hematopoietic cells comprising a nucleic acid cassette that encodes an autoantigen-binding protein, wherein the nucleic acid cassette is operably linked to one or more lineage-specific transcription regulatory elements that are active in CD4+CD25+ Treg cells.

5. A method of reducing inflammation in a patient diagnosed as having an autoimmune disease, the method comprising administering to the patient a population of pluripotent hematopoietic cells comprising a nucleic acid cassette that encodes an autoantigen-binding protein, wherein the nucleic acid cassette is operably linked to one or more lineage-specific transcription regulatory elements that are active in CD4+CD25+ Treg cells.

6. The method of any one of claims 1-5, wherein the pluripotent hematopoietic cells are hematopoietic stem cells (HSCs) or hematopoietic progenitor cells (HPCs).

7. The method of any one of claims 1-5, wherein the pluripotent hematopoietic cells are embryonic stem cells.

8. The method of any one of claims 1-5, wherein the pluripotent hematopoietic cells are induced pluripotent stem cells.

9. The method of any one of claims 1-5, wherein the pluripotent hematopoietic cells are lymphoid progenitor cells.

10. The method of any one of claims 1-9, wherein the pluripotent hematopoietic cells are CD34+ cells.

11. The method of any one of claims 1-10, wherein the population of pluripotent hematopoietic cells is administered systemically to the patient.

12. The method of claim 11, wherein the population of pluripotent hematopoietic cells is administered to the patient by way of intravenous injection.

13. The method of any one of claims 1-12, wherein the pluripotent hematopoietic cells are autologous with respect to the patient.

14. The method of any one of claims 1-12, wherein the pluripotent hematopoietic cells are allogeneic with respect to the patient.

15. The method of claim 14, wherein the pluripotent hematopoietic cells are HLA-matched to the patient.

16. The method of any one of claims 1-15, wherein the pluripotent hematopoietic cells are transduced ex vivo with a viral vector comprising the nucleic acid cassette that encodes the autoantigen-binding protein.

17. The method of any one of claims 1-15, wherein the pluripotent hematopoietic cells are transfected ex vivo with a polynucleotide comprising the nucleic acid cassette that encodes the autoantigen-binding protein.

18. The method of any one of claims 1-15, wherein the pluripotent hematopoietic cells are obtained by delivering to the cells a nuclease that catalyzes a single-strand break or a double-strand break at a target position within the genome of the cell.

19. The method of claim 18, wherein the nuclease is delivered to the cells in combination with a guide RNA (gRNA) that hybridizes to the target position within the genome of the cell.

20. The method of claim 18 or 19, wherein the nuclease is a clustered regularly interspaced short palindromic repeats (CRISPR)-associated protein.

21. The method of claim 20, wherein the CRISPR-associated protein is CRISPR-associated protein 9 (Cas9) or CRISPR-associated protein 12a (Cas12a).

22. The method of any one of claims 18-21, wherein while the cells are contacted with the nuclease the cells are additionally contacted with a template polynucleotide comprising the nucleic acid cassette that encodes the autoantigen-binding protein.

23. The method of claim 22, wherein the template polynucleotide comprises a 5′ homology arm and a 3′ homology arm having nucleic acid sequences that are sufficiently similar to the nucleic acid sequences located 5′ to the target position and 3′ to the target position, respectively, to promote homologous recombination.

24. The method of claim 22 or 23, wherein the nuclease, gRNA, and/or template polynucleotide are delivered to the cells by contacting the cells with a viral vector that encodes the nuclease, gRNA, and/or template polynucleotide.

25. The method of any one of claims 1-24, wherein the one or more lineage-specific transcription regulatory elements comprise a Foxp3 promoter.

26. The method of claim 25, wherein the Foxp3 promoter has the nucleic acid sequence of SEQ ID NO: 1, SEQ ID NO: 2, SEQ ID NO: 3, or SEQ ID NO: 4, or a nucleic acid sequence that is at least 85% identical thereto.

27. The method of claim 25 or 26, wherein the Foxp3 promoter specifically binds transcription factor Nr4a and/or Foxo.

28. The method of any one of claims 1-27, wherein the one or more lineage-specific transcription regulatory elements comprise a CNS1 enhancer.

29. The method of claim 28, wherein the CNS1 enhancer has the nucleic acid sequence of SEQ ID NO: 5, SEQ ID NO: 6, SEQ ID NO: 7, or SEQ ID NO: 8, or a nucleic acid sequence that is at least 85% identical thereto.

30. The method of claim 28 or 29, wherein the CNS1 enhancer specifically binds transcription factor AP-1, NFAT, Smad3, and/or Foxo.

31. The method of any one of claims 1-30, wherein the one or more lineage-specific transcription regulatory elements comprise a CNS2 enhancer.

32. The method of claim 31, wherein the CNS2 enhancer has the nucleic acid sequence of SEQ ID NO: 9, SEQ ID NO: 10, SEQ ID NO: 11, or SEQ ID NO: 12, or a nucleic acid sequence that is at least 85% identical thereto.

33. The method of claim 31 or 32, wherein the CNS2 enhancer specifically binds transcription factor Runx, Foxp3, Ets-1, CREB, Stat5, NFAT, and/or c-Rel.

34. The method of any one of claims 1-33, wherein the one or more lineage-specific transcription regulatory elements comprise a CNS3 enhancer.

35. The method of claim 34, wherein the CNS3 enhancer has the nucleic acid sequence of SEQ ID NO: 13, SEQ ID NO: 14, SEQ ID NO: 15, or SEQ ID NO: 16, or a nucleic acid sequence that is at least 85% identical thereto.

36. The method of claim 34 or 35, wherein the CNS3 enhancer specifically binds transcription factor Foxo and/or c-Rel.

37. The method of any one of claims 1-36, wherein the one or more lineage-specific transcription regulatory elements comprise a CNS0 enhancer.

38. The method of claim 37, wherein the CNS0 enhancer has the nucleic acid sequence of SEQ ID NO: 17, SEQ ID NO: 18, SEQ ID NO: 19, or SEQ ID NO: 20, or a nucleic acid sequence that is at least 85% identical thereto.

39. The method of claim 37 or 38, wherein the CNS0 enhancer specifically binds transcription factor Satb1 and/or Stat5.

40. The method of any one of claims 1-39, wherein the autoantigen-binding protein is a single-chain polypeptide.

41. The method of any one of claims 1-40, wherein the autoantigen-binding protein is a chimeric antigen receptor (CAR).

42. The method of any one of claims 1-39, wherein the autoantigen-binding protein is a multi-chain protein.

43. The method of claim 42, wherein the autoantigen-binding protein is a full-length antibody, a dual-variable immunoglobulin domain, a diabody, a triabody, an antibody-like protein scaffold, a Fab fragment, or a F(ab′)₂ molecule.

44. The method of any one of claims 1-43, wherein the autoimmune disease is type 1 diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barré, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Ménière's Disease, Mixed Connective Tissue Disease, Multiple Sclerosis, Myasthenia Gravis, Neuromyelitis Optica, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, or Wegener's Granulomatosis.

45. The method of any one of claims 1-44, wherein the autoantigen is myelin oligodendrocyte glycoprotein, aquaporin 4, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double-stranded DNA, single-stranded DNA, transferrin, fetuin, factor II, factor VII, fibrin, fibrinogen, C₁, C₁q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

46. The method of claim 44, wherein the autoimmune disease is multiple sclerosis and the autoantigen is myelin oligodendrocyte glycoprotein.

47. The method of claim 44, wherein the autoimmune disease is type 1 diabetes and the autoantigen is insulin, GAD-65, IA-2, or ZnT8.

48. The method of claim 44, wherein the autoimmune disease is rheumatoid arthritis and the autoantigen is collagen II, the Fc portion of immunoglobin, citrullinated peptides, carbamylated peptides, or HSP65.

49. The method of claim 44, wherein the autoimmune disease is myasthenia gravis and the autoantigen is AChR, MuSK, or LRP4.

50. The method of claim 44, wherein the autoimmune disease is lupus and the autoantigen is histone II A.

51. The method of claim 44, wherein the autoimmune disease is hypothyroidism and the autoantigen is a protein expressed in the thyroid gland.

52. The method of claim 44, wherein the autoimmune disease is Graves' disease and the autoantigen is the thyrotrophin receptor.

53. The method of claim 44, wherein the autoimmune disease is pemphigus vulgaris and the autoantigen is double-stranded DNA.

54. The method of claim 44, wherein the autoimmune disease is psoriasis and the autoantigen is double-stranded DNA.

55. The method of claim 44, wherein the autoimmune disease is neuromyelitis optica and the autoantigen is aquaporin 4.

56. The method of any one of claims 1-55, wherein upon administration of the population of pluripotent hematopoietic cells to the patient, the administered cells, or progeny thereof, differentiate into CD4+CD25+ Treg cells.

57. The method of any one of claims 1-56, wherein the patient is a mammal and the cells are mammalian cells.

58. The method of claim 57, wherein the mammal is a human and the cells are human cells.

59. A pharmaceutical composition comprising (i) a population of pluripotent hematopoietic cells comprising a nucleic acid cassette that encodes an autoantigen-binding protein, wherein the nucleic acid cassette is operably linked to one or more lineage-specific transcription regulatory elements that are active in CD4+CD25+ Treg cells, and (ii) one or more pharmaceutically acceptable excipients, carriers, or diluents.

60. The pharmaceutical composition of claim 59, wherein the pluripotent hematopoietic cells are HSCs or HPCs.

61. The pharmaceutical composition of claim 59, wherein the pluripotent hematopoietic cells are embryonic stem cells.

62. The pharmaceutical composition of claim 59, wherein the pluripotent hematopoietic cells are induced pluripotent stem cells.

63. The pharmaceutical composition of claim 59, wherein the pluripotent hematopoietic cells are lymphoid progenitor cells.

64. The pharmaceutical composition of any one of claims 59-63, wherein the pluripotent hematopoietic cells are CD34+ cells.

65. The pharmaceutical composition of any one of claims 59-64, wherein the pluripotent hematopoietic cells are transduced ex vivo with a viral vector comprising the nucleic acid cassette that encodes the autoantigen-binding protein.

66. The pharmaceutical composition of any one of claims 59-64, wherein the pluripotent hematopoietic cells are transfected ex vivo with a polynucleotide comprising the nucleic acid cassette that encodes the autoantigen-binding protein.

67. The pharmaceutical composition of any one of claims 59-64, wherein the pluripotent hematopoietic cells are obtained by delivering to the cells a nuclease that catalyzes a single-strand break or a double-strand break at a target position within the genome of the cell.

68. The pharmaceutical composition of claim 67, wherein the nuclease is delivered to the cells in combination with a gRNA that hybridizes to the target position within the genome of the cell.

69. The pharmaceutical composition of claim 67 or 68, wherein the nuclease is a CRISPR-associated protein.

70. The pharmaceutical composition of claim 69, wherein the CRISPR-associated protein is Cas9 or Cas12a.

71. The pharmaceutical composition of any one of claims 67-70, wherein while the cells are contacted with the nuclease, the cells are additionally contacted with a template polynucleotide comprising the nucleic acid cassette that encodes the autoantigen-binding protein.

72. The pharmaceutical composition of claim 71, wherein the template polynucleotide comprises a 5′ homology arm and a 3′ homology arm having nucleic acid sequences that are sufficiently similar to the nucleic acid sequences located 5′ to the target position and 3′ to the target position, respectively, to promote homologous recombination.

73. The pharmaceutical composition of claim 71 or 72, wherein the nuclease, gRNA, and/or template polynucleotide are delivered to the cells by contacting the cells with a viral vector that encodes the nuclease, gRNA, and/or template polynucleotide.

74. The pharmaceutical composition of any one of claims 59-73, wherein the one or more lineage-specific transcription regulatory elements comprise a Foxp3 promoter.

75. The pharmaceutical composition of claim 74, wherein the Foxp3 promoter has the nucleic acid sequence of SEQ ID NO: 1, SEQ ID NO: 2, SEQ ID NO: 3, or SEQ ID NO: 4, or a nucleic acid sequence that is at least 85% identical thereto.

76. The pharmaceutical composition of claim 74 or 75, wherein the Foxp3 promoter specifically binds transcription factor Nr4a and/or Foxo.

77. The pharmaceutical composition of any one of claims 59-76, wherein the one or more lineage-specific transcription regulatory elements comprise a CNS1 enhancer.

78. The pharmaceutical composition of claim 77, wherein the CNS1 enhancer has the nucleic acid sequence of SEQ ID NO: 5, SEQ ID NO: 6, SEQ ID NO: 7, or SEQ ID NO: 8, or a nucleic acid sequence that is at least 85% identical thereto.

79. The pharmaceutical composition of claim 77 or 78, wherein the CNS1 enhancer specifically binds transcription factor AP-1, NFAT, Smad3, and/or Foxo.

80. The pharmaceutical composition of any one of claims 59-79, wherein the one or more lineage-specific transcription regulatory elements comprise a CNS2 enhancer.

81. The pharmaceutical composition of claim 80, wherein the CNS2 enhancer has the nucleic acid sequence of SEQ ID NO: 9, SEQ ID NO: 10, SEQ ID NO: 11, or SEQ ID NO: 12, or a nucleic acid sequence that is at least 85% identical thereto.

82. The pharmaceutical composition of claim 80 or 81, wherein the CNS2 enhancer specifically binds transcription factor Runx, Foxp3, Ets-1, CREB, Stat5, NFAT, and/or c-Rel.

83. The pharmaceutical composition of any one of claims 59-82, wherein the one or more lineage-specific transcription regulatory elements comprise a CNS3 enhancer.

84. The pharmaceutical composition of claim 83, wherein the CNS3 enhancer has the nucleic acid sequence of SEQ ID NO: 13, SEQ ID NO: 14, SEQ ID NO: 15, or SEQ ID NO: 16, or a nucleic acid sequence that is at least 85% identical thereto.

85. The pharmaceutical composition of claim 83 or 84, wherein the CNS3 enhancer specifically binds transcription factor Foxo and/or c-Rel.

86. The pharmaceutical composition of any one of claims 59-85, wherein the one or more lineage-specific transcription regulatory elements comprise a CNS0 enhancer.

87. The pharmaceutical composition of claim 86, wherein the CNS0 enhancer has the nucleic acid sequence of SEQ ID NO: 17, SEQ ID NO: 18, SEQ ID NO: 19, or SEQ ID NO: 20, or a nucleic acid sequence that is at least 85% identical thereto.

88. The pharmaceutical composition of claim 86 or 87, wherein the CNS0 enhancer specifically binds transcription factor Satb1 and/or Stat5.

89. The pharmaceutical composition of any one of claims 59-88, wherein the autoantigen-binding protein is a single-chain polypeptide.

90. The pharmaceutical composition of any one of claims 59-89, wherein the autoantigen-binding protein is a CAR.

91. The pharmaceutical composition of any one of claims 59-88, wherein the autoantigen-binding protein is a multi-chain protein.

92. The pharmaceutical composition of claim 91, wherein the autoantigen-binding protein is a full-length antibody, a dual-variable immunoglobulin domain, a diabody, a triabody, an antibody-like protein scaffold, a Fab fragment, or a F(ab′)₂ molecule.

93. The pharmaceutical composition of any one of claims 59-92, wherein the autoantigen is myelin oligodendrocyte glycoprotein, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double stranded DNA, single stranded DNA, transferrin, fetuin, factor II, factor VII, fibrin, fibrinogen, C₁, C₁q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

94. A kit comprising the pharmaceutical composition of any one of claims 59-93, wherein the kit further comprises a package insert instructing a user of the kit to administer the pharmaceutical composition to a human patient having an autoimmune disease.

95. The kit of claim 94, wherein the package insert instructs a user of the kit to perform the method of any one of claims 1-58.

96. A nucleic acid cassette encoding an autoantigen-binding protein, wherein the nucleic acid cassette is operably linked to one or more lineage-specific transcription regulatory elements that are active in CD4+CD25+ Treg cells.

97. The nucleic acid cassette of claim 96, wherein the one or more lineage-specific transcription regulatory elements comprise a Foxp3 promoter.

98. The nucleic acid cassette of claim 97, wherein the Foxp3 promoter has the nucleic acid sequence of SEQ ID NO: 1, SEQ ID NO: 2, SEQ ID NO: 3, or SEQ ID NO: 4, or a nucleic acid sequence that is at least 85% identical thereto.

99. The nucleic acid cassette of claim 97 or 98, wherein the Foxp3 promoter specifically binds transcription factor Nr4a and/or Foxo.

100. The nucleic acid cassette of any one of claims 96-99, wherein the one or more lineage-specific transcription regulatory elements comprise a CNS1 enhancer.

101. The nucleic acid cassette of claim 100, wherein the CNS1 enhancer has the nucleic acid sequence of SEQ ID NO: 5, SEQ ID NO: 6, SEQ ID NO: 7, or SEQ ID NO: 8, or a nucleic acid sequence that is at least 85% identical thereto.

102. The nucleic acid cassette of claim 100 or 101, wherein the CNS1 enhancer specifically binds transcription factor AP-1, NFAT, Smad3, and/or Foxo.

103. The nucleic acid cassette of any one of claims 96-102, wherein the one or more lineage-specific transcription regulatory elements comprise a CNS2 enhancer.

104. The nucleic acid cassette of claim 103, wherein the CNS2 enhancer has the nucleic acid sequence of SEQ ID NO: 9, SEQ ID NO: 10, SEQ ID NO: 11, or SEQ ID NO: 12, or a nucleic acid sequence that is at least 85% identical thereto.

105. The nucleic acid cassette of claim 103 or 104, wherein the CNS2 enhancer specifically binds transcription factor Runx, Foxp3, Ets-1, CREB, Stat5, NFAT, and/or c-Rel.

106. The nucleic acid cassette of any one of claims 96-105, wherein the one or more lineage-specific transcription regulatory elements comprise a CNS3 enhancer.

107. The nucleic acid cassette of claim 106, wherein the CNS3 enhancer has the nucleic acid sequence of SEQ ID NO: 13, SEQ ID NO: 14, SEQ ID NO: 15, or SEQ ID NO: 16, or a nucleic acid sequence that is at least 85% identical thereto.

108. The nucleic acid cassette of claim 106 or 107, wherein the CNS3 enhancer specifically binds transcription factor Foxo and/or c-Rel.

109. The nucleic acid cassette of any one of claims 96-108, wherein the one or more lineage-specific transcription regulatory elements comprise a CNS0 enhancer.

110. The nucleic acid cassette of claim 109, wherein the CNS0 enhancer has the nucleic acid sequence of SEQ ID NO: 17, SEQ ID NO: 18, SEQ ID NO: 19, or SEQ ID NO: 20, or a nucleic acid sequence that is at least 85% identical thereto.

111. The nucleic acid cassette of claim 109 or 110, wherein the CNS0 enhancer specifically binds transcription factor Satb1 and/or Stat5.

112. The nucleic acid cassette of any one of claims 96-111, wherein the autoantigen-binding protein is a single-chain polypeptide.

113. The nucleic acid cassette of any one of claims 96-112, wherein the autoantigen-binding protein is a CAR.

114. The nucleic acid cassette of any one of claims 96-111, wherein the autoantigen-binding protein is a multi-chain protein.

115. The nucleic acid cassette of claim 114, wherein the autoantigen-binding protein is a full-length antibody, a dual-variable immunoglobulin domain, a diabody, a triabody, an antibody-like protein scaffold, a Fab fragment, or a F(ab′)₂ molecule.

116. The nucleic acid cassette of any one of claims 96-115, wherein the autoantigen is myelin oligodendrocyte glycoprotein, actin, tubulin, myosin, tropomyosin, vimentin, fibronectin, collagen I, collagen II, collagen III, collagen IV, collagen V, heparin, laminin, collagenase, cardiolipin, glucocerebroside, phosphatidylethanolamine, cholesterol, enolase, aldolase, acid phosphatase, annexin 33 kDa, annexin 67 kDa, cytochrome P450C, catalase, peroxidase, tyrosinase, ribonuclease, histone II A, double stranded DNA, single stranded DNA, transferrin, fetuin, factor II, factor VII, fibrin, fibrinogen, C₁, C₁q, interleukin 2, interleukin 10, interleukin 4, interferon-γ, TNFαR, HSP60, HSP65, GAD, insulin, IA-2, ZnT8, MBP, AchR, myoglobulin, thyroglobulin, hemoglobin A, spectrin, TB PPD, LPS, MuSK, LRP4, the Fc portion of immunoglobin, citrullinated peptides, carbamylated peptides, the thyrotrophin receptor, or a protein expressed in the thyroid gland.

117. A viral vector comprising the nucleic acid cassette of any one of claims 96-116.
